SEQUENCE LISTING (3 sequences)

SEQ ID NO: 1 - protein, 396 aa, homo sapiens
SMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWIS

SEQ ID NO: 2 - protein, 1226 aa, homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 3 - protein, 29 aa, artificial sequence
  synthetic peptide
ALFDKSCSDRAMESTESRHYPVFENPKQG

(19) United States
(12) Patent Application Publication
(10) Pub. No.: US 2024/0108734 A1
(43) Pub. Date: Apr. 4, 2024

(54) METHODS FOR TREATING AND AMELIORATING CANCER

(71) Applicant: The Regents of the University of California, Oakland, CA (US)

(72) Inventors: Catriona JAMIESON, San Diego, CA (US); Wenxue MA, San Diego, CA (US)

(21) Appl. No.: 18/273,716

(22) PCT Filed: Jan. 21, 2022
(86) PCT No.: PCT/US2022/013398

§ 371 Date: Jul. 21, 2023

Related U.S. Application Data
(60) Provisional application No. 63/140,725, filed on Jan. 22, 2021.

(51) Int. Cl. A61K 47/54; A61K 9/127; A61K 31/365; A61K 31/506; A61K 31/519; A61K 31/5383; A61K 31/635; A61K 31/7064; A61K 35/28; A61K 38/50; A61K 45/06; A61M 15/08; A61P 31/14; A61P 31/16; A61P 35/02; C12N 15/113; C12N 15/86

(52) U.S. Cl. CPC A61K 47/549; A61K 9/127; A61K 31/365; A61K 31/506; A61K 31/519; A61K 31/5383; A61K 31/635; A61K 31/7064; A61K 35/28; A61K 38/50; A61K 45/06; A61M 15/08; A61P 31/14; A61P 31/16; A61P 35/02; C12N 15/1137; C12N 15/86; C12Y 305/04; C12N 2310/14; C12N 2310/531; C12N 2740/15043

(57) ABSTRACT

In alternative embodiments, provided are methods for treating and ameliorating a cancer such as a leukemia such as acute myeloid leukemia (AML) comprising administration to an individual in need thereof a pharmaceutical composition comprising imetelstat, or imetelstat and second drug such as dasatinib, or ruxolitinib, fedratinib, 8-aza-adenosine, raltegravir and/or dolutegravir or any combination thereof. In alternative embodiments, provided are methods for the in vivo inhibition of myeloproliferative neoplasm (MPN) or AML stem cell propagation comprising administration to an individual in need thereof a pharmaceutical composition comprising imetelstat, or imetelstat and second drug. In alternative embodiments, provided are methods for the in vivo inhibition pre-leukemia stem cell (pre-LSC) transformation into leukemia stem cells (LSCs) comprising administration to an individual in need thereof a pharmaceutical composition comprising imetelstat, or imetelstat and second drug such as dastinib, or ruxolitinib, fedratinib, 8-aza-adenosine, raltegravir and/or dolutegravir or any combination thereof.

### RELATED APPLICATIONS

This Patent Convention Treaty (PCT) International Application claims the benefit of priority under 35 U.S.C. § 119(e) of U.S. Provisional Application No. 63/140,725, Jan. 22, 2021. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under RO1 114468 and RO1 CA 205944 awarded by the National Institutes of Health (NIH). The government has certain rights in the invention.

### TECHNICAL FIELD

This invention generally relates to medicine. In alternative embodiments, provided are methods for treating and ameliorating a cancer such as a leukemia such as acute myeloid leukemia (AML) comprising administration to an individual in need thereof a pharmaceutical composition comprising imetelstat, or imetelstat and second drug such as an ATP-competitive protein tyrosine kinase inhibitor such as dasatinib, or comprising ruxolitinib, fedratinib, 8-aza-adenosine, raltegravir and/or dolutegravir or any combination thereof. In alternative embodiments, provided are methods for the in vivo inhibition of a cancer or neoplasm, for example, myeloproliferative neoplasm (MPN) or AML stem cell propagation comprising administration to an individual in need thereof a pharmaceutical composition comprising imetelstat, or imetelstat and second drug such as dastinib, or ruxolitinib, fedratinib, 8-aza-adenosine, raltegravir and/or dolutegravir or any combination thereof. In alternative embodiments, provided are methods for the in vivo inhibition pre-leukemia stem cell (pre-LSC) transformation into leukemia stem cells (LSCs) comprising administration to an individual in need thereof a pharmaceutical composition comprising imetelstat, or imetelstat and second drug such as dastinib, or ruxolitinib, fedratinib, 8-aza-adenosine, raltegravir and/or dolutegravir or any combination thereof.

### BACKGROUND

Clonal stem cell derived myeloproliferative neoplasms (MPNs) have varying propensities to progress to acute myeloid leukemia (AML). Previous studies show that beta-catenin induces LSC generation and that beta-catenin regulates TERT expression.

By protecting chromosome ends from degradation, human telomerase reverse transcriptase (hTERT) forms part of a complex that regulates genomic integrity and hematopoietic stem cell (HSC) longevity. As repetitive nucleotide sequences consisting of 8 to 15 kb random repeats (5′-TTAGGG-3′) in the beginning that reduce to 50 to 200 nucleotides after each replication cycle, telomere regulation is critical for preventing stem cell senescence and cancer progression.

Previous studies in embryonic stem cells show that beta-catenin cooperates with Klf4 to binding to the Tert promoter.

Human myeloproliferative neoplasm stem and progenitor cell (MPN-SPC) transformation into long-lived, self-renewing leukemia stem cells (LSCs) is fueled, at least in part, by ADAR1 (adenosine deaminase acting on RNA-1) and β-catenin, which transcriptionally activates telomerase reverse transcriptase (TERT).

### SUMMARY

In alternative embodiments, provided methods for:
treating and ameliorating a cancer or a cancer stem cell, wherein optionally the cancer is a leukemia, optionally acute myeloid leukemia (AML) or a myeloproliferative neoplasm (MPN);
in vivo inhibition of myeloproliferative neoplasm (MPN) or AML stem cell propagation; or
the in vivo inhibition pre-leukemia stem cell (pre-LSC) transformation into leukemia stem cells (LSCs),
comprising:
administration to an individual in need thereof a formulation, a pharmaceutical composition or therapeutic combination of drugs, comprising:
(a) imetelstat or imetelstat sodium, or imetelstat or imetelstat sodium and at least one second drug, optionally the second drug comprises an ATP-competitive protein tyrosine kinase inhibitor;
(b) a JAK2 (Janus kinase 2) inhibitor, optionally ruxolitinib (or JAKAFI™, or OPZELURA™), or ruxolitinib and at least one second drug,
(c) a JAK2 (Janus kinase 2) inhibitor, optionally fedratinib (or INREBIC™) or fedratinib and at least one second drug, wherein optionally the fedratinib is dosage at 60 mg/kg twice daily orally, optionally for one to two or more weeks,
(d) 8-aza-adenosine, or 8-aza-adenosine and at least one second drug,
(e) an integrase inhibitor, or raltegravir (or ISENTRESS™), or raltegravir and at least one second drug,
(f) an integrase inhibitor, or dolutegravir (or TIVICAY™), or dolutegravir and at least one second drug,
(g) an ADAR1 (adenosine deaminase acting on RNA-1) inhibiting agent, wherein optionally the ADAR1 inhibiting (inhibitory) agent comprises an ADAR1 inhibiting nucleic acid, optionally an antisense ADAR1 or a small inhibitory ADAR1 RNA,
and optionally the ADAR1 inhibiting (inhibitory) nucleic acid is contained in and expressed by a vector, optionally a lentiviral vector, optionally a lentiviral shRNA ADAR1 knockdown vector, a lentiviral ADAR1 inhibitory mutant vector, a lentiviral ADAR1 Z alpha domain deleted vector, or a lentiviral JAK2 overexpression vector,
and optionally the ADAR1 inhibiting (inhibitory) comprises an interferon inhibitory compound;
(h) a compound having the formula,

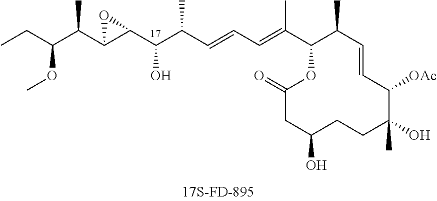 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

or an enantiomer, stereoisomer, deuterated version, or salt thereof,
(i) any combination thereof, and optionally the therapeutic combination of drugs comprise (a) and (b), (a) and (c), (a) and (d), (a) and (e), (a) and (f), (a) and (g), (a) and (h), (b) and (c), (b) and (d), (b) and (e), (b) and (f), (b) and (g), (b) and (h), (c) and (d), (c) and (e), (c) and (f) and (c) and (g), (c) and (h), (d) and (e), (d) and (f), (d) and (g), (d) and (h), (e) and (f), (e) and (g), (e) and (h), (f) and (g), (f) and (h), and/or (g) and (h).

In alternative embodiments of methods as provided herein:
the at least one second drug comprises an ATP-competitive protein tyrosine kinase inhibitor, wherein optionally the ATP-competitive protein tyrosine kinase inhibitor comprises dasatinib (or SPRYCEL™ or DASANIX™);
the at least one second drug comprises a chemotherapeutic agent, wherein optionally the chemotherapeutic agent comprises one, two, three or more of: afatinib (or GILOTRIF™), afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, fedratinib (or INREBIC™), floxuridine, fludarabine, fluorouracil, gefitinib, gemcitabine, hydroxyurea, ibrutinib, icotinib, idarubicin, ifosfamide, imatinib, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib (or OPZELURA™), seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (or ZELBORAE™), vincristine, vinblastine, vinorelbine, and/or vindesine;
the at least one second drug comprises a hypomethylating agent (HMA), wherein optionally the HMA comprises azacitidine or decitabine;
the at least one second drug comprises a second telomerase inhibitor, wherein optionally the telomerase inhibitor comprises at least one, two or three of: zidovudine, stavudine, tenofovir, didanosine, abacavir, TMPI, telomestatin, RHPS4, BRACO-19, TMPyP4, tertomotide, ASTVAC-1, GX-301, UCPVax, UV-1, Vx-001, Vx-006, INO-1400, INVAC-1, ASTVAC-2, Telin(ab 4,4-dichloro-1-(2,4-dichlorophenyl)-3-methyl-5-pyrazolone), Vbx-011, Vbx-021, Vbx-026INO-5401, KML-001, TK-005, Ribovax, Vbx-016, ZI-HX, ZI-H04, and ZIH-03;
the formulation, pharmaceutical composition or therapeutic combination of drugs or an active agent or drug contained therein is or are formulated or contained in: a liquid formulation (optionally sterile saline or water), a spray, a powder, an aerosol, a mist, or any formulation for inhalation, a pill, a capsule, a tablet, or a geltab, or equivalents; or, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; or, a drug delivery device or package, blister pack, clamshell or tray comprising a plurality of compartments spatially arranged on the drug delivery device or package, blister pack, clamshell or tray to follow a dosage administration regimen;
an active agent or drug in the formulation, pharmaceutical composition or therapeutic combination of drugs is dosages at between about 10 to 500 mg/day, or between about 500 to 1 gram a day, or at a dosage of between about 100 to 600 mg per day or per dosage, or at about 100, 200, 300, 400, 500 or 600 mg per day or per dosage, and optionally a unit dosage is administered to an individual in need thereof once a day (QD), or twice a day (BID), or three times a day (TID), or more; and/or
an active agent or drug in the formulation, pharmaceutical composition or therapeutic combination of drugs is administered as or formulated with or formulated as an) inhaled or aerosol formulation such as a powder or a mist or aerosol, and/or is formulated with or formulated as an oral, intramuscular (IM), subcutaneous (SC), intrathecal or intravenous (IV) formulation, wherein optionally both the inhaled (or aerosol) and the oral, IV, SC, intrathecal and/or IM formulations are administered simultaneously or sequentially.

In alternative embodiments, provided are uses of a formulation, pharmaceutical composition or therapeutic combination of drugs, comprising:
(a) imetelstat or imetelstat sodium, or imetelstat or imetelstat sodium and at least one second drug, and optionally the second drug comprises an ATP-competitive protein tyrosine kinase inhibitor;
(b) a JAK2 (Janus kinase 2) inhibitor, optionally ruxolitinib (or JAKAFI™, or OPZELURA™), or ruxolitinib and at least one second drug,
(c) a JAK2 (Janus kinase 2) inhibitor, optionally fedratinib (or INREBIC™) or fedratinib and at least one second drug,
(d) 8-aza-adenosine, or 8-aza-adenosine and at least one second drug,
(e) an integrase inhibitor, or raltegravir (or ISENTRESS™), or raltegravir and at least one second drug,
(f) an integrase inhibitor, or dolutegravir (or TIVICAY™), or dolutegravir and at least one second drug,
(g) an ADAR1 (adenosine deaminase acting on RNA-1) inhibiting agent, wherein optionally the ADAR1 inhibiting (inhibitory) agent comprises an ADAR1 inhibiting nucleic acid, optionally an antisense ADAR1 or a small inhibitory ADAR1 RNA,
and optionally the ADAR1 inhibiting (inhibitory) nucleic acid is contained in and expressed by a vector, optionally a lentiviral vector, optionally a lentiviral shRNA ADAR1 knockdown vector, a lentiviral ADAR1 inhibitory mutant vector, a lentiviral ADAR1 Z alpha domain deleted vector, or a lentiviral JAK2 overexpression vector,
and optionally the ADAR1 inhibiting (inhibitory) comprises an interferon inhibitory compound;
(h) any combination thereof, and optionally the therapeutic combination of drugs comprise (a) and (b), (a) and (c), (a) and (d), (a) and (e), (a) and (f), (a) and (g), (b) and (c), (b) and (d), (b) and (e), (b) and (f), (b) and (g), (c) and (d), (c) and (e), (c) and (f) and (c) and (g), (d) and (e), (d) and (f), and (d) and (g), (e) and (f), (e) and (g), or (f) and (g) for:
   treating and ameliorating a cancer, wherein optionally the cancer is a leukemia, optionally acute myeloid leukemia (AML) or a myeloproliferative neoplasm (MPN);
   in vivo inhibition of myeloproliferative neoplasm (MPN) or AML stem cell propagation; or
   the in vivo inhibition pre-leukemia stem cell (pre-LSC) transformation into leukemia stem cells (LSCs).

In alternative embodiments, provided are: a formulation, pharmaceutical composition or therapeutic combination of drugs for use in:
treating and ameliorating a cancer, wherein optionally the cancer is a leukemia, optionally acute myeloid leukemia (AML) or a myeloproliferative neoplasm (MPN);
in vivo inhibition of myeloproliferative neoplasm (MPN) or AML stem cell propagation; or
the in vivo inhibition pre-leukemia stem cell (pre-LSC) transformation into leukemia stem cells (LSCs), the pharmaceutical composition or therapeutic combination of drugs comprising:
(a) imetelstat or imetelstat sodium, or imetelstat or imetelstat sodium and at least one second drug, and optionally the second drug comprises an ATP-competitive protein tyrosine kinase inhibitor;
(b) a JAK2 (Janus kinase 2) inhibitor, optionally ruxolitinib (or JAKAFI™, or OPZELURA™), or ruxolitinib and at least one second drug,
(c) a JAK2 (Janus kinase 2) inhibitor, optionally fedratinib (or INREBIC™) or fedratinib and at least one second drug,
(d) 8-aza-adenosine, or 8-aza-adenosine and at least one second drug, (e) an integrase inhibitor, or raltegravir (or ISENTRESS™), or raltegravir and at least one second drug,
(f) an integrase inhibitor, or dolutegravir (or TIVICAY™), or dolutegravir and at least one second drug,
(g) an ADAR1 (adenosine deaminase acting on RNA-1) inhibiting agent, wherein optionally the ADAR1 inhibiting (inhibitory) agent comprises an ADAR1 inhibiting nucleic acid, optionally an antisense ADAR1 or a small inhibitory ADAR1 RNA,
and optionally the ADAR1 inhibiting (inhibitory) nucleic acid is contained in and expressed by a vector, optionally a lentiviral vector, optionally a lentiviral shRNA ADAR1 knockdown vector, a lentiviral ADAR1 inhibitory mutant vector, a lentiviral ADAR1 Z alpha domain deleted vector, or a lentiviral JAK2 overexpression vector,
and optionally the ADAR1 inhibiting (inhibitory) comprises an interferon inhibitory compound;
(h) any combination thereof, and optionally the therapeutic combination of drugs comprise (a) and (b), (a) and (c), (a) and (d), (a) and (e), (a) and (f), (a) and (g), (b) and (c), (b) and (d), (b) and (e), (b) and (f), (b) and (g), (c) and (d), (c) and (e), (c) and (f) and (c) and (g), (d) and (e), (d) and (f), and (d) and (g), (e) and (f), (e) and (g), or (f) and (g).

In alternative embodiments, provided are methods for inhibiting replication of a virus, or for treating or ameliorating, or lessoning the symptoms of, or slowing the progress of, a viral infection in an individual in need thereof, wherein optionally the virus is flu (influenza) virus, a DNA or an RNA virus, a coronavirus, optionally a SARs-CoV-2 or COVID-19 virus or variant thereof, or a retrovirus, comprising:
administering to the individual in need thereof:
a vector or a recombinant virus, optionally a recombinant lentivirus or adenovirus or adeno-associated virus (AAV) vector, expressing or overexpressing (or capable of expressing or overexpressing) ADAR1 or an ADAR1 catalytic domain, wherein optionally the vector or recombinant virus are administered intravenously (IV), or
a transduced stem cell comprising (or substantially comprising) cord blood CD34+ cells or mesenchymal stromal cells, wherein the cord blood CD34+ cells or mesenchymal stromal cells have contained therein a vector or a recombinant virus, optionally a recombinant lentivirus, expressing or overexpressing (or capable of expressing or overexpressing) ADAR1, wherein optionally the cord blood CD34+ cells or mesenchymal stromal cells are administered intravenously (IV).

In alternative embodiments, provided are methods for inhibiting replication of a virus, or for treating or ameliorating, or lessoning the symptoms of, or slowing the progress of, a viral infection in an individual in need thereof, wherein optionally the virus is flu (influenza) virus, a DNA or an RNA virus, a coronavirus, optionally a SARs-CoV-2 or COVID-19 virus or variant thereof, or a retrovirus, comprising: administering to the individual in need thereof: a ADAR1 full length protein, or an ADAR1 catalytic domain, or a ADAR1 Z alpha domain deleted-protein, contained in a liposome or equivalent lipid vesicle by intravenous administration or inhalation.

In alternative embodiments of methods as provided herein, a vector or recombinant virus, or liposome or equivalent lipid vesicle, or formulation, pharmaceutical composition or therapeutic combination of drugs, is or are administered to an individual in need thereof: using a drug delivery device, optionally by inhalation, wherein the drug delivery device optionally comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device, or, intravenously (IV) or intramuscularly (IM).

In alternative embodiments, provided are uses of a vector or a recombinant virus, optionally a recombinant lentivirus or adenovirus or adeno-associated virus (AAV) vector, expressing or overexpressing (or capable of expressing or overexpressing) ADAR1 or an ADAR1 catalytic domain, wherein optionally the vector or recombinant virus are administered intravenously (IV), for inhibiting replication of a virus, or for treating or ameliorating, or lessoning the symptoms of, or slowing the progress of, a viral infection in an individual in need thereof, wherein optionally the virus is flu (influenza) virus, a DNA or an RNA virus, a coronavirus, optionally a SARs-CoV-2 or COVID-19 virus or variant thereof, or a retrovirus, wherein the use comprises: administering to the individual in need thereof: a transduced stem cell comprising (or substantially comprising) cord blood CD34+ cells or mesenchymal stromal cells, wherein the cord blood CD34+ cells or mesenchymal stromal cells have contained therein a vector or a recombinant virus, optionally a recombinant lentivirus, expressing or overexpressing (or capable of expressing or overexpressing) ADAR1, wherein optionally the cord blood CD34+ cells or mesenchymal stromal cells are administered intravenously (IV).

In alternative embodiments, provided are uses of a ADAR1 full length protein, or an ADAR1 catalytic domain, or a ADAR1 Z alpha domain deleted-protein, contained in a liposome or equivalent lipid vesicle by intravenous administration or inhalation for inhibiting replication of a virus, or for treating or ameliorating, or lessoning the symptoms of, or slowing the progress of, a viral infection in an individual in need thereof, wherein optionally the virus is flu (influenza) virus, a DNA or an RNA virus, a coronavirus, optionally a SARs-CoV-2 or COVID-19 virus or variant thereof, or a retrovirus.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

Figures are described in detail herein.

FIG. 1A-D illustrate the quantification of telomerase activation in MPN Stem and progenitor dells (MPN-SPC):

FIG. 1A graphically illustrates the age dependency of telomere content in CD34⁺ stem cells and bulk saliva, as quantified by whole genome sequencing (WGS) analysis;

FIG. 1B graphically illustrates a comparative WGS analysis between age adjusted telomere lengths in males and females in both CD34⁺ stem cells and bulk saliva, and the boxplots depict the distribution of age adjusted telomere lengths for males and females;

FIG. 1C graphically illustrates a WGS analysis comparison of age adjusted telomere lengths during MPN progression in CD34⁺ stem cells and bulk saliva, and the boxplots depict the distribution of age adjusted telomere lengths for different stages of MPN;

FIG. 1D left image graphically illustrates a boxplot depicting TERT expression in stem cells and progenitors that were FACS-purified from normal young (YBM) and aged (ABM) bone marrow and subjected to RNA-seq analysis, and

FIG. 1D right image graphically illustrates TERT gene expression levels by RNA-seq for ABM and YBM (n=24), ET (n=2), PV (n=6), MF (n=29) and sAML (n=12) progenitors,

as discussed in detail in Example 1, below.

FIG. 2A-G illustrate data showing that ADAR1p150, beta-catenin and hTERT upregulation characterize MPN-SPC:

FIG. 2A left image graphically illustrates the correlation of normalized log transformed counts per million (Log cpm) RNA-seq expression data for TERT and ADAR p110 isoform in FACS purified stem cells from aged bone marrow (ABM, red), young bone marrow (YBM, yellow), essential thrombocythemia (ET, green), polycythemia vera (PV, teal), acute myeloid leukemia (AML, pink) and chronic myeloid leukemia (CML, purple);

FIG. 2A right image graphically illustrates TERT and ADAR1 p150 expression levels (Log cpm) in stem cells that were FACS purified from aged bone marrow (ABM, red), young bone marrow (YBM, green), intermediate MF (int-MF, blue) and high risk MF (HR MF, purple);

FIG. 2B graphically illustrates correlation of normalized log transformed counts per million (Log cpm) RNA-seq expression data for TERT and ADAR p110 isoform (left image) or TERT and ADARp150 isoform (right image) in progenitors that were FACS-purified from aged bone marrow (ABM, red), young bone marrow (YBM, green), intermediate MF (int-MF, blue) and high risk MF (HR MF, purple);

FIG. 2C illustrate an image of a confocal fluorescence photomicrograph depicting DAPI (blue, far left), TERT (green, middle left), ADAR1 (red, middle right) and merged images (right) of TERT and ADAR1 localization (yellow);

FIG. 2D illustrate an image of a hierarchical clustering gene expression analysis of RNA-seq data derived from CD34⁺ cord blood cells that were lentivirally transduced with ADAR1 wild-type (ADAR1 WT, orange) or a deaminase defective mutant (ADAR1 E912A Mutant, green);

FIG. 2E graphically illustrates TCF/LEF reporter activity aas measured by firefly luciferase and normalized to cell viability in K562 cells lentivirally transduced with pCDH backbone, ADAR1^{WT}, shControl or shADAR1;

FIG. 2F graphically illustrates self-renewal capacity as measured by colony replating assays in primary myelofibrosis CD34⁺ cells lentivirally transduced with either pCDH backbone (grey bar) or ADAR1^{WT} (red bar); and

FIG. 2G upper image graphically illustrates a FACS plot, or FACS analysis, of mean fluorescence intensity (MFI) of active (non-phosphorylated) beta-catenin levels in BC CML leukemia (K562) cells transduced with lentiviral-GFP vectors expressing ADAR1 (ADAR1^{WT}; red circles or pCDH backbone (blue circles); and,

FIG. 2G lower image graphically illustrates a FACS plot, or FACS analysis, of beta-catenin MFI in KG1a CD34⁺ leukemia cells transduced with shScramble control compared with shADAR1 lentiviral vectors, as discussed in detail in Example 1, below.

FIG. 3A-E illustrate data showing that telomerase inhibition prevents MPN-SPC and LSC maintenance in vitro:

FIG. 3A illustrates a schematic of SL/M2 stromal co-cultures established to quantify in vitro MPN-SPC and LSC survival and self-renewal;

FIG. 3B graphically illustrates imetelstat as used at concentrations ranging from 1 to 10 μM and compared with mismatch controls in aged normal bone marrow (aNBM, n=4, blue) and the BC CML (n=5, red) stromal co-cultures followed by clonogenic survival (upper panel) and self-renewal (lower panel) assays;

FIG. 3C graphically illustrates imetelstat as used at concentrations ranging from 1 to 10 μM alone or in combination with dasatinib (111M) to treat aged normal bone marrow (a-NBM, n=4, black) compared with BC CML (purple, n=5) in stromal co-cultures followed by clonogenic survival (upper panel) and self-renewal (lower panel) assays;

FIG. 3D graphically illustrates imetelstat aas used at concentrations ranging from 1 to 10 μM to treat aged normal bone marrow (aNBM, n=4, dark blue) compared with myelofibrosis (MF, light blue, n=9) in stromal co-cultures followed by clonogenic survival (upper panel) and self-renewal (lower panel) assays;

FIG. 3E graphically illustrates imetelstat aas used at concentrations ranging from 1 to 10 μM alone or in combination with pacritinib (5 nM) to treat aged normal bone marrow (a-NBM, n=4, blue) compared with MF (green, n=9) in stromal co-cultures followed by clonogenic survival (upper panel) and self-renewal (lower panel) assays, as discussed in detail in Example 1, below.

FIG. 4A-I illustrate data showing that telomerase inhibition prevents MPN-SPC and LSC maintenance in vivo:

FIG. 4A illustrates a schematic of humanized BC CML LSC and MF SPC mouse models;

FIG. 4B graphically illustrates bone marrow from each treatment group as collected for histological examination and FACS analysis: Left images are photomicrogaphs depict reticulin staining, DAPI staining, human CD45⁺ expression and merged DAPI and CD45 images in bone marrow collected from no transplant, vehicle, and mismatch controls as well as from imetelstat treated myelofibrosis (MF) mouse models; and right images illustrate FACS analysis plots depicting forward scatter (FSC, y-axis) and human CD45 engraftment (x-axis) in no transplant, vehicle, and mismatch controls as well as imetelstat treated MF mouse models;

FIG. 4C graphically illustrates FACS analysis of percentage of human CD45⁺ cell engraftment (percent) in MF mouse BM after treatment with vehicle (red), mismatch (blue) or imetelstat (green);

FIG. 4D graphically illustrates FACS analysis of percentage of human CD34⁺CD38⁺Lin⁻ progenitor engraftment in mouse bone marrow following MF patient (MF318) sample transplantation and treatment with vehicle (red), mismatch (blue) or imetelstat (green);

FIG. 4E graphically illustrates FACS analysis of percent human CD34⁺ cell engraftment in mice transplanted with pCDH lentiviral backbone (white circles; n=5) or ADAR1 overexpression (ADAR1-OE; red circles; n=6) vector transduced MF progenitor;

FIG. 4F graphically illustrates FACS analysis of percentage of live human CD45⁺ cells in BC CML BM engrafted mice following treatment with vehicle (red), mismatch (blue) or imetelstat (green);

FIG. 4G graphically illustrates FACS analysis of percentage of live human CD34⁺CD38⁺Lin⁻ progenitor cells in BC CML engrafted mice following treatment with vehicle (red), mismatch (blue) or imetelstat (green);

FIG. 4H graphically illustrates self-renewal capacity of human progenitor cells was evaluated in serially transplanted mouse models, and shows a FACS analysis of percentage of live human CD34⁺CD38⁺Lin⁻ progenitor cells following serial engraftment of cells derived from mice treated with vehicle (red), mismatch (blue) or imetelstat (green); and

FIG. 4I graphically illustrates a Kaplan-Meier survival curves of mice transplanted with CD34⁺ cells selected from imetelstat treated mouse BM (green) compared with mismatch controls (blue),

as discussed in detail in Example 1, below.

FIG. 5 illustrates data showing that telomerase inhibition prevents ADAR1-activated LSC propagation:

FIG. 5A graphically illustrates a boxplot depicting AIMP2 Log 2 (cpm) expression by RNA-seq analysis in stem cells (left panel) and progenitors (right panel) from aged bone marrow (ABM), young bone marrow (YBM), PV, ET, MF, CML, sAML and de novo AML (dnAML);

FIG. 5B graphically illustrates a boxplot depicting TRF2IP Log 2 (cpm) expression by RNA-seq analysis in stem cells (left panel) and progenitors (right panel) from aged bone marrow (ABM), young bone marrow (YBM), PV, ET, MF, CML, sAML and de novo AML (dnAML);

FIG. 5C graphically illustrates a boxplot depicting TERT Log 2 (cpm) expression by RNA-seq analysis in human CD45⁺ cells engrafted in vehicle (red), mismatch control (blue) and imetelstat (green) treated BC CML mice;

FIG. 5D graphically illustrates telomerase activity in BC CML engrafted human CD45⁺ cells, as measured by TRAP assay, in vehicle (red), mismatch control (blue) and imetelstat (green) treated BC CML mice;

FIG. 5E graphically illustrates β-catenin activity in human progenitor cells in BC CML mouse BM was measured by flow cytometry in vehicle (red), mismatch control (blue) and imetelstat (green) treated mice;

FIG. 5F graphically illustrates a boxplot depicting Log 2 (cpm) expression of ABL1 by RNA-seq analysis of human CD45⁺ cells isolated from vehicle (red), mismatch control (blue) and imetelstat (green) treated BC CML mouse cells

FIG. 5G graphically illustrates a boxplot depicting Log 2 (cpm) expression of ADAR1p150 by RNA-seq of human CD45⁺ cells isolated from vehicle (red), mismatch control (blue) and imetelstat (green) treated BC CML mouse cells; and

FIG. 5H graphically illustrates a boxplot depicting detectable RNA edits per million aligned reads in human CD45⁺ cells derived from vehicle (red), mismatch control (blue) and imetelstat (green) treated BC CML mouse models, as discussed in detail in Example 1, below.

FIG. 6 schematically illustrates an exemplary S1 RNA sequencing schema, as discussed in detail in Example 1, below.

FIG. 7A-D illustrates data showing the impact of imetelstat on ADAR1 and P-catenin gene expression in BC CML and MF progenitors:

FIG. 7A graphically illustrates ADAR1 expression in BC CML cells using both control and imetelstat;

FIG. 7B graphically illustrates ADAR1 expression in MF cells using both control and imetelstat;

FIG. 7C graphically illustrates beta-catenin expression in BC CML cells using both control and imetelstat;

FIG. 7D graphically illustrates beta-catenin expression in BC CML cells using both control and imetelstat,

as discussed in detail in Example 1, below.

FIG. 8A-L illustrate data showing that imetelstat is well tolerated, decreases splenomegaly in BC CML and inhibits MPN-SPC in xenograft models:

FIG. 8A graphically illustrates data showing the change in percent body weight in mice transplanted with cord blood (CB) treated with vehicle (black), mismatch control (blue) or imetelstat (red);

FIG. 8B graphically illustrates data showing the change in percent body weight in mice transplanted with BC CML treated with vehicle (black), mismatch control (blue) or imetelstat (red);

FIG. 8C graphically illustrates data showing the change in percent body weight in mice transplanted with myelofibrosis (MF) progenitors treated with vehicle (black), mismatch control (blue) or imetelstat (red);

FIG. 8D illustrates photographs of representative mouse spleens from each treatment group (No transplant control, vehicle control, mismatch control, and imetelstat treatment);

FIG. 8E graphically illustrates data showing comparative spleen weight (mg) in the imetelstat treatment group (green) compared with mismatch control (blue, ***p<0.001, ANOVA), and vehicle control (red, ***p<0.001, ANOVA), no transplant control is also shown in black;

FIG. 8F-I graphically illustrate a FACS analysis of imetelstat (white) compared with mismatch (blue) and vehicle control (red) treatment effects on percentage of progenitor cell engraftment in humanized MF (MF318) mouse BM (*p<0.05, ANOVA) (FIG. 8F); humanized MF (MF318) mouse spleen (**p<0.01, ANOVA) (FIG. 8G); percentage stem cell engraftment in humanized MF (MF318) mouse BM (FIG. 8H); percentage of stem cell engraftment in humanized MF (MF318) spleen, **p<0.01, ANOVA (FIG. 8I).

FIG. 8J graphically illustrates colony formation (survival, %) of primary high-risk MF CD34⁺ selected cells transduced with ADAR1 or pCDH reporter for 48-hours prior to Imetelstat (5 μm) or mismatch control (5 μM) treatment on stromal co-culture, all values are normalized to mismatch control, graph shows mean+/−SD.

FIG. 8K graphically illustrates primary high-risk MF and an accelerated phase (AP) CD34⁺ cells transduced with ADAR1 or pCDH reporter treated with Imetelstat (5 μm) or mismatch control (5 μM) treatment on stromal co-culture, secondary colony formation (self-renewal, %), all values are normalized to mismatch control, and

FIG. 8L graphically illustrates ADAR1 reporter activity measured by nanoluciferase and normalized to cell viability. Primary high-risk MF CD34⁺ selected cells were lentivirally transduced with ADAR1 reporter or pCDH backbone for 48-hours prior to M/M or imetelstat treatment on stromal co-culture, reporter activity was measured 72-hours post-treatment.

FIG. 9 illustrates data showing that imetelstat treatment does not reduce human CD45⁺ cells or P-catenin in normal HSC engrafted mouse models:

FIG. 9A-B graphically illustrate data showing that normal HSC mouse xenograft models were established with normal cord blood CD34⁺ cells by intrahepatic injection into neonatal Rag2^{-/-} c^{-/-} mice; FACS analysis of mice treated with either vehicle (0.9% NS), mismatch control (30 mg/kg) or imetelstat (30 mg/kg) followed by quantification of human CD45⁺ cells in BM (FIG. 9A) and in spleen (FIG. 9B);

FIG. 9C-D graphically illustrate B-catenin activity in human CD45⁺ cells (FIG. 9C) and in CD34⁺CD38⁻ stem cells (FIG. 9D) in the BM from normal stem cells mouse models was measured by FACS analysis; no significant difference was found between the control and imetelstat treatment groups;

FIG. 9E graphically illustrates a FACS analysis and quantification of human CD45⁺ cells in BM (left panel, p=0.04 t-test) and human progenitor cells (CD34⁺CD38⁺ cells) in the BM (middle panel, p<0.05, t-test) and spleen (right panel, ns, t-test); and

FIG. 9F graphically illustrates a FACS analysis and analysis and quantification of human CD45⁺ cells in BM (left panel, p=0.04 t-test) and leukemia stem cells (CD34⁺CD38⁻ cells) in the BM (middle panel, p=0.03, t-test) and spleen (right panel, *p=0.01, t-test).

FIG. 10 illustrates supplementary Table S1.

FIG. 11 illustrates Table S2.

FIG. 12A-H illustrates data showing MPN pre-leukemia stem cell expansion and APOBEC3C activation:

FIG. 12A schematically illustrates sample distribution in the described study;

FIG. 12B graphically illustrates the mutational burden of single point mutations (log-scaled), each dot represents the number of substitutions per megabase in an individual MPN sample;

FIG. 12C graphically illustrates mutations in 69 MPN-associated genes in peripheral blood divided by MPN disease stage, clinical-grade confirmation of JAK2 V617F mutation was marked as light yellow in MPN patients;

FIG. 12D graphically illustrates a boxplot depicting the number of somatic mutations in peripheral blood or saliva based on transitions (Tis) or transversions (Tvs);

FIG. 12E graphically illustrates a boxplot depicting the expression levels of APOBEC3 in ABM, YBM, intermediate-risk myelofibrosis (Int-MF), high-risk myelofibrosis (HR-MF), and sAML stem cell populations using normalized RNA-seq.

FIG. 12F graphically illustrates a comparison of the HSC percentage in MPN samples by flow cytometry;

FIG. 12G illustrates a representative bright-field microscopic image of cord-blood CD34⁺ cells lentivirally transduced with APOBEC3C (right image) compared with a lentiviral backbone control (left image); and

FIG. 12H graphically illustrates a flow-cytometry analysis of cord-blood CD34⁺ cells 48 h after lentiviral transduction. Error bars show SEM and significance determined by two-way ANOVA,

as described in detail in Example 2, below.

FIG. 13A-H illustrate data showing that isoform switching favoring ADAR1p150 expression drives pre-LSC evolution:

FIG. 13A illustrates and image of a heatmap of RNA-seq expression of splicing isoforms for the top 1% of genes ranked by variance. Annotation for each sample is presented as a stack of colored bars representing phenotype, cell type, source tissue, mutation status, and the treatment type (for MF samples only). Samples without a known JAK2 V617F mutation status are colored in gray;

FIG. 13B graphically illustrates a boxplot representing the internally normalized expression of IL6ST isoforms and in the progenitors in each MPN phenotype, black dots represent expression values in lowest 2.5% or highest 97.5% of the distribution;

FIG. 13C graphically illustrates the ratio of ADAR1 isoforms (p150/p110) in each MPN disease type using RNA-seq expression data from stem cells and progenitors;

FIG. 13D graphically illustrates the signaling pathway impact analysis (SPIA) was performed for ET, PV, MF, and AML compared to ABM progenitors. Listed are the top 5 activated pathways based on the NDE (number of differentially expressed genes per pathway)/pSize (number of genes in the pathway) in percentages;

FIG. 13E graphically illustrates SPIA in cord blood lentivirally transduced with ADAR1 WT (top) or RNA deaminase-deficient mutant ADAR1^{E912A} (bottom) compared to pCDH backbone controls, listed are the top 6 activated pathways based on the NDE/pSize in percentages;

FIG. 13F graphically illustrates a correlation of normalized and Log 2-transformed counts per million (CPM) data for APOBEC3C with ADAR1 p150 isoform in stem cells (top) and progenitors (bottom);

FIG. 13G illustrates an image of a Western blot probed for ADAR1 p150 after co-immunoprecipitation with ADAR1 and APOBEC3C-FLAG; and

FIG. 13H illustrates an image of an Immunofluorescence showing colocalization of APOBEC3C and ADAR1 in TF1a cells: the Immunofluorescence of anti-APOBEC3C (green) and anti-ADAR1 p150-specific (red) antibodies in TF1a shADAR1 and TF1a shControl knockdown cells demonstrate a colocalization (yellow) of APOBEC3C and ADAR1 p150 proteins in the shControl cells. TF1a shADAR1 cells show ablation of ADAR1 protein,

as described in detail in Example 2, below.

FIG. 14A-G illustrate data showing that A-to-I hyper-editing distinguishes pre-LSC and LSC from normal progenitors:

FIG. 14A illustrates a Violin plot of overall RNA editing variant allele frequency (VAF) by MPN subtype and YBM and ABM controls;

FIG. 14B graphically illustrates the correlation of mean A-to-I RNA editing level with normalized and Log 2-transformed ADAR1 p150 isoform CPM level in both stem cells (square) and progenitors (triangle);

FIG. 14C graphically boxplots comparing VAF of each MPN progenitor subtype and YBM and ABM controls stratified by genomic region;

FIG. 14D illustrates a statistical comparison of data from (C). The p value values are derived from comparing the VAFs of each MPN stage and ABM at each variant classification by the Kolmogorov-Smirnov test;

FIG. 14E graphically top 25 ranked genes by occurrence of nonsynonymous RNA edit mutations broken down by known non-Alu and Alu region and previously unknown non-Alu and Alu regions stratified by MPN phenotype, treatment, and cell type;

FIG. 14F graphically illustrates normalized Log 2 transformed RNA-seq expression data for CDK13 in the stem and progenitor population plotted by MPN phenotype. The results oft tests (ns=not significant; *p<0.05; **p<0.01; ***p<0.005) between each phenotype and the ABM) group are shown;

FIG. 14G graphically illustrates expression of normalized ADAR1 RNA-seq expression data compared with expression normalized CDK13 in stem (left) and progenitor (right) populations; and

FIG. 14H illustrates an immunostaining showing colocalization of CDK13 and ADAR1 in sAML; immunostaining of cells with anti-CDK13 (green) and anti-ADAR1 (red) antibodies,

as described in detail in Example 2, below.

FIG. 15A-C illustrate data showing the RNA editome distinguishes pre-LSCs from LSCs:

FIG. 15A illustrates a heatmap based on gene expression Z scores of 1,295 differentially edited genes across all comparisons with aged bone marrow (ABM); and

FIG. 15B and FIG. 15C illustrates a network analysis of differentially edited genes between (FIG. 15B) normal aged samples (ABM) and MF and (FIG. 15C) normal aged samples (ABM) and AML,

as described in detail in Example 2, below.

FIG. 16A-H illustrate data showing ADAR1-induced STAT3 intronic editing and splice isoform switching in LSCs:

FIG. 16A illustrates a diagram of STAT3 isoform generation by intronic RNA editing of STAT3 transcripts;

FIG. 16A schematically illustrates Intronic A-to-I RNA editing locations (Goldberg et al., 2017) in ABM and AML as determined by RNA-seq analysis;

FIG. 16C upper an lower images graphically illustrate the correlation of normalized Log 2-transformed CPM data of the STAT3β isoform and the ADAR1 p150 isoform in stem cells and progenitors of ABM, YBM, MPN, and AML samples;

FIG. 16D illustrates an image of a Western blot analysis of cord-blood CD34⁺ cells (left, n=2), high-risk MF (pt. 705 and 749), and sAML (pt. 255) CD34 cells (right, n=3);

FIG. 16E illustrates an image of a Western blot analysis of sAML (pt. 255) CD34⁺ cells treated with FDA-approved JAK2 inhibitors (ruxolitinib and fedratinib) compared with a JAK3 inhibitor (FM-381) at concentrations of 1, 10, and 100 nM;

FIG. 16F graphically illustrates the correlation of ADAR1 p150 expression with the expression of STAT30 isoform;

FIG. 16G graphically illustrates pSTAT3 levels measured by flow cytometry in CD34⁺ populations of two sAML patients (2008-5 and 50261); and

FIG. 16H graphically illustrates the self-renewal capacity, as measured by colony replating assays, in MF CD34⁺ cells transduced with pCDH backbone or ADAR1 WT,

as described in detail in Example 2, below. FIG. 17A-F illustrate the top DNA mutations in MPN peripheral blood samples:

FIG. 17A illustrates Circos plots depicting single-nucleotide variants (SNVs) and structural variants (SVs) in MPN-associated samples; labels indicate the top 30 mutated genes (red font), 69 MPN-associated genes (black font);

FIG. 17B graphically illustrates mutational profiles observed after APOBEC3C expression in CD34⁺ cord blood stem cells. Mutations were detected using ensemble variant calling followed by extensive filtering of germline variants. Three types of germline filtering were used: (i) no filtering (annotated as unfiltered); (ii) filtering against all known germline variants in gnomAD and dbSNP (filtered1); (iii) filtering against all germline variants detected in CD34+ normal blood cells generated in the current study (filtered2). In all cases, the mutational profiles were consistent. Mutational profiles are shown using single base substitutions with six subtypes: C>A, C>G, C>T, T>A, T>C, T>G. Underneath each subtype are 16 bars reflecting the sequence contexts determined by the four possible bases immediately 5′ and 3′ to each mutated base.

FIG. 17C top image illustrates a Sanger sequencing analysis of APOBEC3CE68Qmutant; a G-to-C point mutation at position 68 results in an amino acid change from Glutamic Acid to Glutamine in the catalytic domain of APOBEC3C and catalytic inactivity; Bottom left: relative mRNA expression of APOBEC3C in cord blood CD34⁺ cells transduced with a lentiviral backbone (pCDH), APOBEC3C, or APOBEC3C mutant (APOBEC3CE68Q) n=3; Bottom right: brightfield images of cord blood CD34⁺ cells transduced with pCDH or APOBEC3CE68Q;

FIG. 17D graphically illustrates top mutations in MPN patients from peripheral blood including single nucleotide variants (SNVs), copy number (CN) variants and structural variants (SVs). MPN disease stage depicted in colored bar at the bottom of the figure; patient deceased since sample collection; +, patient had another malignancy; patient progressed after sample collection patient progressed to AML after sample collection; Color of alterations signifies the type of alteration. Fraction of C-to-T transitions is colored according to percent in the legend;

FIG. 17E graphically illustrates a flow gating for cord blood transduced with ADAR1 overexpressing lentiviral vectors that were engrafted; the CD19 was gated on CD45+ human cells; and

FIG. 17F graphically illustrates a colony assay with cord blood CD34⁺ cells overexpressing pCDH backbone or APOBEC3C; the self-renewal capacity as measured by relapting assay is shown on the right.

FIG. 18A-E illustrate differential gene expression in FACS-purified MPN stem cells and progenitors:

FIG. 18A illustrates images of representative gating strategy for FACS-purified stem cell (CD34+CD38−Lin−) and progenitor (CD34+CD38+Lin−) populations from 54 unique patients and 24 young and aged healthy controls;

FIG. 18B illustrates a heatmap of RNA-Seq expression of the top 1% of genes ranked by variance; annotation is shown as a stack of colored bars representing phenotype, cell type, source tissue, mutation status, and the treatment type;

FIG. 18C illustrates TF1a shADAR1 cells show ablation of ADAR1 protein by western blot. Western blot analysis of whole cell extracts (WCE) prepared from TF1a shADAR1 transduced cells with indicated antibodies confirms knockdown of ADAR1 compared with TF1a shControl cells; GAPDH is a protein loading control;

FIG. 18D illustrates a heatmap showing the top 25 differentially expressed genes in AML stem cells compared with MF stem cells (987 total DE genes); Heatmap showing the top 25 differentially expressed genes in AML progenitors compared with MF progenitors (678 total DE genes); and

FIG. 18E graphically illustrates expression of APOBEC3 family genes in FACS-purified stem cells and progenitors from normal aged (ABM), normal young (YBM), MPN and AML samples.

FIG. 19A-H illustrate differentially expressed splice isoforms and signaling pathways in MPN and AML compared with aged bone marrow stem and progenitor cells:

FIG. 19A illustrates stem and progenitor cell differential transcript expression analysis between patients with various MPN phenotypes and AML compared with aged normal bone marrow; the Venn diagram shows the overlap of significantly different genes (adjusted p-val <0.05) between the comparisons; *Adjusted Statistical significance values have been used;

FIG. 19B illustrates the top 10 statistically significantly expressed genes in the stem cell population are listed in the table;

FIG. 19C illustrates gene set enrichment analysis (GSEA) results for the comparison of AML vs MF in Stem Cells; top pathways from KEGG, REACTOME, and BIOCARTA at adj.P.Val <0.05 are shown along with the log fold change (log FC) of the pathway and the significance;

FIG. 19D illustrates the top 10 statistically significant genes in the progenitor cell population are listed in the table;

FIG. 19E illustrates GSEA results for the

comparison of AML vs MF in Progenitors; top pathways from KEGG, REACTOME, and BIOCARTA at adj.P.Val <0.1 are shown along with the log fold change (log FC) of the pathway and the significance;

FIG. 19F illustrates top enriched pathways per progenitor population by PANTHER analysis of sequencing data;

FIG. 19G illustrates a Volcano plot of stem cell population of MPN and AML compared to ABM for genes with an adjusted p-value of <0.025 (PV, AML and CML) or an adjusted p-value of <0.001 (MF); and

FIG. 19H illustrates a Volcano plot for the progenitor cell population of MPN compared to ABM for genes with an adjusted p-value of <0.001 (PV, MF and AML), and an adjusted p-value of <0.025 (CML and ET).

FIG. 20A-J illustrate A-to-I RNA editing events in MPN and AML compared with normal aged bone marrow stem and progenitor cells:

FIG. 20A illustrates correlation of normalized and Log 2-transformed CPM data for the ADAR1 p110 isoform with mean A-to-I RNA editing in stem (square) or progenitor (triangle) population of each MPN subtype and AML; each color represents a normal or disease phenotype;

FIG. 20B illustrates normalized and Log 2 transformed RNA-Seq expression data for SUMF2 in stem cells and progenitors plotted by phenotype. The results of t-tests (ns=not significant; p<0.05=*; p<0.01=**, p<0.005=***) between each phenotype and the Aged Bone Marrow (ABM); Normal group are shown;

FIG. 20C illustrates tSNE scRNA-seq analysis of cord blood CD34+ cells transduced with lentiviral backbone control or shRNA targeting ADAR1 (shADAR1);

FIG. 20D illustrates top ten differentially expressed genes between control shRNA (shCTRL) and shRNA targeting ADAR1 (shADAR1) in cord blood CD34+ cells;

FIG. 20E illustrates tSNE plot of genes expressed in scRNA-seq analysis cord blood CD34+ cells transduced with lentiviral backbone control (shCTRL) or shRNA targeting ADAR1 (shADAR1) that are found in the STRING interactome seeded with genes differentially edited between MF and aged normal bone marrow (ABM). Blue=shCTRL; red=shADAR1;

FIG. 20F illustrates tSNE plot of genes expressed in scRNA-seq analysis cord blood CD34+ cells transduced with lentiviral backbone control (shCTRL) or shRNA targeting ADAR1 (shADAR1) that are found in the STRING interactome seeded with genes differentially edited between AML and aged normal bone marrow (ABM). Blue=shCTRL; red=shADAR1;

FIG. 20G illustrates the top ten overlapping genes in MF compared with ABM and AML compared with ABM comparison that are affected by lentiviral ADAR1 shRNA knockdown;

FIG. 20H illustrates overlapping A-to-I RNA edited sites between MPN RNA-seq and cord blood overexpressing ADAR1 dataset in highly edited transcripts;

FIG. 20I illustrates the positions of RNA editing in CDK13 and STAT3 are shown; and

FIG. 20J illustrates the positions of RNA editing in SUMF2 are shown.

FIG. 21A-H illustrate ADAR1-induced STAT3 intronic editing and isoform switching in LSC:

FIG. 21A graphically illustrates expression of the STAT3 alpha and STAT3 alpha isoform levels in normal young (YBM), normal aged (ABM), and MPN and AML stem cells and progenitors using normalized and Log 2-transformed RNA-Seq analyses. (Student's t-test, p<0.05=*);

FIG. 21B graphically illustrates ADAR1, STAT3, and pSTAT3 protein levels in CD34+ primary patient samples; SDS-PAGE Western blot analysis of whole cell extract isolated from aged bone marrow (ABM, lanes 1-3), acute myeloid leukemia (AML, lanes 4 and 5), and high-risk myelofibrosis (HR-MF, lane 6) CD34+ purified cells. *, clinically treated with ruxolitinib;

FIG. 21C graphically illustrates self-renewal capacity, as measured by colony replating assays, in MF CD34+ cells transduced with pCDH backbone or an ADAR1E912A deaminase deficient mutant;

FIG. 21D graphically illustrates total number of colonies formed in primary MF CD34+ cells overexpressing ADAR1, ADAR1E912A mutant, or pCDH backbone (n=8);

FIG. 21E graphically illustrates a Western blot of whole cell extracts from TF1a parental, shCtrl and shRNA-mediated ADAR1 knockdown cells treated with or without IFN alpha;

FIG. 21F graphically illustrates a densitometry analysis of western blot of sAML (patient 255) CD34+ cells treated with FDA approved JAK2 inhibitors (ruxolitinib and fedratinib) compared with a JAK3 inhibitor (FM-381) at concentrations of 1 nM, 10 nM, and 100 nM;

FIG. 21G graphically illustrates a densitometry analysis of western blot of whole cell extracts from TF1a parental, shCtrl and shRNA-mediated ADAR1 knockdown cells treated with IFN alpha; and

FIG. 21H graphically illustrates a correlation of ADAR1 p150 expression with the expression of STAT3 alpha and CDK13; the CD34+ cells from cord blood (n=3; CB1, CB2, CB4), sAML and high-risk MF samples (n=3; 567, 705, 744) were transduced with pCDH or ADAR1 overexpressing vectors; the relative gene expression levels were measured by qRT-PCR and normalized to HPRT values.

FIG. 22A-K illustrate expression and purification of recombinant human ADAR1 Catalytic Domain (hADAR1 CD) in BJ2168 yeast expression system:

FIG. 22A illustrates a hADAR1 CD codon optimization for expression in yeast;

FIG. 22B illustrates hADAR1 CD amino acid sequence; colored amino acids: ALFDKSCSDRAMESTESRHYPVFENPKQG (SEQ ID NO:3) have been deleted in Δloop construct;

FIG. 22C illustrates pEG(KT) GST-TEV-hADAR1 CD and pEG(KT) GST-TEV-hADAR1 CD Δloop vector maps;

FIG. 22D illustrates a schematic representation of Galactose-inducible expression system;

FIG. 22E illustrates a Coomassie Blue stain and α-ADAR1 Western Blot confirming Galactose-inducible expression of GST-tagged hADAR1 CD;

FIG. 22F illustrates a workflow showing steps involved in protein purification from yeast cell extract;

FIG. 22G illustrates a Coomassie Blue stain showing successful cleavage of GST tag by TEV enzyme;

FIG. 22H illustrates a Silver stain demonstrating purity of the hADAR1 CD protein product after final purification step;

FIG. 22I illustrates a Size Exclusion Chromatography of purified hADAR1 CD using a Superdex200 10/300 GL gel filtration column; and

FIG. 22J illustrates a protein mass determination of purified hADAR1 CD protein product via mass spectrometry. (K) Analytical Ultracentrifugation of purified hADAR1 CD demonstrating purity of the final protein product,

as described in further detail in Example 3, below.

FIG. 23A-C illustrate expression and purification of recombinant human full-length ADAR1 in BJ2168 yeast expression system:

FIG. 23A illustrates a p424 10×His-tagged full-length ADAR1 vector map;

FIG. 23B illustrates a schematic representation of Galactose-inducible expression system; and

FIG. 23C illustrates a Coomassie Blue stain confirming Galactose-inducible expression of 10×His-tagged full-length ADAR1, as described in further detail in Example 3, below.

FIG. 24A-D illustrate an exemplary nano-luciferase-based RNA editase activity reporter assay in vitro:

FIG. 24A illustrates an exemplary Lentiviral NanoLuciferase RNA editase reporter expression vector;

FIG. 24B illustrates a schematic representation of an exemplary Nano-luciferase reporter design;

FIG. 24C illustrates: upper panel NanoLuciferase activity assay comparing ADAR1 RNA editase activity in K562 cells after co-transduction with pCDH/ADAR1 and NanoLuciferase reporter. lower panel α-ADAR1 Western Blot analysis demonstrating equal ADAR1 protein levels for all conditions (left) and RT-PCR showing equal expression of NanoLuciferase reporter for all conditions as well as in parental un-transduced K562 cells as a control (right); and

FIG. 24D illustrates: upper panel NanoLuciferase activity assay showing concentration-dependency and specificity for ADAR1 editase activity in HEK293T cells after co-transfection with FLAG-tagged ADAR1 constructs and NanoLuciferase reporter; lower panel α-FLAG Western Blot analysis demonstrating increasing FLAG-ADAR protein levels,

as described in further detail in Example 3, below.

FIG. 25A-D illustrates involvement of ADAR1 in the JAK/STAT pathway and JAK inhibitors as potential ADAR1-inhibiting agents:

FIG. 25A graphically illustrates ADAR1 p150 isoform expression level in TF1a cells as shown by qPCR 16 hrs after treatment with PBS (control) or Interferon alpha (normalized to HPRT);

FIG. 25B illustrates a Western Blot analysis of TF1a cells depicting protein levels of ADAR1 and various members of the JAK/STAT pathway 16 hrs after treatment with PBS (control) or Interferon alpha;

FIG. 25C illustrates a Western Blot analysis of secondary AML (patient 672) CD34+ cells showing protein levels of ADAR1, STAT3 and phospho-STAT3 Y705 16 hrs after treatment with PBS (control), interferon alpha, beta or gamma; and

FIG. 25D illustrates a Western blot analysis of secondary AML (patient 255) CD34+ cells treated with FDA approved JAK2 inhibitors (ruxolitinib and fedratinib) compared with a JAK3 inhibitor (FM-381) at concentrations of 1 nM, 10 nM, and 100 nM,

as described in further detail in Example 3, below.

FIG. 26A-D illustrate stable lentiviral shRNA-mediated knockdown of ADAR1 and stable lentiviral overexpression of ADAR1 wildtype and ADAR1 mutants after shADAR1 knockdown:

FIG. 26A graphically illustrates Total ADAR1 (left panel) and ADAR1 p150 isoform (right panel) expression levels in TF1a cells after transduction with shSchramble and shADAR1 as shown by qPCR (normalized to HPRT), confirming efficient (90%) shRNA-mediated knockdown of ADAR1;

FIG. 26B illustrates protein levels of ADAR1 in TF1a cells after transduction with shSchramble and shADAR1 as shown by Western Blot analysis, demonstrating efficient (90%) shRNA-mediated knockdown of ADAR1;

FIG. 26C illustrates Lentiviral expression vectors of HA-tagged, shADAR1-resistant (shR) ADAR1 wildtype, ADAR1 editase-deficient mutant E921A, ADAR1 DNA-binding domain-deficient mutant dZa and ADAR1 mutant E912A dZa constructs; and

FIG. 26D illustrates NanoLuciferase activity assay comparing ADAR1 RNA editase activity in TF1a cells after co-transduction with pCDH/ADAR1 shR vectors and NanoLuciferase reporter into the background of shRNA-mediated ADAR1 knockdown (left). α-HA Western Blot analysis demonstrating similar ADAR1 protein levels for all conditions,

as described in further detail in Example 3, below.

FIG. 27 illustrates result is an exemplary nano-luciferase-based RNA editase activity reporter assay in vivo: IVIS® imaging of 6.5-week-old mice after neonatal intrahepatic transplantation with K562 cells co-transduced with pCDH/wildtype ADAR1/editase-deficient ADAR1 E912A and Nano-luciferase reporter demonstrating in vivo visualization of RNA editase activity, as described in further detail in Example 3, below.

Like reference symbols in the various drawings indicate like elements.

### DETAILED DESCRIPTION

In alternative embodiments, provided are methods for treating and ameliorating a cancer such as a leukemia such as acute myeloid leukemia (AML) comprising administration to an individual in need thereof a pharmaceutical composition or a therapeutic combination of drugs comprising: imetelstat, or imetelstat, or comprising ruxolitinib, fedratinib, 8-aza-adenosine, raltegravir and/or dolutegravir or any combination thereof, and also optionally comprising second drug such as an ATP-competitive protein tyrosine kinase inhibitor such as dasatinib. In alternative embodiments, provided are methods for the in vivo inhibition of myeloproliferative neoplasm (MPN) or AML stem cell propagation comprising administration to an individual in need thereof a pharmaceutical composition comprising imetelstat, or imetelstat or comprising ruxolitinib, fedratinib, 8-aza-adenosine, raltegravir and/or dolutegravir or any combination thereof, and second drug. In alternative embodiments, provided are methods for the in vivo inhibition pre-leukemia stem cell (pre-LSC) transformation into leukemia stem cells (LSCs) comprising administration to an individual in need thereof a pharmaceutical composition comprising imetelstat, or imetelstat or comprising ruxolitinib, fedratinib, 8-aza-adenosine, raltegravir and/or dolutegravir or any combination thereof, and second drug

As supported by data provided in Example 1, combined whole genome and whole transcriptome sequencing analyses, lentiviral ADAR1 overexpression and shRNA knockdown, stromal co-culture assays and humanized pre-LSC and LSC mouse model studies reveal that pre-LSC transformation into LSC coincides with combined ADAR1, WNT/βετα-catenin and hTERT activation, which can be prevented with imetelstat.

As supported by data provided in Example 1, hTERT overexpression, ADAR1 activation and a significant reduction in telomere length correlated with accelerated stem cell aging during myeloproliferative neoplasm (MPN) progression to acute myeloid leukemia (AML). Increased ADAR1 mediated adenosine to inosine (A-to-I) transcript editing coincided with accelerated telomere shortening in high risk MPN stem cells. Treatment with imetelstat reduced MPN stem cell and LSC propagation in stromal co-cultures as well as in humanized immunocompromised mouse models commensurate with reduced hTERT expression level and telomerase activity and decreased ADAR1 editing activity.

Specifically, in vitro stromal co-culture assays revealed that combined treatment with dasatinib at 1 nM, and Iimetelstat at 1 uM or 5 uM significantly inhibited survival and replating of BC CML progenitors compared with aged bone marrow progenitors (p<0.001, ANOVA). Imetelstat (5 uM) significantly inhibited survival and replating of pre-LSC derived from myelofibrosis compared with normal bone marrow progenitor samples. In pre-LSC mouse models established from 4 different MF samples, a significant reduction in proliferation of human CD45+ cells (p<0.01, t test) was observed in bone marrow and spleen, when compared with vehicle controls. Treatment of humanized LSC mouse models, established with 5 different BC CML, with 30 mg/kg of imetelstat, three times a week for 4 weeks resulted in a significant reduction in proliferation of malignant progenitors and human CD45+ cells in blast crisis CML (p<0.001, t test, ANOVA). Following imetelstat compared with mismatch control treatment, eradication of LSC and restoration of normal hematopoiesis, was associated with normalization of telomere length as demonstrated by a FlowFish assay (p<0.05, t test, ANOVA). In addition, FACS analysis revealed a significant reduction in activated βετα-catenin expression after imetelstat treatment of LSC engrafted mice compared with vehicle controls (p<0.01, t test, ANOVA). Finally, whole transcriptome RNA sequencing analysis performed on human CD34+ cells from imetelstat treated LSC mouse models revealed a significant reduction in malignant ADAR1-mediated adenosine to inosine editing at doses that spared normal hematopoietic stem cells.

In alternative embodiments, provided are pharmaceutical compositions comprising imetelstat (Geron Corporation) as described for example in U.S. Pat. No. 9,375,485, and U.S. patent application publication nos. US 20140163090 A1; US 20150342982 A1; US 20200171072 A1; or imetelstat sodium. In alternative embodiments, provided are pharmaceutical compositions comprising imetelstat and an ATP-competitive protein tyrosine kinase inhibitor such as dasatinib (or SPRYCEL™ or DASANIX™)

Methods for formulating and administering imetelstat and ATP-competitive protein tyrosine kinase inhibitors such as dasatinib, ruxolitinib, fedratinib, 8-aza-adenosine, raltegravir and/or dolutegravir or any combination thereof, and other anti-cancer drugs, are well known in the art and can be used in methods as provided herein.

As described in Example 3, below, provided are methods of eradicating cancer cells, or cancer stem cells, comprising an ADAR1 inhibiting agent that significantly reduces ADAR1 Nano-luc reporter activity in cell lines and in human cancer stem cell assays. In alternative embodiments, the ADAR1 inhibiting agent comprises: a JAK2 inhibitor, for example, fedratinib, a STAT3 inhibitor, 8-aza-adenosine, or a nucleoside analog or integrase inhibitor such as, raltegrovir or dolutegravir, or any combination thereof.

In alternative embodiments, the ADAR1 inhibiting agent comprises a lentiviral shRNA ADAR1 knockdown vector, or a lentiviral ADAR1 mutant vector, or a lentiviral ADAR1 Z alpha domain deleted vector, or an interferon inhibitory compound.

In alternative embodiments, provided are methods to identify an ADAR1 agonist using ADAR1 Nano-luc reporter interferon responsive and interferon cell lines. In alternative embodiments, lentiviral ADAR1 or lentiviral ADAR1 shRNA are used as ADAR1 inhibiting agents. In alternative embodiments, recombinant human full length ADAR1, or recombinant human ADAR1 catalytic domain, or recombinant human Z alpha domain deleted ADAR1, is used.

In alternative embodiments, a lentiviral JAK2 overexpression vector is used.

In alternative embodiments, provided are stably transduced human non-interferon responsive cell line containing lentiviral ADAR1 overexpression vector and Nano-luc reporter for the purpose of detecting RNA virus inhibition, including SARS-CoV-2 and influenza A and B.

In alternative embodiments, provided are stably transduced human interferon responsive cell line containing lentiviral ADAR1 overexpression vector and Nano-luc reporter for the purpose of detecting RNA virus inhibition, including SARS-CoV-2, influenza A and B or HIV, following infection with an RNA virus or retrovirus.

In alternative embodiments, provided are methods of inhibiting SARs-CoV-2 or other RNA viruses or retroviruses based on lentiviral ADAR1 overexpression and intravenous (IV) administration of lentiviral ADAR1 transduced stem cells including cord blood CD34+ cells or mesenchymal stromal cells.

In alternative embodiments, provided are methods for inhibiting SARs-CoV-2 or other RNA virus or retrovirus replication based on ADAR1 catalytic domain nanoprotein delivery with liposomes by intravenous administration or inhalation.

In alternative embodiments, provide are methods inhibiting SARs-CoV-2 or other RNA virus or retrovirus replication based on ADAR1 full length nanoprotein delivery with liposomes intravenous administration or inhalation.

In alternative embodiments, provided are methods for inhibiting SARs-CoV-2 or other RNA virus or retrovirus replication based on ADAR1 Z alpha domain deleted nanoprotein delivery with liposomes intravenous administration or inhalation.

### Formulations and Pharmaceutical Compositions

In alternative embodiments, provided are pharmaceutical formulations or compositions comprising drugs, and therapeutic combinations of drugs, and formulations, and nucleic acids, vectors, recombinant viruses and liposomes, for practicing methods and uses as provided herein to treat or ameliorate a cancer, for example, to treat or ameliorate a leukemia such as acute myeloid leukemia (AML) or myeloproliferative neoplasm (MPN), or to ameliorate, protect against, reverse or decrease the severity or duration of a viral (for example, a coronavirus) infection.

In alternative embodiments, a formulation or pharmaceutical compositions used to practice methods and uses as provided herein can be administered parenterally, topically, orally or by local administration, such as by aerosol or transdermally, or intravitreal injection. The formulations and pharmaceutical compositions (including therapeutic drug combinations) can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, these compositions used to practice methods and uses as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods or uses as provided herein can be in forms such as tablets, pills, powders, capsules, liquids, gels, syrups, slurries, suspensions, etc., suitable for in vivo, in vitro or ex vivo applications.

In alternative embodiments, formulations and pharmaceutical compositions used to practice methods and uses as provided herein can comprise a solution of compositions (for example, any active agent as used in methods provided herein) disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice methods and uses as provided herein are sterile and can be manufactured to be generally free of undesirable matter. In one embodiment, these solutions and formulations are sterilized by conventional, well known sterilization techniques.

The solutions and formulations used to practice methods and uses as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compositions and formulations used to practice methods and uses as provided herein can be delivered by the use of liposomes. By using liposomes, particularly where the liposome surface carries ligands specific for target cells (for example, an injured or diseased neuronal cell or CNS tissue), or are otherwise preferentially directed to a specific tissue or organ type, one can focus the delivery of the active agent into a target cells in an in vivo, in vitro or ex vivo application.

### Nanoparticles, Nanolipoparticles and Liposomes

Also provided are nanoparticles, nanolipoparticles, vesicles and liposomal membranes comprising compounds used to practice methods and uses as provided herein, for example, to deliver compositions used to practice methods as provided herein, for example, to deliver a drug or drugs or a vector or recombinant virus, or to deliver a ADAR1 full length protein, or an ADAR1 catalytic domain, or a ADAR1 Z alpha domain deleted-protein. In alternative embodiments, these compositions are designed to target specific molecules, including biologic molecules, such as polypeptides, including cell surface polypeptides, for example, for targeting a desired cell type or organ, for example, a nerve cell or the CNS, and the like.

Provided are multilayered liposomes comprising compounds used to practice methods and uses as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods and uses as provided herein.

Liposomes can be made using any method, for example, as described in Park, et al., U.S. Pat. Pub. No. 20070042031, including method of producing a liposome by encapsulating an active agent (for example, a drug combination as provided herein, or a ADAR1-encoding nucleic acid, or a ADAR1 polypeptide), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods and uses as provided herein comprise a substituted ammonium and/or polyanions, for example, for targeting delivery of a compound (for example, a drug combination as provided herein, or a ADAR1-encoding nucleic acid, or a ADAR1 polypeptide) to a desired cell type (for example, a cancer cell), as described for example, in U.S. Pat. Pub. No. 20070110798.

Provided are nanoparticles comprising compounds (for example, a drug combination as provided herein, or a ADAR1-encoding nucleic acid, or a ADAR1 polypeptide) in the form of active agent-containing nanoparticles (for example, a secondary nanoparticle), as described, for example, in U.S. Pat. Pub. No. 20070077286. In one embodiment, provided are nanoparticles comprising a fat-soluble active agent or a fat-solubilized water-soluble active agent to act with a bivalent or trivalent metal salt.

In one embodiment, solid lipid suspensions can be used to formulate and to deliver compositions used to practice methods and uses as provided herein to mammalian cells in vivo, for example, to the CNS, as described, for example, in U.S. Pat. Pub. No. 20050136121.

### Delivery Cells and Delivery Vehicles

In alternative embodiments, any delivery vehicle can be used to practice the methods or uses as provided herein, for example, to deliver compositions (for example, a drug combination as provided herein, or a ADAR1-encoding nucleic acid, or a ADAR1 polypeptide) in vivo, to an individual in need thereof. For example, delivery vehicles comprising polycations, cationic polymers and/or cationic peptides, such as polyethyleneimine derivatives, can be used for example as described, for example, in U.S. Pat. Pub. No. 20060083737. In one embodiment, a delivery vehicle is a transduced cell engineered to express or overexpress and then secrete an endogenous or exogenous AIBP.

In one embodiment, a dried polypeptide-surfactant complex is used to formulate a composition used to practice methods as provided herein, for example as described, for example, in U.S. Pat. Pub. No. 20040151766.

In one embodiment, a composition used to practice methods and uses as provided herein can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, cells that will be subsequently delivered in vivo are transfected or transduced with an ADAR1-expressing nucleic acid, for example, a vector, for example, by electro-permeabilization, which can be used as a primary or adjunctive means to deliver the composition to a cell, for example, using any electroporation system as described for example in U.S. Pat. Nos. 7,109,034; 6,261,815; 5,874,268.

### In Vivo Delivery of AIBP-Encoding Nucleic

In alternative embodiments, provided are compositions and methods for delivering nucleic acids encoding ADAR1 peptides or polypeptides, or nucleic acids encoding peptides or polypeptides having ADAR1 activity, or vectors or recombinant viruses having contained therein these nucleic acids. In alternative embodiments, the nucleic acids, vectors or recombinant viruses are designed for in vivo or CNS delivery and expression.

In alternative embodiments, provided are compositions and methods for the delivery and controlled expression of an ADAR1-encoding nucleic acid or gene, or an expression vehicle (for example, vector, recombinant virus, and the like) comprising (having contained therein) an ADAR1-encoding nucleic acid or gene, that results in an ADAR1 protein being released into the bloodstream or general circulation where it can have a beneficial effect on in the body, for example, such as the CNS, brain or other targets.

In alternative embodiments, the provided are methods for being able to turn on and turn off ADAR1-expressing nucleic acid or gene expression easily and efficiently for tailored treatments and insurance of optimal safety.

In alternative embodiments, ADAR1 protein or proteins expressed by the AIBP-expressing nucleic acid(s) or gene(s) have a beneficial or favorable effects (for example, therapeutic or prophylactic) on a tissue or an organ, for example, the eye, or other targets, even though secreted into the blood or general circulation at a distance (for example, anatomically remote) from their site or sites of action.

In alternative embodiments, provided are expression vehicles, vectors, recombinant viruses and the like for in vivo expression of an ADAR1-encoding nucleic acid or gene to practice the methods as provide herein. In alternative embodiments, the ADAR1-encoding nucleic acids (such as RNA or DNA), expression vehicles, vectors, recombinant viruses and the like expressing the ADAR1 nucleic acid or gene can be delivered by IV, intravitreal injection or intramuscular (IM) injection (using for example, ADAR1-encoding RNA in liposomes), by intravenous (IV) injection, by subcutaneous injection, by inhalation, by a biolistic particle delivery system (for example, a so-called “gene gun”), and the like, for example, as an outpatient, for example, during an office visit.

In alternative embodiments, this “peripheral” mode of delivery, for example, expression vehicles, vectors, recombinant viruses and the like injected intravitreal, IM or IV, can circumvent problems encountered when genes or nucleic acids are expressed directly in an organ (for example, an eye, the brain or into the CNS) itself. Sustained secretion of an ADAR1 in the bloodstream or general circulation also circumvents the difficulties and expense of administering proteins by infusion.

In alternative embodiments a recombinant virus (for example, a long-term virus or viral vector), or a vector, or an expression vector, and the like, can be injected, for example, in a systemic vein (for example, IV), or by intravitreal, intramuscular (IM) injection, by inhalation, or by a biolistic particle delivery system (for example, a so-called “gene gun”), for example, as an outpatient, for example, in a physician's office. In alternative embodiments, days or weeks later (for example, four weeks later), the individual, patient or subject is administered (for example, inhales, is injected or swallows), a chemical or pharmaceutical that induces expression of the ADAR1-expressing nucleic acids or genes; for example, an oral antibiotic (for example, doxycycline or rapamycin) is administered once daily (or more or less often), which will activate the expression of the gene. In alternative embodiments, after the “activation”, or inducement of expression (for example, by an inducible promoter) of the nucleic acid or gene, ADAR1 protein is synthesized and released into the subject's circulation (for example, into the blood), and subsequently has favorable physiological effects, for example, therapeutic or prophylactic, that benefit the individual or patient (for example, benefit heart, kidney or lung function). When the physician or subject desires discontinuation of the ADAR1 treatment, the subject simply stops taking the activating chemical or pharmaceutical, for example, antibiotic.

Alternative embodiments comprise use of “expression cassettes” comprising or having contained therein a nucleotide sequence used to practice methods provided herein, for example, an ADAR1-expressing nucleic acid, which can be capable of affecting expression of the nucleic acid, for example, as a structural gene or a transcript (for example, encoding ADAR1 protein) in a host compatible with such sequences. Expression cassettes can include at least a promoter operably linked with the polypeptide coding sequence or inhibitory sequence; and, in one aspect, with other sequences, for example, transcription termination signals. Additional factors necessary or helpful in effecting expression may also be used, for example, enhancers.

In alternative aspects, expression cassettes also include plasmids, expression vectors, recombinant viruses, any form of recombinant “naked DNA” vector, and the like. In alternative aspects, a “vector” can comprise a nucleic acid that can infect, transfect, transiently or permanently transduce a cell. In alternative aspects, a vector can be a naked nucleic acid, or a nucleic acid complexed with protein or lipid. In alternative aspects, vectors can comprise viral or bacterial nucleic acids and/or proteins, and/or membranes (for example, a cell membrane, a viral lipid envelope, etc.). In alternative aspects, vectors can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, a vector can be stably replicated by the cells during mitosis as an autonomous structure, or can be incorporated within the host's genome.

In alternative aspects, “promoters” include all sequences capable of driving transcription of a coding sequence in a cell, for example, a mammalian cell such as a retinal cell. Promoters used in the constructs provided herein include cis-acting transcriptional control elements and regulatory sequences that are involved in regulating or modulating the timing and/or rate of transcription of a nucleic acid, for example, an AIBP-encoding nucleic acid. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

In alternative embodiments, “constitutive” promoters can be those that drive expression continuously under most environmental conditions and states of development or cell differentiation. In alternative embodiments, “inducible” or “regulatable” promoters can direct expression of a nucleic acid, for example, an ADAR1-encoding nucleic acid, under the influence of environmental conditions, administered chemical agents, or developmental conditions.

### Gene Therapy and Gene Delivery Vehicles

In alternative embodiments, methods of the invention comprise use of nucleic acid (for example, an ADAR1 gene or any ADAR1-encoding nucleic acid) delivery systems to deliver a payload of the nucleic acid or gene, or ADAR1-expressing nucleic acid, transcript or message, to a cell or cells in vitro, ex vivo, or in vivo, for example, as gene therapy delivery vehicles.

In alternative embodiments, expression vehicle, vector, recombinant virus, or equivalents used to practice methods provided herein are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh.10hCLN2; an organ-tropic AAV, or a neurotropic AAV; and/or an AAV capsid mutant or AAV hybrid serotype.

In alternative embodiments, the lentivirus or AAV is engineered to increase efficiency in targeting a specific cell type that is non-permissive to a wild type (wt) lentivirus or AAV and/or to improve efficacy in infecting only a cell type of interest. In alternative embodiments, the hybrid lentivirus or AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid. It is well known in the art how to engineer an adeno-associated virus (AAV) capsid in order to increase efficiency in targeting specific cell types that are non-permissive to wild type (wt) viruses and to improve efficacy in infecting only the cell type of interest; see for example, Wu et al., Mol. Ther. 2006 September; 14(3):316-27. Epub 2006 Jul. 7; Choi, et al., Curr. Gene Ther. 2005 June; 5(3):299-310.

In alternative embodiments, the ADAR1 gene or other ADAR1-encoding nucleic acid as delivered in vivo using methods as provided herein can be in the form of, or comprise, an RNA, for example, mRNA, which can be formulated in a lipid formulation or a liposome and injected for example intramuscularly (IM), for example using formulations and methods as described in U.S. patent application no. US 20210046173 A1, which describes delivering to a subject (for example, via intramuscular administration) the ADAR1 gene or other ADAR1-encoding nucleic acid that comprises a RNA (for example, mRNA) that comprises an open reading frame (ORF) that comprises (or consists of, or consists essentially of) or encodes for the ADAR1 gene or other ADAR1-encoding nucleic acid; wherein optionally the RNA (or the DNA-carrying expression vehicle) is formulated in a liposome, or a lipid nanoparticle (LNP), or nanoliposome, that comprises: non-cationic lipids comprise a mixture of cholesterol and DSPC, or a PEG-lipid, or PEG-modified lipid, or LNP, or an ionizable cationic lipid; or a mixture of (13Z,16Z)—N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9 Z)-octadec-9-en-1-yloxy]propan-2-amine; (13Z,16Z)—N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethyl amino)butanoyl)oxy) heptadecanedioate (L319), (13Z,16Z)—N,N-dimethyl-3-nonyldocosa-13,16-dien-1-amine, (12Z,15Z)—N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)—N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

Dosaging

The pharmaceutical compositions and formulations, including therapeutic drug combination, and vectors and recombinant viruses, used to practice methods and uses as provided herein can be administered for prophylactic and/or therapeutic treatments, for example, to treat or ameliorate a cancer (such as AML), or to treat, ameliorate, protect against, reverse or decrease the severity or duration of a viral infection. In therapeutic applications, compositions are administered to a subject already suffering from the cancer or viral infection in an amount sufficient to cure, alleviate or partially arrest the clinical manifestations of the disease, condition, infection or disease and its complications (a “therapeutically effective amount”), including for example, AML or a coronavirus infection. For example, in alternative embodiments, ADAR1-encoding nucleic acid- or polypeptide-comprising pharmaceutical compositions and formulations as provided herein are administered to an individual in need thereof in an amount sufficient to treat, ameliorate, protect against, reverse or decrease the severity or duration of the cancer or viral infection.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In alternative embodiments, viral vectors such as lentivirus or adenovirus or AAV vectors are administered to an individual in need therein, and in alternative embodiment the dosage administered to a human comprises: a dose of about 2×10¹² vector genomes per kg body weight (vg/kg), or between about 10¹⁰ and 10¹⁴ vector genomes per kg body weight (vg/kg), or about 10⁹, 10¹⁰, 10¹¹, 10¹², 10¹³, 10¹⁴, 10¹⁵, or more vg/kg, which can be administered as a single dosage or in multiple dosages, as needed. In alternative embodiments, these dosages are administered intravitreally, orally, IM, IV, or intrathecally. In alternative embodiments, the vectors are delivered as formulations or pharmaceutical preparations, for example, where the vectors are contained in a nanoparticle, a particle, a micelle or a liposome or lipoplex, a polymersome, a polyplex or a dendrimer. In alternative embodiments, these dosages are administered once a day, once a week, or any variation thereof as needed to maintain in vivo expression levels of ADAR1, which can be monitored by measuring actually expression of ADAR1 or by monitoring of therapeutic effect, for example, to treat, ameliorate, protect against, reverse or decrease the severity or duration of glaucoma, or neuroinflammation in an eye during glaucomatous neurodegeneration. The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations, therapeutic drug combinations, vectors or recombinant viruses can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice methods as provided herein are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1

This example demonstrates that methods as provided herein are effective for the in vivo inhibition of myeloproliferative neoplasm (MPN) or AML stem cell propagation, and for the in vivo inhibition pre-leukemia stem cell (pre-LSC) transformation into leukemia stem cells (LSCs).

Here we examined the combinatorial role of hTERT and ADAR1 in MPN pre-leukemia stem cell (pre-LSC) transformation into therapy resistant acute myeloid leukemia stem cells (LSCs) and the capacity of an oligonucleotide inhibitor of telomerase, imetelstat, to prevent transformation.

Human myeloproliferative neoplasm stem and progenitor cell (MPN-SPC) transformation into long-lived, self-renewing leukemia stem cells (LSCs) is fueled, at least in part, by ADAR1 and βετα-catenin, which transcriptionally activates telomerase reverse transcriptase (TERT). Thus, we investigated whether imetelstat, a telomerase inhibitor, prevents LSC generation.

### Methods Summary

Comparative whole genome sequencing (WGS) and whole transcriptome sequencing (RNA-seq) analyses of young, aged, MPN and AML stem and progenitor cells were performed to determine telomere content, gene expression and RNA editing activity. While confocal fluorescence microscopy was employed to assess TERT and ADAR1 localization, FlowFISH and TRAP assays were conducted to evaluate telomere length and telomerase activity. The impact of lentiviral ADAR1 wild-type and ADAR1-mutant overexpression on self-renewal gene expression in CD34⁺ cells was analyzed by RNA-seq. In MPN-SPC and LSC, beta-catenin activation was quantified by FACS. Finally, stromal co-cultures and humanized immunocompromised mouse models were established to determine imetelstat's capacity to prevent LSC generation.

### Results Summary

Reduced MPN-SPC telomere length, TERT upregulation, increased TRF2IP, and decreased AIMP2 together with ADAR1 and beta-catenin activation, marked MPN progression. Treatment with imetelstat reduced MPN-SPC and LSC propagation in vitro and in vivo, commensurate with the reduced TERT and telomerase activity. Restoration of normal hematopoiesis following imetelstat treatment, was accompanied by normalization of telomere length and reduced ADAR1 and beta-catenin activation.
WGS and RNA-seq analyses, lentiviral ADAR1 wild-type and mutant overexpression, stromal co-culture assays and humanized MPN-SPC and LSC mouse model studies all demonstrate that generation of ADAR1-activated LSCs during MPN progression can be prevented with imetelstat treatment.

### Methods—Details

### Stem and Progenitor Cell Whole Genome and Transcriptome Sequencing Analyses

Peripheral blood, bone marrow and/or saliva were obtained from consenting patients with MPNs or AML as well as young and aged healthy individuals at the University of California in accordance with Institutional Review Board-approved protocols. Human peripheral blood mononuclear cells were isolated and CD34⁺ cells were selected for stem cell whole genome sequencing (WGS) or FACS Aria purified for whole transcriptome sequencing (RNA-seq) as previously described²⁷. Stem cell (90× coverage) and saliva (30× coverage) WGS analyses were performed using Illumina HiSeq 2500 instruments for PV (n=6), ET (n=4), MF (n=26), CML (n=3) and non-MPN control individuals (n=4) following DNA extraction (see the Supplementary Appendix). Whole trancriptome (RNA-seq) was performed using Illumina Next Seq 500 sequencers using a 150-base paired-end single-index read format on RNA extracted from PV (n=6), ET (n=2), MF (n=29), CML (n=5), AML (n=12), and non-MPN (n=24) FACS-purified HSCs and HPCs, as described (see Supplementary Appendix)^{27,32}.

### Bioinformatics and Statistical Analysis

Sequencing datasets are available through dbGAP (accession number PHS002228.v1.p1). The analysis code and documentation for the computational analyses are available through Github upon request: https://github.com/ucsd-ccbb/MPN_atlas_methods. WGS Analysis: The reference genomes were realigned to the human 1000 genomes v37 or EBV. BWA-mem v.0.7.12. and WGS bioinformatics and statistical analyses were performed (see the Supplementary Appendix).

### RNA-Seq Analysis

For gene expression and RNA editing analysis, RNA editing sites were identified in REDIportal³⁴ (see the Supplementary Appendix). Genes or transcripts with an adjusted p-value of <0.05, based on the moderated t-statistic using the Benjamini-Hochberg (BH) method for multiple testing correction, were considered significantly differentially expressed (DE)³⁵. Gene Set Enrichment Analysis was performed with the Bioconductor package GSVA (see the Supplementary Appendix)³⁶. For in vitro and in vivo experiments, the Student's t-test was utilized to evaluate statistical significance of normally distributed data and Wilcoxon tests were utilized for non-normally distributed data with a p value of <0.05.

### Telomere Length and Telomerase Activity

Telomere length was determined using the Telomere PNA Kit/FITC for Flow Cytometry (Dako, Cat #K5327) with a MACSQuant10 and analyzed with FlowJo software. The Relative Telomere Length (RTL) was calculated by dividing the mean fluorescence intensity (MFI) of the test sample G0/G1 population by the MFI of the normal peripheral blood reference G0/G1 population using the Cycletest™ Plus DNA Kit (BD, Cat #340242). Telomerase activity was determined using the TRAPeze Kit RT Telomerase Detection Kit (Millipore, Catalog No. S7710).

### B-catenin Activation Analyses

A lentiviral TCF/LEF reporter was utilized in human blast crisis CML cells, K562¹. Lentiviral pCDH backbone, ADAR1^{WT}, shControl, and shADAR1 K562 stable cell lines were transduced with pGreenFire1-TCF/LEF1 reporter (SBI) lentivirus for 72-hours and analyzed using CellTiter-Glo and One-Glo luciferase assays (Promega). Mean fluorescence intensity (MFI) of activated beta-catenin in human CD45⁺ cells, HSCs (CD45⁺CD34⁺CD38⁻ Lin) and HPCs (CD45⁺CD34⁺CD38⁻ Lin) from engrafted mouse bone marrow and spleen was analyzed with a Miltenyi Biotec MACS Quant flow cytometer after staining with lineage, CD45, CD34 and CD38 (BD antibodies) as well as LIVE/DEAD stain, followed by 1% paraformaldehyde fixation, permeabilization with saponin, and staining with an anti-active beta-catenin antibody (Millipore cat #05-665, and BD custom conjugation) (see Supplementary Appendix).

### Lentiviral Overexpression and Knockdown

Lentiviral human wild-type and mutant ADAR1^{E912A} (pCDH-EF1-T2A-copGFP) and shRNA targeting ADAR1 were produced according to published protocols^{27,37}.
In Vitro MPN-SPC and LSC Survival and Self-Renewal Assays with Imetelstat
To assess MPN progenitor and LSC survival and self-renewal in vitro, human primary CD34⁺ cells selected from BC CML (n=5), MF (n=9) or age matched normal bone marrow (a-NBM, n=4) were co-cultured with SL/M2 stromal cells for 7-14 days, and then treated with imetelstat alone or combined with dasatinib for BC CML or Pacritinib for MF. Colony forming and replating assays were performed as previously described³².

### In Vivo Humanized MPN-SPC and LSC Mouse Models

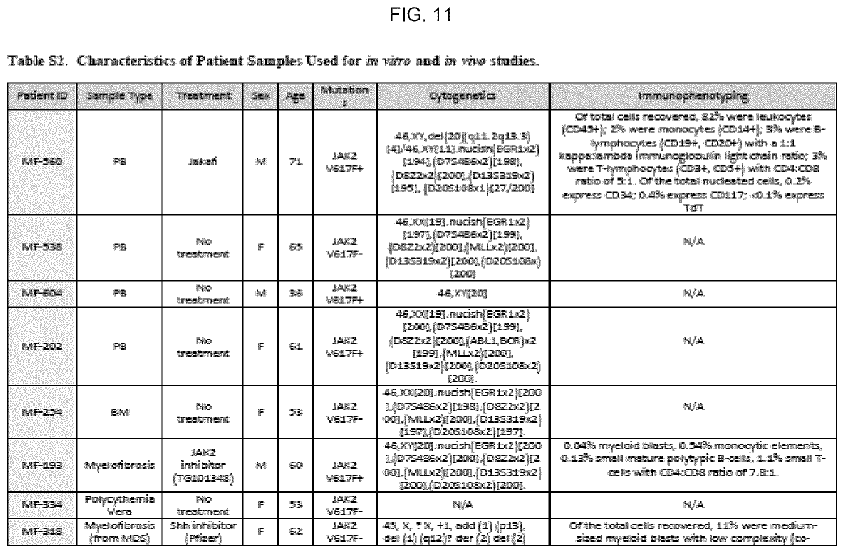

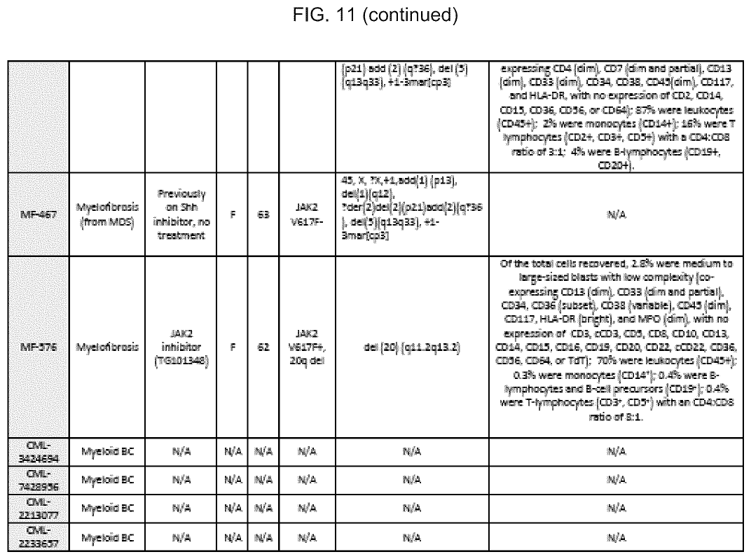

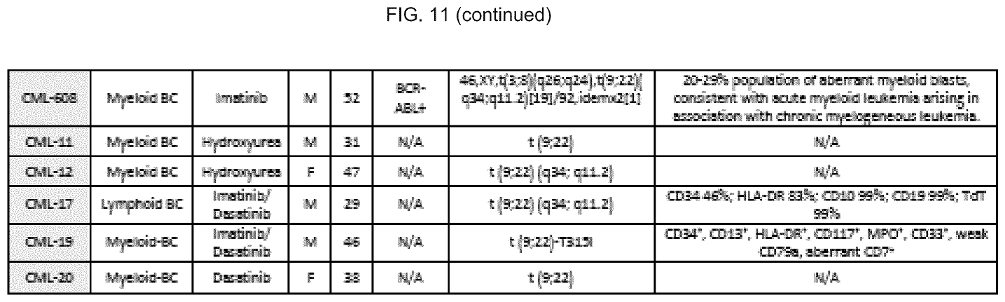

Human CD34⁺ cells isolated from MF and BC CML samples (Table S2, illustrated as FIG. 11) were transplanted intravenously into adult NSG-SGM3 (expressing human IL-3, GM-CSF and SCF) mice following 300 cGy of irradiation or intrahepatically into neonatal RAG2^{-/-} yc^{-/-} mice, respectively^{27,32}.

### DNA Extraction and Whole Genome Sequencing

Both MPN and normal peripheral blood mononuclear cells were isolated by Ficoll-paque density centrifugation followed by CD34⁺ cell selection using immunomagnetic beads according to the manufacturer's specifications (MACS; Miltenyi, Bergisch Gladbach, Germany; Catalog 130-100-453. CD34⁺ cell DNA was extracted using QIAamp DNA Blood Mini Kit (Qiagen, Catalog number 51104) according to the manufacturer's recommendations. Saliva (1 ml) was collected from the same individuals, stabilized (Biomatrica, Catalog number 97021-011A), and DNA was extracted using same method as for CD34⁺ cells. Peripheral blood (90× coverage) and saliva (30× coverage) samples were sequenced on Illumina HiSeq 2500 sequencers with a 150-base paired-end single-index read format.

### RNA Extraction and Whole Transcriptome Sequencing

Immunomagnetic bead-selected CD34+ cells were stained with fluorescent antibodies directed at human CD45, CD34, CD38 and Lineage markers (BD Pharmingen; CD2 PE-Cy5, 1:20, cat 555328, CD3 PE-Cy5, 1:20, cat 555334, CD4 PE-Cy5, 1:10, cat 555348, CD8 PE-Cy5, 1:50, cat

555368, CD14 PerCP-Cy5.5, 3:100, cat 550787, CD19 PE-Cy5, 1:50, cat 555414, CD20 PE-Cy5,

1:20, cat 555624, CD56 PE-Cy5, 1:10, cat 555517, CD45 APC, 1:50, cat 335790, CD34 BV421,

1:100, cat 562577, CD38 PE-Cy7, 1:50, cat 335790), and propidium iodide as a live dead stain. A FACS Aria II (Sanford Consortium Stem Cell Core Facility) was used to sort cells into CD45+CD34+CD38−Lin− hematopoietic stem cell and CD45+CD34+CD38+Lin− progenitor populations directly into RLT lysis buffer (Qiagen) for RNA extraction followed by RNA-Seq at The Scripps Research Institute Next Generation Sequencing Core on Illumina NextSeq 500 platforms.

| | | Catalog | | |
|---|---|---|---|---|
| Antibody | Supplier | Number | Clone | Lot |
| CD2 PE-Cy5 | BD Pharmingen | 555328 | RPA-2.10 | 6070653 |
| CD3 PE-Cy5 | BD Pharmingen | 555334 | UCHT1 | 5349958 |
| CD4 PE-Cy5 | BD Pharmingen | 555348 | RPA-T4 | 6036632 |
| CD8 PE-Cy5 | BD Pharmingen | 555368 | RPA-T8 | 5219728 |
| CD14 PerCP- | BD Pharmingen | 550787 | M5E2 | 6070674 |
| Cy5.5 | | | | |
| CD19 PE-Cy5 | BD Pharmingen | 555414 | HIB19 | 6126777 |
| CD20 PE-Cy5 | BD Pharmingen | 555624 | 2H7 | 6126778 |
| CD56 PE-Cy5 | BD Pharmingen | 555517 | B159 | 7177552 |
| CD45 APC | Life Technologies | MHCD4505 | HI30 | 1966219A |
| CD34 BV421 | BD Pharmingen | 562577 | 581 | 7153978 |
| CD38 PE-Cy7 | BD Biosciences | 335790 | HB7 | 8002648 |

### Bioinformatics and Statistical Analysis

Summary: Sequence alignment and variant calling were performed using the Genome Analysis Toolkit (GATK) best practice pipeline. We aligned the reference genomes to the human 1000 genomes v37¹, which contains the autosomes, X, Y and MT but lacks haplotype sequence or EBV. BWA-mem v.0.7.12.² was used for mapping short reads against the human 1000 genomes v37. Processing was subsequently carried out with SAMtools v.1.1³⁻⁶, Picard Tools v1.96, and Genome Analysis Toolkit (GATK) v2.4-9⁴, and consisted of the following steps: sorting and splitting of the BAM files, marking of duplicate reads, local realignment, indel realignment and recalibration of base quality scores, outputting reads coverage files in bed format for each individual, and calling germline and somatic variants. For RNA editing analysis, RNA editing sites were identified in REDIportal⁷ and RADAR⁸ as well as DARNED⁹ databases. RNA edits were annotated with Oncotator and further filtered to remove sites that exist in ExAC, 1000 Genomes Project, and dbSNP. Differential editing analysis was performed using a Chi-Square test to compare the differences in editing. To account for multiple testing, adjusted p-values were calculated using the Benjamini-Hochberg procedure and genes with events below an adjusted p-value of 0.05 were called significant. The analysis code and documentation for the computational analyses are available through Github: https://github.com/ucsd-ccbb/MPN_atlas_methods. Sequencing data sets are available through dbGAP (accession number PHS002228.v1.p1).

### Whole Genome Sequencing (WGS) Analyses

Saliva sample WGS was performed on PV (n=5), ET (n=4), MF (n=28) and CML (n=3) as well as 3 healthy volunteers and 1 patient with CLL at 30× coverage. WGS of 43 peripheral blood CD34+ stem and progenitor cell enriched samples was also performed on PV (n=6), ET (n=4), MF (n=26), CML (n=3) and 3 normal and 1 CLL sample. Sequence alignment and variant calling were performed using the Genome Analysis Toolkit (GATK) best practice pipeline with reference genomes realigned to the human 1000 genomes v37¹, which contains the autosomes, X, Y and MT but lacks haplotype sequence or EBV. To map short reads against the human 1000 genomes v37, we used BWA-mem v.0.7.12.². Then, we performed sorting and splitting of the BAM files, marking of duplicate reads, local realignment, indel realignment and recalibration of base quality scores, outputting reads coverage files in bed format for each individual, and calling germline and somatic variants with SAMtools v.1.1³⁻⁶, Picard Tools v1.96, Genome Analysis Toolkit (GATK) v2.4-9⁴.

Ensemble Variant Calling from Whole Genome Sequencing Data

Paired blood and saliva sample sequencing data were mapped against human genome build GRCh38d1.vd1 using BWA-mem v0.7.17 and sorted by SAMtools³. No minimum mapping quality score was required for mapping. Duplicate reads were annotated using Picard MarkDuplicate¹⁰ with a “STRICT” validation stringency. Variant calling was performed with GATK4 Mutect2 v4.1.4.1¹⁰, Strelka2 v2.9.10¹¹, Varscan2 v2.4.3¹², and MuSE v.1.0rc¹³. If a mutation was detected by at least 2 of the variant callers, it was considered genuine with an ensemble variant calling pipeline that was validated against ICGC PCAWG whole-genome sequenced samples exhibiting over 95% concordance in each sample¹⁴. Each of the variant callers used the gnomAD hg38 db SNP file for filtering¹⁵. With regard to Mutect2, paired reads were allowed to support different haplotypes during initial variant calling¹⁰. Contamination table and read orientation models were built from the paired samples and used for filtering. With regard to VarScan2, filtering required a minimum coverage of 10 reads and at least 3 alternative reads in CD34+ cells compared with saliva with a minimum alternative allele frequency of 0.2. For Strelka2 and MuSE, the default setting for WGS was used to produce a list of raw and filtered variants.

### Whole Transcriptome Sequencing (RNA-Seq) Analyses

For RNA-Seq, we utlized Illumina NextSeq 500 sequencers with 150 bp paired-end reads. Sequencing data were obtained as Illumina bcl2fastq (v2.17) files.

### A-to-I RNA Editing Analysis

To quantify adenosine-to-inosine (A-to-I) RNA editing events induced by ADAR1, RNA reads were aligned using 2-pass alignment with STAR 2.5.2b 2-pass alignment. Alignment deduplication was performed with Picard MarkDuplicates and SortSam, and further processed according to GATK best practices for calling RNA-Seq variants with tools SplitNCigarReads, RealignerTargetCreator, IndelRealigner, BaseRecalibrator, PrintReads. Variants were called with HaplotypeCaller and filtered with VariantFiltration for FS<30, QD>2, QUAL >20(79). Alu sites were identified and kept from RepeatMasker. Non-Alu variants were removed in repetitive regions based on the RepeatMasker annotation. Intronic sites within 4 bp of splicing junctions were removed. All sites were kept if a minimum of three alternative allele carrying reads and ten total reads and a minimum allele frequency of 0.10 were present. We identified known RNA editing sites according to REDIportal⁷, RADAR⁸ and DARNED⁹. We kept A-to-G variants in genes on the positive strand and T-to-C variants on the negative strand in order to filter mismatches to ADAR-specific RNA edits^{8,9,16-18}. We only predicted RNA editing sites with >10 reads/site to compare mismatches and excluded sites that were identified as DNA variants. RNA edits were annotated with Oncotator and filtered to remove sites that exist in ExAC, 1000 Genomes Project, and dbSNP. Differential editing analysis was performed using a Chi-Square test to compare the differences in editing in each gene for each variant classification (i.e. MDM2-3′UTR MF vs AN). Significance was set at p<0.05. To account for multiple testing, adjusted p-values were calculated using the Benjamini-Hochberg procedure and genes with events below an adjusted p-value of 0.05 were called significant and retained in the final lists.

### Transcript Differential Expression

FastQC (Andrews, S. & Others. FastQC: a quality control tool for high throughput sequence data. (2010) was used to perform quality control on raw fastq files. Sequencing reads were aligned to the human genome (hg19) using the STAR v2.5.1a aligner¹⁹. RSEM²⁰ v1.3.0 and GENCODE annotation (genocode.v19.annotation.gtf) were used for read and transcript quantification, and R BioConductor packages edgeR²¹ and Limma²² were used to implement the limma-voom²³ method for differential expression analysis at both the gene and transcript levels. The experimental design was modeled upon disease and tissue type (˜0+disease; ˜0+tissue; ˜0+disease+tissue). Significance was defined by using an adjusted p-value cut-off of 0.05 after multiple testing correction using a moderated t-statistic in Limma. Genes or transcripts with an adjusted p-value of <0.05 (based on the moderated t-statistic using the Benjamini-Hochberg (BH) method for multiple testing correction) were considered significantly differentially expressed (DE)²⁴. Gene Set Enrichment Analysis was performed with the Bioconductor package GSVA²⁵.

For in vitro and in vivo experiments, the Student's t-test was utilized to evaluate statistical significance of normally distributed data and Wilcoxon tests were utilized for non-normally distributed data with a p value of <0.05.

### Confocal Fluorescence Microscopy

For immunofluorescence, cells (4×10⁵ cells/ml) incubated in ice-cold buffer (10 mM PIPES, pH 6.8; 100 mM NaCl; 300 mM sucrose; 3 mM MgCl2) for 1 min and then transferred into ice-cold CSKT buffer incubate for 5 min, followed by ice-cold CSK buffer for 1 min and then 4% paraformaldehyde in PBS for 10 min at room temperature. Immunofluorescence was performed by immersing slides in PB ST (1×PBS with 0.1% Tween-20) for several minutes. Slides were overlaid with 250 microliters of blocking solution (1×PBS, 1% fetal bovine serum, 0.1% Tween-20) for 1 hour at room temperature. Blocking solution was removed and 100 microliters of primary antibody was added to the cells and incubated for 3 hours at room temperature. The slides were washed 2 times with PB ST for 5 min each at room temperature. Secondary antibody was overlaid to spotted cells for 1 hour in the dark. Slides were washed with PBST 2× at room temperature. DAPI was added and the slides were sealed with a coverslip. Imaging was performed using an Olympus Fluoview confocal microscope.

### Telomeric Length and Telomerase Activity

Samples were selected from engrafted mouse bone marrow or spleen with human CD45+ immunomagnetic beads (Miltenyi). Telomere length was determined by using the Telomere PNA Kit/FITC for Flow Cytometry (Dako, Cat #K5327). Samples were run on the MACSQuant10 and analyzed with FlowJo software. The Relative Telomere Length (RTL) was calculated. The 1301 cell line was utilized as control cells. The DNA index was determined with the (BD Cycletest™ Plus DNA Kit, Cat #340242). The DNA index was obtained by dividing the MFI of the test sample G0/G1 population by the WI of the normal reference G0/G1 population. Human peripheral blood mononuclear cells were used as “the normal reference G0/G1 population” for calculating the DNA index. Telomerase activity was determined by using TRAPeze Kit RT Telomerase Detection Kit (Millipore, Catalog No. S7710). Cell lysates from 5×10⁵ CD34⁺ selected cells from BC CML engrafted mice or K562 cells were prepared according to the manufacturer's protocol. The reactions were performed in triplicate and a TSR8 standard curve was generated using dilutions of a TSR8 internal control template. Arbitrary telomerase activity units were extrapolated from the standard curve using the experimental average Ct values.

beta-catenin Activation Analyses

To assess WNT/beta-catenin activation in response to ADAR1 upregulation, a lentiviral TCF/LEF reporter was utilized in human blast crisis CML cells, K562²⁶. Lentiviral pCDH backbone, ADAR1^{WT}, shControl, and shADAR1 K562 stable cell lines were transduced with pGreenFire1-TCF/LEF1 reporter (SBI) lentivirus for 72-hours prior to CellTiter-Glo and One-Glo luciferase assays (Promega). A minimum of 50,000 cells were plated in duplicate, lysed according to the manufacturer's protocol, and measured using preloaded protocols on a Promega GloMax Discover Microplate Reader. One-Glo luciferase readouts were normalized to corresponding CellTiter-Glo values. Activated beta-catenin levels in HSC and HPCs were also quantified using a MACS Quant instrument following staining of cells with Viability Near-IR, Lineage PE-Cy5, CD45 BV510, CD34 BV421, CD38 PE-Cy7, GFP, and beta-catenin AF647 antibodies (Becton Dickinson, Inc). Specifically, FACS analysis was performed on a minimum of 200,000 cells that were subjected to FcR blocking using anti-human (Miltenyi Biotec cat #130-059-901) and anti-mouse FcR blocking reagents (BD cat #553142) and then stained with ethidium monoazide (Life Technologies cat #E1372) or LIVE/DEAD Fixable Dead Cell Stain (Invitrogen cat #L34975), anti-lineage antibody cocktail (CD2 PE-Cy5 BD cat #555328, CD3 PE-Cy5 BD cat #555334, CD4 PE-Cy5 BD cat #555348, CD8 PE-Cy5 BD cat #555368, CD14 PerCP-Cy5.5 BD cat #550787, CD19 PE-Cy5 BD cat #555414, CD20 PE-Cy5 BD cat #555624, CD56 PE-Cy5 BD cat #555517), CD45 BB515 (BD cat #564585), CD34 BV421 (BD cat #740081), and CD38 PE-Cy7 (BD cat #335790). Cells were then fixed with 1% paraformaldehyde, permeabilized with dilute saponin, and stained with anti-active beta-catenin AF647 (Millipore cat #05-665, and BD custom conjugation). Mean fluorescence intensity (MF) of active beta-catenin in human CD45+ cells, HSCs (CD45⁺CD34⁺CD38⁻ Lin) and HPCs (CD45⁺CD34⁺CD38⁺Lin⁻) derived from engrafted bone marrow and spleen was determined with a Miltenyi Biotec MACS Quant flow cytometer.

### Lentiviral Overexpression and Knockdown

Lentiviral human wild-type and mutant ADAR1^{E912A} (pCDH-EF1-T2A-copGFP) and shRNA targeting ADAR1 were produced according to published protocols^{27,28}. All lentiviruses were tested by transduction of 293T cells and transduction efficiency was assessed by qRT-PCR. Lentiviral transduction of primary patient samples was performed at a MOI of 100 to 200. The cells were cultured for 72 hours in 96-well plates (2×10⁵-5×10⁵ cells per well) containing StemPro (Life Technologies) media supplemented with human IL-6, stem cell factor (SCF), Thrombopoietin (Tpo) and FLT-3 (all from R&D Systems)^{27,29-31}. The transduced cells were collected for RNA extraction and cDNA was synthesized according to published methods^{27,29-31}.

In Vitro MPN-SPC and LSC Survival and Self-Renewal Assays with Imetelstat

To assess MPN progenitor and LSC survival and self-renewal in vitro, human primary CD34⁺ cells selected from BC CML (n=5), MF (n=9) or age matched normal bone marrow (a-NBM, n=4) were co-cultured with SL/M2 stromal cells for 7-14 days, and then treated with Imetelstat alone or combined with dasatinib for BC CML or Pacritinib for MF. Colony forming and replating assays were performed as previously described^{32,33}.

### MPN-SPC and LSC Humanized Mouse Models

Human CD34⁺ cells isolated from MF and BC CML (Table S2) samples were transplanted intravenously into adult NSG-SGM3 (expressing human IL-3, GM-CSF and SCF) mice following

300 cGy of irradiation or intrahepatically into newborn RAG2^{-/-}□c^{-/-} mice^{28,32,34}. Post-transplantation (13 to 16 weeks), bone marrow and spleen were collected and analyzed by FACS³². Mice were treated with vehicle, mismatch or imetelstat at varying doses. Also, MF CD34⁺ cells were transduced with pCDH lentivirus control or ADAR1-OE lentivirus with a MOI of 100 for 48 hours, followed by intravenous transplantation into NSG-SGM3 mice to assess the impact of ADAR1 overexpression on MF engraftment.

### Results—Details

### Hematopoietic Stem and Progenitor Cell Telomere Shortening Predicts MPN Progression

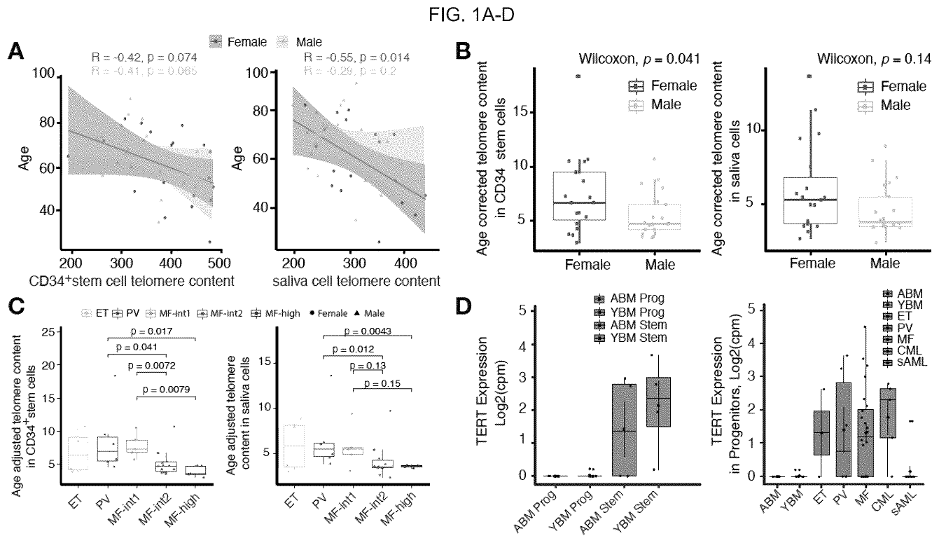

Comparative WGS analyses of intratelomeric reads per million reads in 38 MPN and non-MPN sample-derived CD34⁺ hematopoietic stem and progenitor cells and matching bulk saliva revealed the age dependency of telomere length in stem cell-enriched populations (Table S1 and FIG. S1). As expected, we found that telomeres shortened during the process of aging in differentiated cells in saliva as well as in CD34⁺ hematopoietic stem cells (FIG. 1A). While no significant differences were detected in saliva-derived cells (p=0.14), age-corrected telomere length was significantly reduced in stem cell enriched populations (p=0.041) derived from males compared with females (FIG. 1B). Moreover, age-corrected telomere shortening was more prominent in MPN stem and progenitor cells (MPN-SPC) than saliva during progression (FIG. 1C). Furthermore, significant telomere shortening marked disease progression in myelofibrosis (MF) CD34⁺ stem cells (p=0.0079) rather than bulk saliva (p=0.15) cells (FIG. 1C). As determined by RNA-seq, TERT expression was highest in young bone marrow (YBM) HSC, decreased in aged bone marrow (ABM) HSC and was barely detectable in YBM or ABM progenitors (FIG. 1D). In contrast to normal progenitors, MPN progenitors harbored high levels of TERT expression compared with their normal counterparts (FIG. 1D). Thus, telomere shortening, regulated via differential expression of TERT, is not only related to aging, but appears to be an important mechanism governing MPN progression.

ADAR1 Activation Correlates with Beta-Catenin and hTERT Upregulation in MPN-SPC

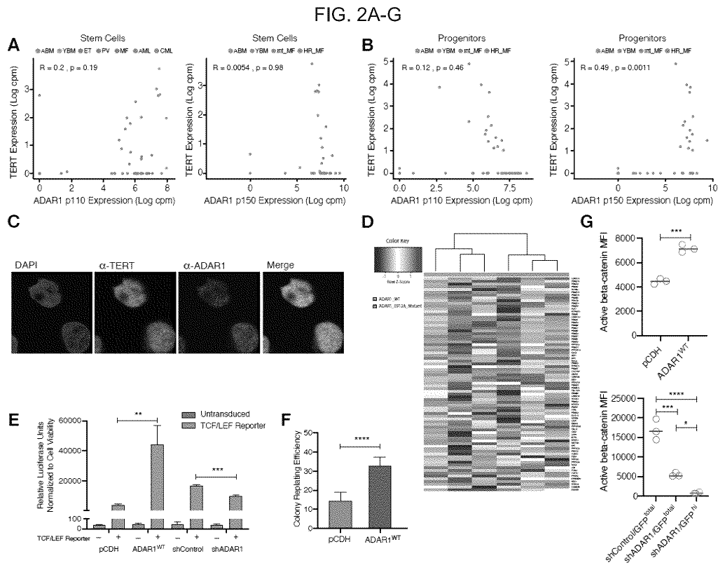

To further investigate mechanisms associated with TERT overexpression, RNA-seq was performed on young, aged, and MPN stem (FIG. 2A) and progenitor cells (FIG. 2B). Compared with aged normal bone marrow, intermediate and high risk MF progenitors exhibited higher levels of both ADAR1p150 and TERT (R=0.49, p=0.001) (FIG. 2B). Confocal fluorescence microscopy revealed co-localization of ADAR1 and TERT in TF1a leukemia cells (FIG. 2C). Moreover, lentiviral overexpression of ADAR1 wild-type compared with an A-to-I editing defective mutant in cord blood CD34⁺ cells, revealed upregulation of multiple WNT/beta-catenin self-renewal pathway activating genes (FIG. 2D). Compatible with ADAR1 activation of beta-catenin signaling, lentiviral ADAR1 wild-type overexpression in K562 leukemia cells increased (p<0.01) while lentiviral shADAR1 knockdown reduced TCF/LEF-luciferase reporter activity compared with pCDH controls (p<0.001) (FIG. 2E). Additionally, lentiviral ADAR1 WT transduction enhanced myelofibrosis (MF) CD34⁺ cell replating capacity compared with pCDH backbone controls (p<0.001) (FIG. 2F). While FACS analysis demonstrated that lentiviral ADAR1p150 overexpression increased activated beta-catenin (p<0.001) in leukemia cells, lentiviral ADAR1 shRNA knockdown reduced beta-catenin (p<0.001) compared with pCDH controls (FIG. 2G). Together, these data suggest that ADAR1 induces beta-catenin self-renewal pathway activation in MF progenitors with enhanced longevity due to TERT expression.

### Imetelstat Prevents MPN-SPC and LSC Maintenance In Vitro

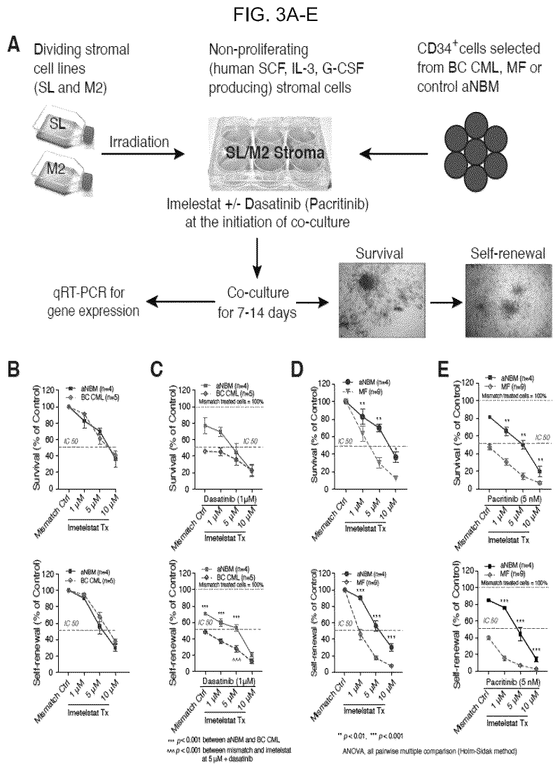

To investigate whether inhibition of telomerase activity in MPN progenitors and LSC inhibited their survival and self-renewal, we performed stromal co-culture experiments using imetelstat (Geron, Inc) compared with mismatch and vehicle controls (FIG. 3A). While imetelstat monotherapy was ineffective at reducing BC CML LSC replating (self-renewal) capacity (FIG. 3B) as well as ADAR1 and the beta-catenin self-renewal gene expression by qRT-PCR (Figure S2A), the LSC replating was reduced by combined treatment with imetelstat and Dasatinib (p<0.001) compared with aged normal bone marrow (aNBM) controls (FIG. 3C). In MF CD34⁺ compared with normal bone marrow cells, a decrease in both survival (p<0.01) and self-renewal capacity (p<0.001) was observed with imetelstat only treatment (FIG. 3D). Combination treatment with imetelstat and Pacritinib, a low-dose JAK2 inhibitor, did not appear to synergize significantly with imetelstat in MF (FIG. 3E). Based on these data, imetelstat prevents MPN-SPC transformation into LSCs.

### Telomerase Inhibition Prevents MPN-SPC and LSC Maintenance In Vivo

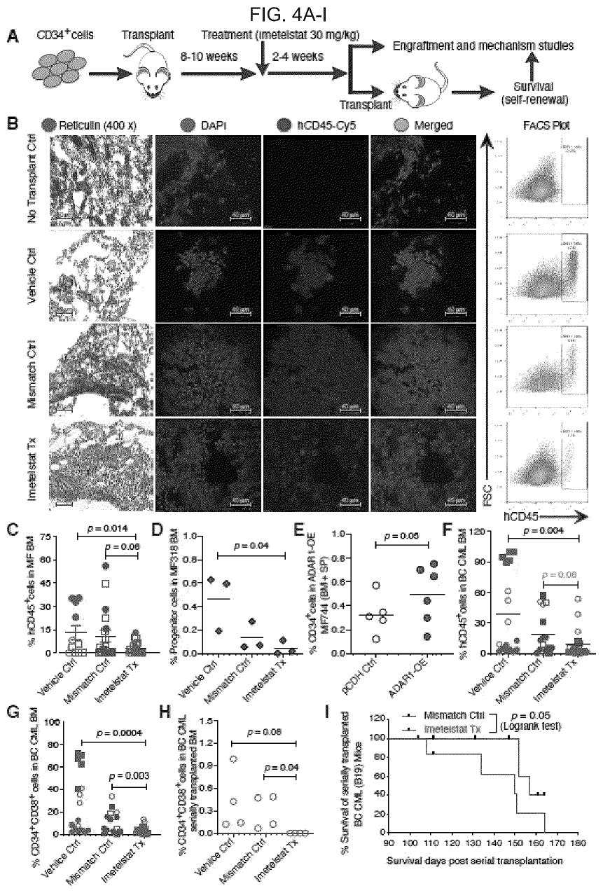

Next, we investigated the impact of imetelstat treatment compared with mismatch and vehicle control treatment on LSC and MPN-SPC maintenance in humanized BC CML and MF mouse models, respectively (FIG. 4A, Table S2). Immunohistochemistry analysis showed that bone marrow from imetelstat-treated mice harbored less reticulin fibrosis and had lower human CD45⁺ leukemic cell engraftment, by confocal immunofluorescence microscopy analysis, than vehicle and mismatch controls (FIG. 4B). Furthermore, FACS analysis revealed lower human CD45+ (p<0.014) and progenitor (p<0.04) engraftment in imetelstat compared to control treated mice commensurate with decreased splenomegaly (FIGS. 4B, 4C and 4D, Figure S3). Primary MF CD34⁺ cells that were lentivirally transduced with ADAR1 had higher engraftment compared to controls, compatible with the hypothesis that ADAR1 drives disease propagation (FIG. 4E). Moreover, imetelstat treatment reduced human CD45⁺ and progenitor engraftment in CML BC engrafted mice compared to vehicle controls (FIGS. 4F and 4G). Subsequently, we performed serial transplantation as the gold standard assay to evaluate LSC self-renewal capacity. We observed significantly less LSC engraftment (p=0.04) after imetelstat treatment, compared with mismatch control treatment (FIG. 4H) and longer survival rates (p=0.05) (FIG. 4I). Thus, imetelstat treatment inhibits both MPN-SPC and LSC maintenance in vivo.

Telomerase Inhibition with Imetelstat Prevents ADAR1-Activated LSC Propagation

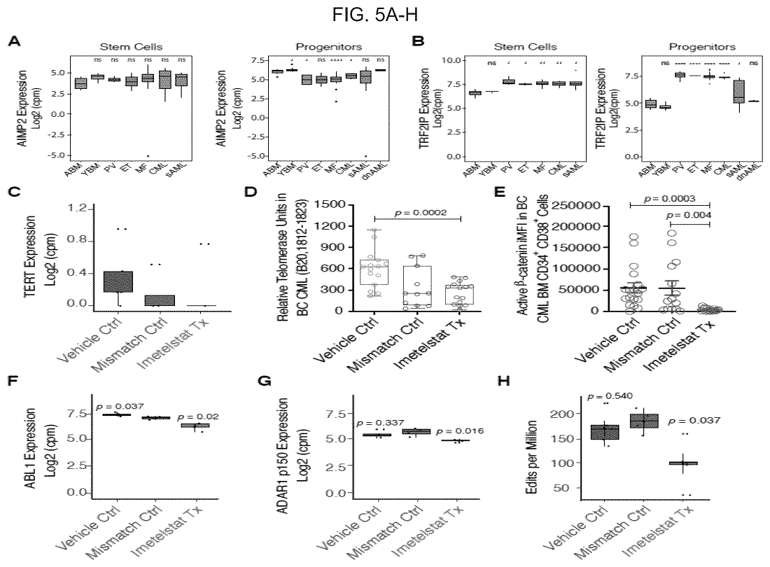

To further investigate the mechanisms governing LSC maintenance, RNA-seq was performed on FACS purified stem cells and progenitors from young and aged normal bone marrow, PV, ET, MF, CML, sAML and de novo AML (dnAML) samples. Compatible with ADAR1 activation during MPN progression, transcript levels of AIMP2, a proteolytic degrader of ADAR1, were significantly reduced in MF progenitors (p<0.001) compared with aged bone marrow controls (FIG. 5A). In addition, levels of TRF2IP, which binds to AIMP2 and has been implicated in cancer progression, were significantly increased in PV, ET, MF, CML and sAML compared with aged bone marrow progenitors (FIG. 5B). In imetelstat compared with vehicle treated humanized BC LSC models, RNA-seq analysis of surviving human CD45⁺ cells demonstrated a reduction (p<0.01) in TERT (FIG. 5C) as well as telomerase activity (p<0.0002) as quantified by TRAP assay (FIG. 5D). Moreover, imetelstat treatment significantly reduced beta-catenin activity (p=0.0003) in LSC as quantified by FACS analysis (FIG. 5E); ABL1 (p=0.02) and ADAR1p150 expression (p=0.016) as measured by qRT-PCR (FIGS. 5F and 5G); and ADAR1-mediated RNA editing (p=0.037) as quantified by RNA-seq (FIG. 5H). Although there was no significant decrease in normal cord blood engraftment or beta-catenin expression following imetelstat treatment in vivo (Figure S5), LSCs were consistently therapeutically vulnerable to imetelstat.

### Discussion

While telomerase activation has been linked to cancer progression and therapeutic resistance, telomerase inhibition with imetelstat has shown considerable promise in ET and MF clinical trials^{6,7,11,16}. However, the mechanism of action with regard to curbing MPN progression has not been clearly elucidated. As a scaffolding ribonucleoprotein complex, telomerase contains human telomerase RNA (hTR), TERT, and cofactors¹⁷. The template region of hTR binds to the telomeric 3′ end and the telomere repeats are bound by the Shelterin complex. Double-strand DNA binding proteins, including TRF1 and TRF2, bind to telomeric DNA. Subsequently, TRF2 binds to TRF2IP (also known as RAP1). Both TRF1 and TRF2 interact with TRF1-interacting nuclear factor 2 (TIN2) that binds to TPP1, which also binds protection of telomeres 1 (POT1), thereby recruiting telomerase to telomeres via the N terminal TERT domain.

Of particular relevance to this study, TRF2IP binds to AIMP2, which is a proteolytic degrader of ADAR1 and thus may regulate ADAR1 activity²³⁻²⁵. Indeed, we discovered significant downregulation of AIMP2 and increased TRF2IP expression in MPN progenitors, which could sequester remaining AIMP2 and thus contribute to ADAR1 activation in MPN-SPCs and LSCs. Moreover, by binding to its co-factors, telomerase acquires an RNA-tethered two lobed structure²², which could conceivably be targeted by inflammation-induced APOBEC3 and ADAR1 deaminases in malignant microenvironments. In many cancers, C-to-T transitions at 124 bp (C228T) and 146 bp (C250T) upstream of the TERT translation start codon create additional ETS transcription factor binding sites resulting in TERT transcription and telomerase activation²². Recently, we and others showed that activation of primate-specific APOBEC3 C-to-T deaminase enzymes episodically induce maligant mutagenesis³⁸. Moreover, APOBEC3C overexpression is associated with increased C-to-T transitions and cancer progression (Alexandrov). It is conceivable that inflammatory-cytokine induced APOBEC3C activation induces the C-to-T transitions as an inflammation induced mechanism capable of enhancing ETS binding and TERT upregulation in the MPN bone marrow microenvironments.

By employing whole genome sequencing analysis of hematopoietic stem and progenitor cells, we discovered that MPN progression is associated with a significant reduction in telomere length, which is more prominent in male compared with female patients. While accelerated telomere shortening has been linked with other malignancies, this is the first description of telomere shortening during MPN progression at the stem cell level. Moreover, whole transcriptome sequencing demonstrated that MPN progression was associated with both TERT and inflammatory-cytokine inducible ADAR1p150 expression as well as AIMP2 downregulation and TRF2IP upregulation^{23,31,39,40}. Lentiviral ADAR1 overexpression fueled beta-catenin activation in CD34⁺ cells and increased MF CD34⁺ cell replating, indicative of enhanced self-renewal capacity. In addition, ADAR1 shRNA knockdown reduced both ADAR1 and TERT protein expression. Thus, ADAR1 activates beta-catenin, which is known to bind to the TERT promoter and activate transcription thereby linking ADAR1 and telomerase activation. Finally, a selective telomerase inhibitor, imetelstat, inhibits both MPN-SPC and LSC maintenance in stromal co-cultures and in humanized mouse models commensurate with a reduction in beta-catenin and ADAR1 activity as well as normalization of telomere activity. In conclusion, imetelsat prevents MPN-SPC progression to ADAR1-activated LSC and thus may obviate therapeutic resistance-induced AML transformation

### FIGURE LEGENDS

FIG. 1. Quantification of Telomerase Activation in MPN Stem and Progenitor Cells (MPN-SPC)

FIG. 1A. Age dependency of telomere content in CD34⁺ stem cells (female: p=0.074, male: p=0.065) and bulk saliva (female: p=0.014, male: p=0.2) was quantified by whole genome sequencing (WGS) analysis. CD34⁺ cell whole-genome sequencing (90×) was performed on PV (n=6), ET (n=4), MF (n=26), CML (n=3) and non-MPN control individuals (n=4) with matching saliva (30× coverage). X-axes reflect telomere context, measured by the number of intratelomeric reads per million reads after GC content correction; y-axes depict age at time of diagnosis. Gray area reflects 95% confidence intervals of regression line.

FIG. 1B. Comparative WGS analysis between age adjusted telomere lengths in males and females in both CD34⁺ stem cells (p=0.041) and bulk saliva (p=0.14). Boxplots depict the distribution of age adjusted telomere lengths for males and females.

FIG. 1C. WGS analysis comparison of age adjusted telomere lengths during MPN progression in CD34⁺ stem cells and bulk saliva. Boxplots depict the distribution of age adjusted telomere lengths for different stages of MPN.

FIG. 1D. Left: A boxplot depicting TERT expression in stem cells and progenitors that were FACS-purified from normal young (YBM) and aged (ABM) bone marrow and subjected to RNA-seq analysis. Right: TERT gene expression levels by RNA-seq for ABM and YBM (n=24), ET (n=2), PV (n=6), MF (n=29) and sAML (n=12) progenitors. RNA-seq results are shown as normalized, log 2 transformed counts per million (Log 2cpm).

FIG. 2. ADAR1p150, beta-catenin and hTERT Upregulation Characterize MPN-SPC

FIG. 2A. Left: Correlation of normalized log transformed counts per million (Log cpm) RNA-seq expression data for TERT and ADAR p110 isoform in FACS purified stem cells from aged bone marrow (ABM, red), young bone marrow (YBM, yellow), essential thrombocythemia (ET, green), polycythemia vera (PV, teal), acute myeloid leukemia (AML, pink) and chronic myeloid leukemia (CML, purple) (p=0.19). Right: TERT and ADAR1 p150 expression levels (Log cpm) in stem cells that were FACS purified from aged bone marrow (ABM, red), young bone marrow (YBM, green), intermediate MF (int-MF, blue) and high risk MF (HR MF, purple) (p=0.98).

FIG. 2B. Correlation of normalized log transformed counts per million (Log cpm) RNA-seq expression data for TERT and ADAR p110 isoform (left, p=0.46) or TERT and ADARp150 isoform (right, p=0.0011) in progenitors that were FACS-purified from aged bone marrow (ABM, red), young bone marrow (YBM, green), intermediate MF (int-MF, blue) and high risk MF (HR MF, purple).

FIG. 2C. Confocal fluorescence photomicrographs depicting DAPI (blue, far left), TERT (green, middle left), ADAR1 (red, middle right) and merged images (right) of TERT and ADAR1 localization (yellow).

FIG. 2D. Hierarchical clustering gene expression analysis of RNA-seq data derived from CD34⁺ cord blood cells that were lentivirally transduced with ADAR1 wild-type (ADAR1 WT, orange) or a deaminase defective mutant (ADAR1 E912A Mutant, green).

FIG. 2E. TCF/LEF reporter activity was measured by firefly luciferase and normalized to cell viability in K562 cells lentivirally transduced with pCDH backbone, ADAR1^{WT}, shControl or shADAR1. Cells were either singly transduced with ADAR1 OE and shRNA constructs or co-transduced with TCF/LEF reporter. **p=0.0052, ***p=0.0002.

FIG. 2F. Self-renewal capacity was measured by colony replating assays in primary myelofibrosis CD34⁺ cells lentivirally transduced with either pCDH backbone (grey bar) or ADAR1^{WT} (red bar) (Student's t-test, p<0.0001).

FIG. 2G. Upper FACS plot: FACS analysis of mean fluorescence intensity (MFI) of active (non-phosphorylated) beta-catenin levels in BC CML leukemia (K562) cells transduced with lentiviral-GFP vectors expressing ADAR1 (ADAR1^{WT}; red circles or pCDH backbone (blue circles) (Student's t-test, p=0.0003). Lower FACS plot: FACS analysis of beta-catenin MFI in KG1a CD34⁺ leukemia cells transduced with shScramble control compared with shADAR1 lentiviral vectors. Stati (p<0.0001 by one-way ANOVA. Tukey's multiple comparisons post-hoc tests yielded adjusted p-values of 0.0003 between shScramble (blue) and shADAR1-GFP-total (red); <0.0001 between shScramble control (blue) and shADAR1-GFP-hi (green); and 0.0224 between shADAR1-GFP-total (red) and shADAR1-GFP-hi (green)).

FIG. 3. Telomerase Inhibition Prevents MPN-SPC and LSC Maintenance In Vitro

FIG. 3A. A schematic illustration of SL/M2 stromal co-cultures established to quantify in vitro MPN-SPC and LSC survival and self-renewal. Human primary CD34⁺ cells selected from BC CML (n=5), MF (n=9) or age matched normal bone marrow (a-NBM, n=4) were co-cultured with SL/M2 stromal cells for 7-14 days. While BC CML stromal co-cultures were treated with increasing doses of imetelstat with or without dasatinib, MF stromal co-cultures were treated with imetelast with or without pacritinib.

FIG. 3B. Imetelstat was used at concentrations ranging from 1 to 10 μM and compared with mismatch controls in aged normal bone marrow (aNBM, n=4, blue) and the BC CML (n=5, red) stromal co-cultures followed by clonogenic survival (upper panel) and self-renewal (lower panel) assays.

FIG. 3C. Imetelstat was used at concentrations ranging from 1 to 10 μM and compared with mismatch controls in aged normal bone marrow (aNBM, n=4, blue) compared with BC CML (n=5, red) stromal co-cultures followed by clonogenic survival (upper panel) and self-renewal (lower panel) assays.

FIG. 3C. Imetelstat was used at concentrations ranging from 1 to 10 μM alone or in combination with dasatinib (1 μM) to treat aged normal bone marrow (a-NBM, n=4, black) compared with BC CML (purple, n=5) in stromal co-cultures followed by clonogenic survival (upper panel) and self-renewal (lower panel) assays (**p<0.01, ***p<0.001).

FIG. 3D. Imetelstat was used at concentrations ranging from 1 to 10 μM to treat aged normal bone marrow (aNBM, n=4, dark blue) compared with myelofibrosis (MF, light blue, n=9) in stromal co-cultures followed by clonogenic survival (upper panel) and self-renewal (lower panel) assays (**p<0.01, ***p<0.001).

FIG. 3E. Imetelstat was used at concentrations ranging from 1 to 10 μM alone or in combination with pacritinib (5 nM) to treat aged normal bone marrow (a-NBM, n=4, blue) compared with MF (green, n=9) in stromal co-cultures followed by clonogenic survival (upper panel) and self-renewal (lower panel) assays.

FIG. 4 Telomerase Inhibition Prevents MPN-SPC and LSC Maintenance In Vivo

FIG. 4A. A schema of humanized BC CML LSC and MF SPC mouse models. Human CD34⁺ cells sorted from either BC CML or myelofibrosis patient blood or marrow were transplanted into neonatal Rag2^{-/-}□c^{-/-} mice or intravenously transplanted into adult NSG-S mice. Engrafted BC CML or MF mouse models were randomized into (1) vehicle control, (2) mismatch control, and (3) imetelstat treatment groups (n≥3/group). Bone marrow (BM) and spleen were collected when completing the dosing plan, and the single-cell suspension was analyzed by FACS.

FIG. 4B. Bone marrow from each treatment group was collected for histological examination and FACS analysis. Left: Photomicrogaphs depict reticulin staining, DAPI staining, human CD45⁺ expression and merged DAPI and CD45 images in bone marrow collected from no transplant, vehicle, and mismatch controls as well as from imetelstat treated myelofibrosis (MF) mouse models. Right: FACS analysis plots depicting forward scatter (FSC, y-axis) and human CD45 engraftment (x-axis) in no transplant, vehicle, and mismatch controls as well as imetelstat treated MF mouse models.

FIG. 4C. FACS analysis of percentage of human CD45⁺ cell engraftment (percent) in MF mouse BM after treatment with vehicle (red), mismatch (blue) or imetelstat (green).

FIG. 4D. FACS analysis of percentage of human CD34⁺CD38⁺Lin⁻ progenitor engraftment in mouse bone marrow following MF patient (MF318) sample transplantion and treatment with vehicle (red), mismatch (blue) or imetelstat (green).

FIG. 4E. FACS analysis of percent human CD34⁺ cell engraftment in mice transplanted with pCDH lentiviral backbone (white circles; n=5) or ADAR1 overexpression (ADAR1-OE; red circles; n=6) vector transduced MF progenitor.

FIG. 4F. FACS analysis of percentage of live human CD45⁺ cells in BC CML BM engrafted mice following treatment with vehicle (red), mismatch (blue) or imetelstat (green).

FIG. 4G. FACS analysis of percentage of live human CD34⁺CD38⁺Lin⁻ progenitor cells in BC CML engrafted mice following treatment with vehicle (red), mismatch (blue) or imetelstat (green).

FIG. 4H. Self-renewal capacity of human progenitor cells was evaluated in serially transplanted mouse models. FACS analysis of percentage of live human CD34⁺CD38⁺Lin⁻ progenitor cells following serial engraftment of cells derived from mice treated with vehicle (red), mismatch (blue) or imetelstat (green).

FIG. 4I. Kaplan-Meier survival curves of mice transplanted with CD34⁺ cells selected from imetelstat treated mouse BM (green) compared with mismatch controls (blue).

FIG. 5 Telomerase Inhibition Prevents ADAR1-Activated LSC Propagation

FIG. 5A. A boxplot depicting AIMP2 Log 2 (cpm) expression by RNA-seq analysis in stem cells (left panel) and progenitors (right panel) from aged bone marrow (ABM), young bone marrow (YBM), PV, ET, MF, CML, sAML and de novo AML (dnAML).

FIG. 5B. A boxplot depicting TRF2IP Log 2 (cpm) expression by RNA-seq analysis in stem cells (left panel) and progenitors (right panel) from aged bone marrow (ABM), young bone marrow (YBM), PV, ET, MF, CML, sAML and de novo AML (dnAML).

FIG. 5C. A boxplot depicting TERT Log 2 (cpm) expression by RNA-seq analysis in human CD45⁺ cells engrafted in vehicle (red), mismatch control (blue) and imetelstat (green) treated BC CML mice.

FIG. 5D. Telomerase activity in BC CML engrafted human CD45⁺ cells, as measured by TRAP assay, in vehicle (red), mismatch control (blue) and imetelstat (green) treated BC CML mice.

FIG. 5E. β-catenin activity in human progenitor cells in BC CML mouse BM was measured by flow cytometry in vehicle (red), mismatch control (blue) and imetelstat (green) treated mice.

FIG. 5F. Boxplot depicting Log 2 (cpm) expression of ABL1 by RNA-seq analysis of human CD45⁺ cells isolated from vehicle (red), mismatch control (blue) and imetelstat (green) treated BC CML mouse cells (Student's t-test: vehicle: p=0.037, imetelstat: p=0.02).

FIG. 5G. Boxplot depicting Log 2 (cpm) expression of ADAR1p150 by RNA-seq of human CD45⁺ cells isolated from vehicle (red), mismatch control (blue) and imetelstat (green) treated BC CML mouse cells. The p-values from Student's t-tests are shown (Student's t-test: vehicle: p=0.337, imetelstat tx: p=0.016).

FIG. 5H. A boxplot depicting detectable RNA edits per million aligned reads in human CD45⁺ cells derived from vehicle (red), mismatch control (blue) and imetelstat (green) treated BC CML mouse models. The p-values from Student's t-tests are shown (vehicle: p=0.540, imetelstat tx: p=0.037).

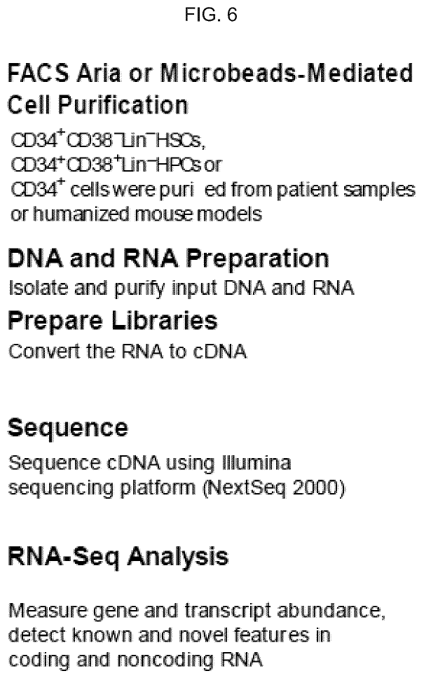

FIG. 6 schematically illustrates an exemplary S1 RNA sequencing schema (supplementary FIG. 1).

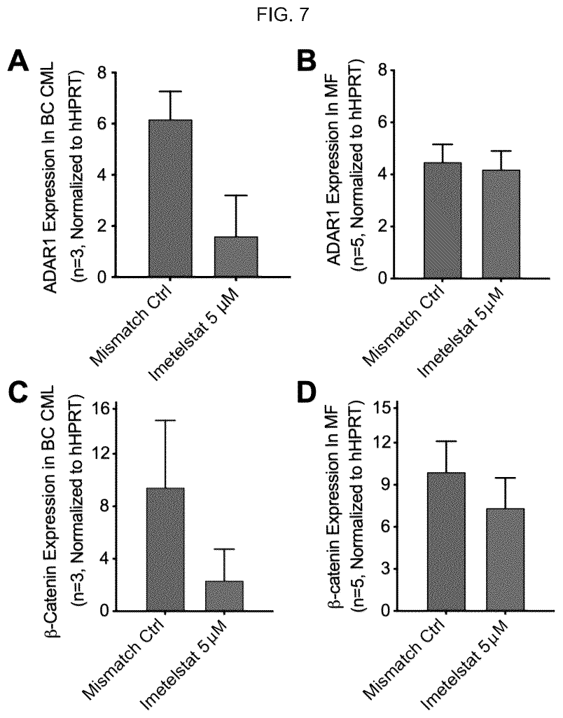

FIG. 7 illustrates supplementary FIG. 2, where gene expression qRT-PCR analyses of human CD34⁺ cells sorted from BC CML patient samples (n=3) and MF patient samples (n=5) that were co-cultured with SL/M2 stromal cells and treated with mismatch control or imetelstat at 5 μM for 14 days.

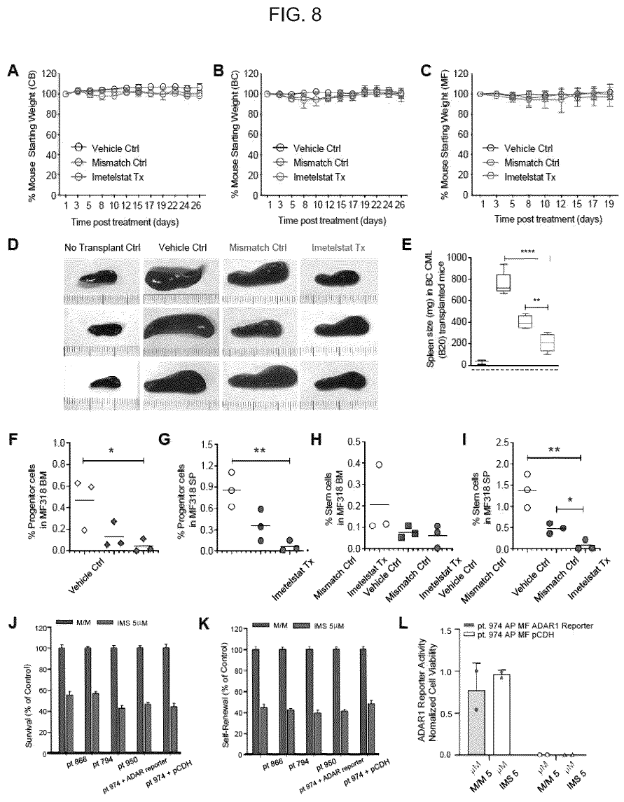

FIG. 8 illustrates supplementary FIG. 3

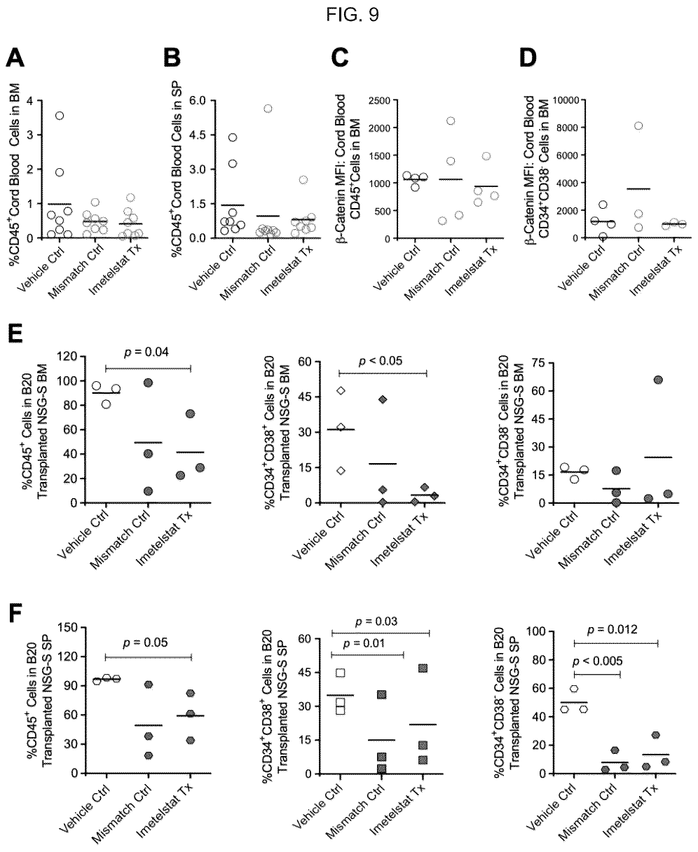

FIG. 9 illustrates supplementary FIG. 4

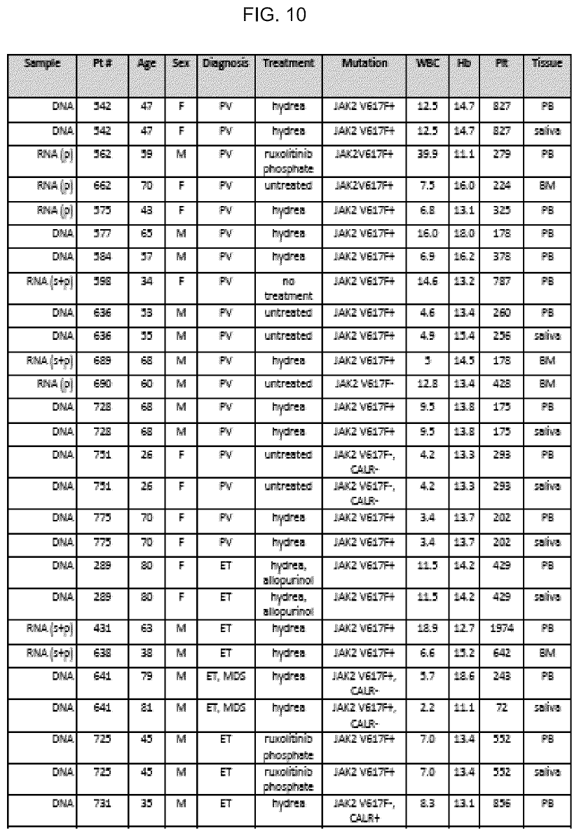

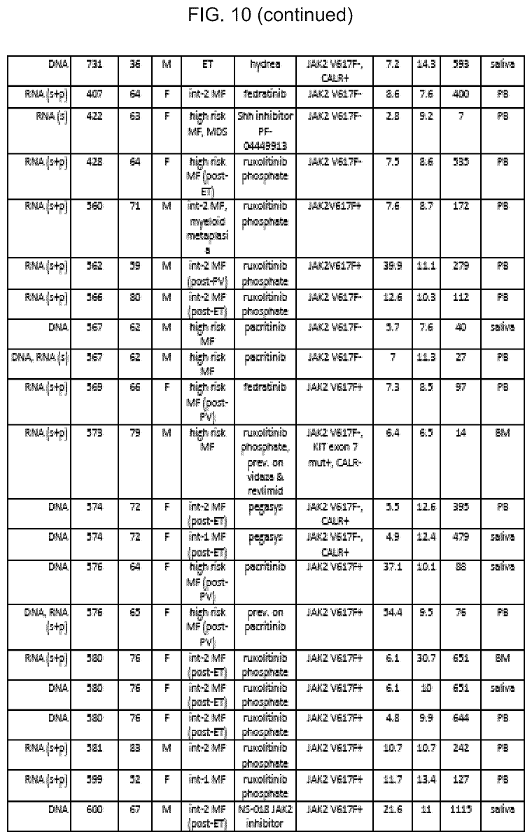

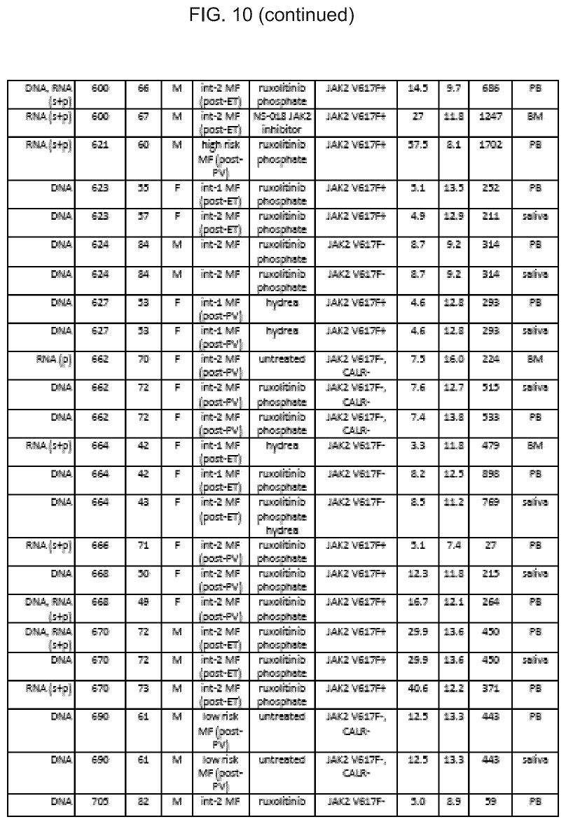

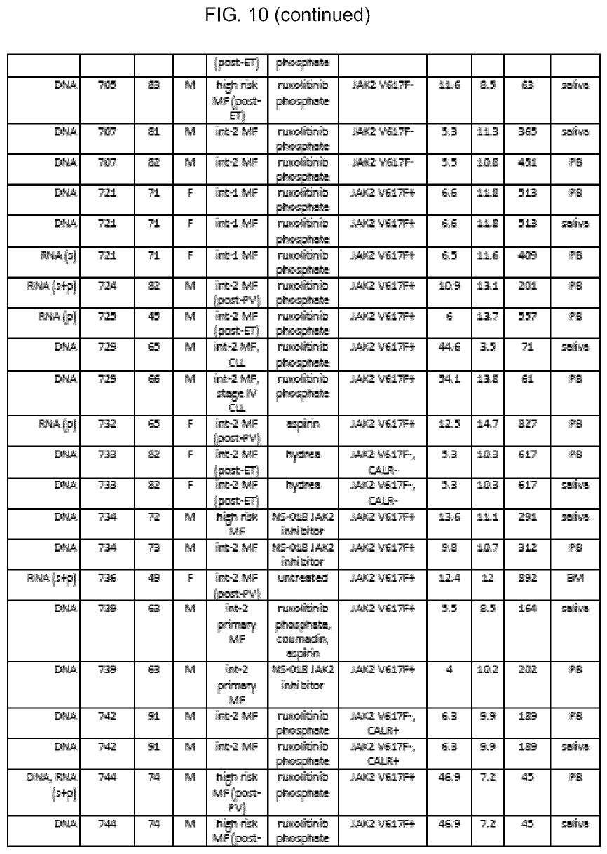

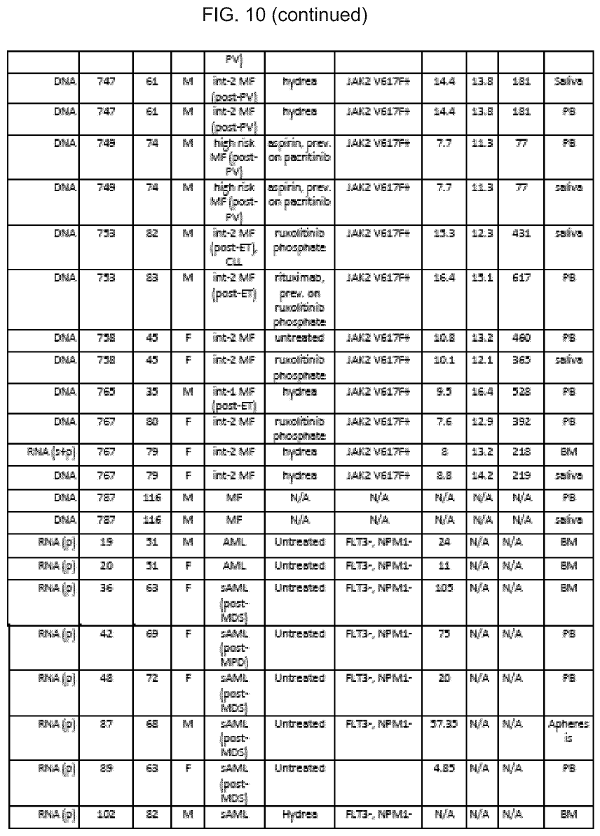

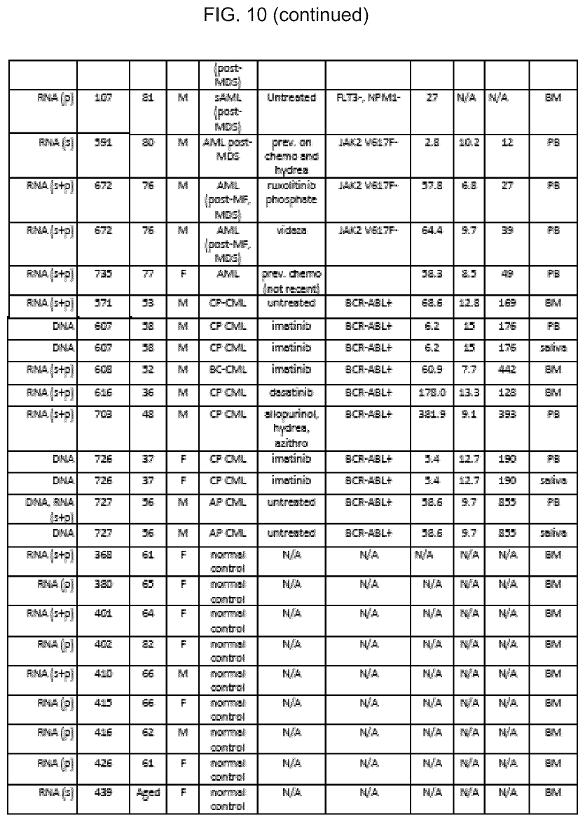

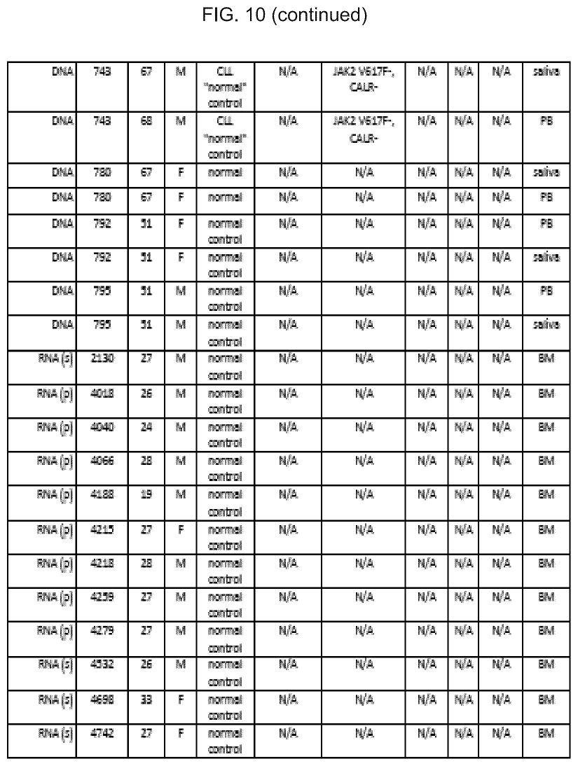

FIG. 10 illustrates supplementary Table S1.

FIG. 11 illustrates Table S2.

### Example 2: Inflammation-Driven Deaminase Deregulation Fuels Human Pre-Leukemia Cell Evolution

Inflammation-dependent base deaminases promote therapeutic resistance in many malignancies. However, their roles in human pre-leukemia stem cell (pre-LSC) evolution to acute myeloid leukemia stem cells (LSCs) had not been elucidated. Comparative whole-genome and whole-transcriptome sequencing analyses of FACS-purified pre-LSCs from myeloproliferative neoplasm (MPN) patients reveal APOBEC3C upregulation, an increased C-to-T mutational burden, and hematopoietic stem and progenitor cell (HSPC) proliferation during progression, which can be recapitulated by lentiviral APOBEC3C overexpression. In pre-LSCs, inflammatory splice isoform overexpression coincides with APOBEC3C upregulation and ADAR1p150-induced A-to-I RNA hyper-editing. Pre-LSC evolution to LSCs is marked by STAT3 editing, STAT3β isoform switching, elevated phospho-STAT3, and increased ADAR1p150 expression, which can be prevented by JAK2/STAT3 inhibition with ruxolitinib or fedratinib or lentiviral ADAR1 shRNA knockdown. Conversely, lentiviral ADAR1p150 expression enhances pre-LSC replating and STAT3 splice isoform switching. Thus, pre-LSC evolution to LSCs is fueled by primate-specific APOBEC3C-induced pre-LSC proliferation and ADAR1-mediated splicing deregulation.

Pro-inflammatory cytokine-responsive APOBEC3 (apolipoprotein B mRNA editing enzyme, catalytic polypeptide like type 3), and ADAR1 (adenosine deaminase acting on RNA 1) base deaminases restrict viral replication (Di Giorgio et al., 2020) and LINE element retrotransposition (Mannion et al., 2014; Tan et al., 2017). However, base deaminase deregulation has been linked to both genomic and epitranscriptomic (post-transcriptional modification) instability (Alexandrov et al., 2013a; Burns et al., 2013b; Han et al., 2015; Jiang et al., 2017, 2019; Lazzari et al., 2017; Peng et al., 2018; Petljak et al., 2019; Zhang and Slack, 2016; Zhou et al., 2019). In primates, APOBEC3 genes (APOBEC3A, APOBEC3B, APOBE3C, APOBEC3D, APOBEC3F, APOBEC3G, and APOBEC3H) contribute to maintenance of genomic integrity. Conversely, deregulation of APOBEC3 induces genomic instability and distinctive DNA mutational spectra in many malignancies (Alexandrov et al., 2020, 2013a, 2013b; Burns et al., 2013a) by deaminating cytidines to thymidines (C-to-T) (Buisson et al., 2019). Although APOBEC enzymes have been fused to Cas9 nuclease-defective variants to induce targeted C-to-T transitions as a stem cell gene-therapy strategy (Zafra et al., 2018), recent data suggest that APOBEC3 deaminases drive cancer-related hotspot mutagenesis (Alexandrov et al., 2020; Buisson et al., 2019). Because primate-specific APOBEC3 deaminases are activated by pro-inflammatory cytokines, such as interferon (IFN)-α and β, tumor necrosis factor (TNF)-α, and interleukin (IL)-1β and IL-6, the effects of enzymatic C-to-T deamination on the genomic landscape of cancer are inherently episodic, microenvironmentally dependent, and difficult to model (Petljak et al., 2019).

Similarly, pro-inflammatory cytokines activate ADAR1p150-mediated adenosine to inosine (A-to-I) deamination of double-stranded RNA (dsRNA), particularly in the context of primate-specific Alu sequences (Chua et al., 2020). As a dynamic regulator of mRNA and miRNA stability (Jiang et al., 2013; Lazzari et al., 2017; Tan et al., 2017), ADAR1 plays a pivotal role in embryonic development and stem cell maintenance as evidenced by murine embryonic lethality and reduced hematopoietic stem cell (HSC) multi-lineage reconstitution potential following ADAR1 deletion (Hartner et al., 2009; Jiang et al., 2017, 2019; Zipeto et al., 2016). Moreover, a recent study showed that ADAR1 loss reduces induced pluripotent stem cell (iPSC) reprogramming efficiency by inducing ER stress (Guallar et al., 2020). Additionally, alternatively spliced regions frequently harbor A-to-I editing sites whereby ADAR1 deficiency impairs alternative splicing in mouse tissues (Kapoor et al., 2020). Deregulation of ADAR1-mediated A-to-I editing alters stem cell survival and self-renewal regulatory mRNA and miRNA stability (Chen et al., 2013; Han et al., 2015; Jiang et al., 2017, 2019; Lazzari et al., 2017; Zipeto et al., 2016). Although deaminase deregulation has been linked to therapeutic resistance in many tumor types (Han et al., 2015; Lazzari et al., 2017), the combinatorial capacity of APOBEC3-induced DNA mutagenesis and ADAR1-mediated splicing disruption and epitranscriptomic instability to fuel pre-LSC transformation into LSCs has not been elucidated.

As an important paradigm for understanding molecular drivers of progression to secondary acute myeloid leukemia (sAML), myeloproliferative neoplasms (MPNs), including polycythemia vera (PV), essential thrombocythemia (ET), myelofibrosis (MF), and chronic myeloid leukemia (CML), were the first malignancies shown to harbor somatic stem cell mutations (Eide and Druker, 2017; Jiang et al., 2017; Rossi et al., 2008). Recent reports suggest that AML transformation is not solely predicted by the baseline driver mutations (i.e., JAK2-V617F, CALR, MPL) or additional somatic mutations (i.e., ASXL1, EZH2) (Tefferi et al., 2018) but has been associated with leukocytosis, constitutional symptoms, and pathologically increased cytokines, such as IL-8 (Tefferi et al., 2011). Increases in pathologically induced cytokines have been found in MPNs (Pardanani et al., 2013) and decreases in these cytokines by JAK inhibition (Verstovsek et al., 2012) may well be responsible for observed improvements in survival and decreases in risk of leukemic transformation (Verstovsek et al., 2017).

Pre-leukemia stem cells (pre-LSCs) in MPNs arise from clonally mutated hematopoietic stem and progenitor cells (HSPCs) that vary in their capacity to become dormant, resist therapy (Gishizky et al., 1993; Jamieson et al., 2004; Kleppe et al., 2018; Rossi et al., 2008), and contribute to the generation of LSCs that drive sAML transformation (Mesa et al., 2017; Shlush et al., 2014). While the propensity of MPNs to transform to sAML has been difficult to ascertain based on standard prognostic guidelines (Mesa et al., 2017; Shlush et al., 2014), seminal studies demonstrate that MPN initiation is driven by heritable risks and that MPNs harbor distinctive mutational profiles that predict progression as well as overall survival (Bao et al., 2020; Grinfeld et al., 2018; Miles et al., 2020). Moreover, in response to microbial signals, IL-6 production has been shown to promote pre-leukemic myeloproliferation in Tetmethylcytosine dioxygenase 2 (Tet-2)-deficient mice (Meisel et al., 2018) thereby underscoring the importance of episodic, pro-inflammatory cytokine induction of MPN progression. However, the primate-specific impact of cytokine-induced enzymatic mutagenesis had not been addressed. Thus, we investigated the combinatorial capacity of inflammation-dependent, primate-specific APOBEC3 and ADAR1 deaminases to drive human pre-LSC evolution to LSCs with the ultimate aim of informing the development of effective strategies that predict and prevent transformation to rapidly fatal sAML.

### Results

APOBEC3C deaminase activation promotes human Pre-LSC proliferation To identify pre-LSC DNA mutational hierarchies and deaminase mutational signatures, whole-genome sequencing (WGS) analysis was performed on CD34⁺ stem cells purified from peripheral blood of 39 individuals with various MPNs, as well as 4 non-MPN controls, including 1 chronic lymphocytic leukemia (CLL) (FIG. 1A; Table S1). Somatic mutations were identified in the genomes of CD34⁺ stem cells from the 39 MPN patients using two complementary approaches: (1) ensemble variant calling comparing CD34⁺ stems cells in peripheral blood to bulk saliva; and (2) identification of somatic mutations, without using matched normal tissues, by employing tumor-only somatic variant filtering. These two complementary approaches were used to mitigate the risk of identifying somatic mutations in the setting of matched-normal tissue (i.e., saliva) contamination with MPN cells (i.e., peripheral blood).

Using this combined approach, we compared the somatic mutations derived using ensemble variant calling in our CD34⁺ MPN stems cells (MCCWG CD34⁺ MPNs) to the ones derived using ensemble variant calling by the PCAWG consortium in MPN bulk peripheral blood. Remarkably, the tumor mutational burden was lower in stem cells compared to bulk blood with a more than 4-fold depletion of single point mutations observed in CD34⁺ MPN cells (p<0.0012; FIG. 1B). Furthermore, focused interrogation of 69 MPN-associated genes (Grinfeld et al., 2018) provided additional confirmation of a low mutational burden in MPN stem cells compared to previously published datasets (FIGS. 1B and 1C). Overall, these results suggest that more slowly cycling pre-leukemic MPN stem cells MPNs, as well as 4 non-MPN controls, including 1 chronic lymphocytic leukemia (CLL) (FIG. 1A; Table S1). Somatic mutations were identified in the genomes of CD34⁺ stem cells from the 39 MPN patients using two complementary approaches: (1) ensemble variant calling comparing CD34⁺ stems cells in peripheral blood to bulk saliva; and (2) identification of somatic mutations, without using matched normal tissues, by employing tumor-only somatic variant filtering. These two complementary approaches were used to mitigate the risk of identifying somatic mutations in the setting of matched-normal tissue (i.e., saliva) contamination with MPN cells (i.e., peripheral blood).

Using this combined approach, we compared the somatic mutations derived using ensemble variant calling in our CD34⁺MPN stems cells (MCCWG CD34⁺ MPNs) to the ones derived using ensemble variant calling by the PCAWG consortium in MPN bulk peripheral blood. Remarkably, the tumor mutational burden was lower in stem cells compared to bulk blood with a more than 4-fold depletion of single point mutations observed in CD34⁺MPN cells (p<0.0012; FIG. 1B). Furthermore, focused interrogation of 69 MPN-associated genes (Grinfeld et al., 2018) provided additional confirmation of a low mutational burden in MPN stem cells compared to previously published datasets (FIGS. 1B and 1C). Overall, these results suggest that more slowly cycling pre-leukemic MPN stem cells may be less mutable than the more highly proliferative bulk blood cells. Further analysis confirmed that clock-like mutational signatures perfectly recapitulate the patterns observed in both PCAWG MPNs derived from bulk blood and MCCWG MPNs derived from CD34⁺ stem cells (cosine similarities: 0.97 and 0.94, respectively).

Interestingly, COSMIC signature SBS1, a clock-like signature associated with cell division, was depleted in pre-leukemic MPN stem cells compared to proliferative bulk blood, while COSMIC signature SBS5, a clock-like signature putatively associated with circadian rhythm, was highly prominent in MPN CD34⁺ stem cells (FIG. 1B).

Subsequently, single-nucleotide variant, copy-number variant, and structural variant analyses were performed on all samples, employing tumor-only somatic variant filtering on the peripheral blood samples and subtracting structural and copy-number variants found in the non-MPN samples as common germline variants (Figure S1A). Mutations were found in inflammation-related genes, such as ILIA (SNP) and IRF1 (duplication in 1 PV and 4 MF patients) (Figure S1A), a known driver of APOBEC3 activation. Except for patients presenting with high-risk MF, the genes JAK2, CALR, ASXL1/3, and KAIT2C were mutated in MPN CD34⁺ stem cell populations (FIG. 1C) at a lower frequency than bulk peripheral blood samples (FIG. 1C). Some high-risk MF patients with mutations in MPN-associated genes harbored another malignancy or progressed to AML following sample collection indicative of genomic and/or transcriptomic instability, as has been described for malignancies with APOBEC3-related mutational signatures and ADAR1 activation (FIG. 1C; Figures S1B-S1F; Table S1).

The most commonly observed mutations in MPN CD34⁺ cells were C-to-T transitions (approximately 50%) followed by T-to-C changes (approximately 20%) (FIGS. 1C and 1D). Frequent C-to-T mutations have been reported following activation of APOBEC3 (Alexandrov et al., 2013a; Burns et al., 2013b). Therefore, we examined the expression of APOBEC3 and other transcripts in MPN stem and progenitor populations by RNA sequencing (RNA-seq) (FIGS. 1E and 52A-52E). Notably, APOBEC3C was upregulated in national comprehensive cancer network (NCCN) panel guideline-defined intermediate-risk (Int-MF) and high-risk myelofibrosis (HR-MF) stem cell-enriched samples, suggesting a role for APOBEC3C in pre-LSC propagation (FIG. 1E; Figure S2E). Consistent with this hypothesis, we observed proliferation of CD34⁺ hematopoietic stem and progenitor cells (HSPCs) following lentiviral APOBEC3C overexpression as well as expansion of the stem cell population following MF progression to AML (FIG. 1F). Following lentivirally enforced APOBEC3C expression in CD34⁺ cord-blood stem cells, WGS revealed a pattern of mutations similar to that of MPN CD34⁺ cells (cosine similarity: 0.96; FIG. 1B and Figure S1B) suggesting that APOBEC3C contributes to MPN stem cell mutagenesis. In contrast to an editase-deficient mutant APOBEC3C, lentiviral APOBEC3C wild-type overexpression in CD34⁺ cord-blood cells resulted in expansion of a progenitor population that lacks replating capacity and skews toward the erythroid lineage as evidenced by increased erythroid colony formation (FIGS. 1G and 1H; Figures S1C and S1F). Lentiviral overexpression of another inflammation-responsive deaminase, ADAR1, induced expansion of a CD19⁺ B cell population (Figure S1E). These results indicate that APOBEC3C and ADAR1 play regulatory roles in hematopoietic cell-fate determination. While APOBEC3 deaminases are drivers of somatic mutagenesis in many human cancers (Burns et al., 2013b), this report provides a mechanistic link between APOBEC3C and pre-LSC generation and identifies the differential roles of APOBEC3C and ADAR1 in human HSPC expansion and cell-fate determination.

### Inflammatory Pathway Activation in Pre-LSCs

To comprehensively investigate inflammation-dependent deaminase induction of pre-LSC evolution, a comparative WGS and RNA-seq analysis pipeline (FIG. 1A; Figure S2A-S2E) was established to exclude naturally occurring single-nucleotide polymorphisms (SNPs) and enable detection of previously unreported editing sites in the setting of deaminase activation. To detect inflammatory pathway activation during MPN progression, RNA-seq analyses were performed on 113 fluorescence-activated cell sorting (FACS)-purified stem cell (CD34⁺CD38⁻Lin) and progenitor (CD34⁺CD38⁺Lin⁻) populations from 54 MPN and AML patients and 24 young and aged healthy controls (FIG. 1A; Figure S2A; Figure S2B). A gene-expression signature emerged that distinguished MPN and AML progenitors from stem cells in normal young and aged samples (Figure S2B). A comparison between AML and MF samples elucidated 987 and 678 differentially expressed genes in stem cells and progenitors, respectively (Figure S2D). Notably, transcripts involved in regulation of inflammation, including CTSA (cathepsin A) and inflammatory cytokine receptor genes (CD97 and EFHD2), were increased in AML stem cells and progenitors relative to MF, suggesting that deregulated inflammatory pathways may contribute to pre-LSC transformation into LSCs (Figure S2D; Figures S3A-S3H).

MPN stem cells harbored only 24 common differentially expressed genes relative to healthy aged bone marrow (ABM) (Figure S3A). Notably, interferon-stimulated gene (ISG) activators of ADAR1, such as IRF9 and IFI77111, were overexpressed in PV stem cells (Figure S3B). In MF progenitors, expression of CSNK1γ2, a WNT-β-catenin self-renewal pathway regulator, was elevated relative to ABM (Figures S3D and S3H). In AML stem and progenitor cells, both IER2 and CSF1R were upregulated, which have been associated with increased cytokine responsiveness as well as release of pro-inflammatory chemokines promoting invasion and metastasis (Figures S3B, S3D, S3G, and S3H). Last, comparative RNA-seq analyses revealed that the top enriched signaling pathways in sAML compared with MF were related to inflammation, autoimmunity, and WNT signaling further underscoring the importance of inflammatory cytokine signaling and WNT/β-catenin self-renewal pathway activation in pre-LSC evolution to LSCs (Figures S3B-S3F).

Inflammation-dependent ADARp150 and APOBEC3C promote pre-LSC evolution The predominance of inflammatory signatures detected in pre-LSCs led us to examine the combined roles of inflammatory cytokine-inducible ADAR1 and APOBEC3 deaminases in MPN progression to AML. Initial hierarchical clustering of RNA-seq analyses, including the top 1% of genes ranked by variance across the dataset, revealed distinct gene (Figure S2B) and splice isoform expression patterns between normal, MPN, and AML hematopoietic stem cell (HSC) and progenitor samples (FIG. 2A). A predominance of IFN- or inflammatory cytokine-related transcripts, including a pro-inflammatory IL6ST isoform, was observed in MPN and sAML progenitors relative to ABM controls (FIG. 2B). Moreover, MPN and AML progenitors displayed a splice isoform switch favoring expression of the inflammatory cytokine-responsive ADAR1 p150 isoform over the constitutively active ADAR1 p110 isoform (FIG. 2C).

Because of the observed upregulation of inflammatory cytokine-related genes capable of activating innate immune deaminases in MPNs, we interrogated the differentially expressed genes by performing signaling pathway impact analysis (SPIA) comparing normal ABM with MPN and AML progenitors (FIG. 2D). Comparison of MPN with ABM revealed activation of pathways involved in regulation of chemokine signaling, RNA transport, and transcriptional deregulation in cancer (FIG. 2D). Notably, approximately 70% of the genes in the influenza A pathway were differentially expressed in ET, PV, and AML when compared to normal samples (FIG. 2D). Moreover, both Epstein-Barr virus (EBV) and Influenza A viral infection-related pathways were activated in AML compared with ABM indicating that anti-viral pathway activation is associated with LSC generation (FIG. 2D). To investigate whether ADAR1 contributed to pre-LSC transformation (Jiang et al., 2017), we performed RNA-seq following lentiviral transduction of cord-blood stem or progenitor cells with ADAR1 wild type (ADAR1 WT) or a deaminase-inactive mutant (ADAR1^{E912A}) (Jiang et al., 2019). SPIA analysis revealed that two KEGG pathways activated by ADAR1 WT overexpression in both cell types were involved in cancer and viral carcinogenesis (FIG. 2E) thereby mirroring the viral pathway activation signature typical of ADAR1-overexpressing AML progenitors (FIG. 2D). Together, these data suggest that inflammatory cytokine-inducible ADAR1p150 expression contributes to pre-LSC maintenance and LSC generation.

While activation of ADAR1 has been linked to cancer progression (Jiang et al., 2013, 2017; Peng et al., 2018), the combinatorial role of APOBEC3C and ADAR1p150 in pre-LSC evolution to LSCs had not been examined. Both ADAR1p150 and APOBEC3C transcripts were elevated by RNA-seq analyses in high-risk MPN stem cells (FIG. 2 F; Table S1). Co-immunoprecipitation analysis revealed that APOBEC3C and ADAR1p150 bind to each other in HEK293T cells (FIG. 2G). Moreover, confocal fluorescence microscopy revealed that co-localization of ADAR1p150 and APOBEC3C in AML cells was reduced by robust lentiviral ADAR1 short hairpin RNA (shRNA) knockdown (FIG. 2H; Figure S2C). These data suggest that activation of both APOBEC3C and ADAR1p150 fuels pre-LSC evolution.

A-to-I RNA Editing Signatures Distinguish Pre-LSCs from LSCs

To further evaluate the impact of ADAR1 activation on pre-LSC evolution to LSCs, we analyzed epitranscriptomic alterations observed in our samples that are present in the REDIportal atlas of A-to-I RNA editing that contains over 16 million events curated from RNA-seq analyses derived from 549 individuals. We identified known and previously unknown RNA editing events in MPN and AML (secondary and de novo) as well as normal young bone marrow (YBM) and ABM samples. Each MPN subtype and untreated sAML possessed significantly elevated levels of RNA editing compared to ABM controls as measured by the median variant allele frequency (VAF) (FIG. 3A). Inflammatory cytokine-inducible ADAR1p150 expression positively correlated with editing activity in MPN and sAML progenitors suggesting that inflammatory cytokine signaling promotes malignant editing in pre-LSCs and LSCs (FIG. 3B; FIG. S3A-S3H; Figure S4A). To determine the frequency of RNA editing in different transcriptomic regions, we analyzed the VAF associated with A-to-I editing changes in ABM, YBM, MPN, and AML progenitors (FIG. 3C). Compared with ABM and YBM, MPN progenitors harbored higher levels of A-to-I editing that resulted in non-synonymous changes (FIG. 3D). Notably, 3′ UTR editing increased in MF and persisted in untreated sAML (FIG. 3D). Thus, ADAR1p150 induces A-to-I editing events that may prevent transcript targeting by microRNAs in 3′ UTR regions and thereby enhance transcript stability in MF and sAML (Jiang et al., 2019).

Current RNA editing databases are primarily based on cell-line or bulk tumor cell RNA-seq data that may mask the cell-type and context-specific RNA editing events that trigger pre-LSC evolution into LSCs. To identify RNA editing sites specific to pre-LSCs, we compared RNA-seq variants with matching WGS data (FIGS. 1A-1C) and quantified non-synonymous editing events using REDIportal and two other established RNA editing databases (Kiran and Baranov, 2010; Ramaswami and Li, 2014) (FIG. 3E). We observed a strikingly different editing pattern between MPN stem cells and progenitors and ABM and YBM control samples. While missense editing of CDK13, a splicing and cell-cycle regulatory gene, occurred in 84% of MPN samples, missense editing was not detected in ABM and YBM (FIG. 3E). Moreover, CDK13 transcript levels were elevated in MF (p<0.0001) and sAML stem cells (p<0.0001) and progenitors (FIG. 3F). Additionally, increased CDK13 transcript levels correlated with ADAR1 overexpression (FIG. 3G). Furthermore, confocal fluorescence microscopy revealed CDK13 upregulation and increased ADAR1 in myeloid leukemia cells consistent with CDK13 transcript stabilization and subsequent increased translation as a result of ADAR1 upregulation (FIG. 3H). While A-to-I RNA editing and stabilization of CDK13 transcripts have been linked to a worse prognosis in hepatocellular carcinoma (Dong et al., 2018), CDK13 targeting with a covalent inhibitor, THZ531, has potent anti-tumor activity suggesting that this approach may have further utility in LSC targeting (Iniguez et al., 2018). Conversely, a SUMF2 missense (recoding) editing was more prevalent in normal controls than MPN samples (FIG. 3E; Figures S4B and S4H-S4J). While previous studies indicate that ADAR1-mediated A-to-I editing events occur predominantly in Alu repetitive element containing dsRNA structures (Jiang et al., 2017), our MPN stem and progenitor cell-enriched RNA-seq analysis of the nonsynonymous editing events reveals that recoding events also occur in non-A/u regions (FIG. 3E). Together these data suggest that distinctive RNA editing events predict MPN initiation and progression.

Subsequently, we examined the expression of all 1,295 differentially edited genes in MPN, AML, and ABM stem and progenitor cells. Hierarchical clustering of the gene-expression values for differentially edited genes revealed that MPN samples clustered together when compared with normal young and aged controls (FIG. 4A). Notably, AML samples tended to cluster closer to normal samples indicative of reversion to stemness typical of aggressive malignancies. Further analysis of the differentially edited genes propagated over the STRING (Szklarczyk et al., 2017) interactome and clustered with Louvain clustering revealed that in both AML and MF progenitors A-to-I RNA edited transcripts were significantly enriched for genes involved in chromatin organization, transcription, and mRNA splicing (FIGS. 4 B and 4C). Interestingly, AML progenitors demonstrated differential editing of ribosomal regulatory genes compared to ABM thereby suggesting that disruption of translational control by ADAR1p150 activation may fuel pre-LSC transformation.

To further investigate the role of ADAR1 in clonal evolution, we performed single-cell RNA-seq (scRNA-seq) of CD34⁺ cord-blood cells transduced with a lentiviral backbone control (shCTRL) or lentiviral shRNA targeting ADAR1 (shADAR1) (Figure S4C; Table S2). In keeping with a ribosomal regulatory role for ADAR1, tSNE analysis revealed differential ribosomal gene editing following ADAR1 knockdown (Figure S4D) (Solomon et al., 2017). Notably, ribosomal transcripts were also differentially edited in MF and AML compared with normal age-matched progenitors (FIGS. 4B and 4C; Figures S4E-S4G). These editing-induced changes in ribosomal gene expression suggest that ADAR1 activation may alter protein turnover rates in LSCs. These observations correspond with previous reports showing disruption of proteostasis (i.e., protein turnover) as a driver of LSC propagation in mouse models of leukemia (Signer et al., 2014) and ADAR1-induced proteomic diversity as a contributor to therapeutic resistance in a broad array of malignancies (Chua et al., 2020; Peng et al., 2018).

### RNA-Editing-Induced STAT3 Splice Isoform Switching Induces Pre-LSC Evolution to LSCs

While the overall A-to-I RNA editing events increased in MPN progenitors compared with their normal counterparts, progression of MF and sAML was marked by increased editing of specific regions, including in lincRNA and in 3′ UTRs (FIG. 3D), as well as of selective transcripts (FIGS. 4A-4C). Notably, STRING interactome analysis revealed that a transcriptional activator of ADAR1 and embryonic self-renewal agonist, STAT3, was differentially edited and overexpressed in AML compared to normal progenitors (FIG. 4C). Previously, we reported that the JAK/STAT signaling pathway activates malignant A-to-I RNA editing in stem cell regulatory transcripts and increases LSC self-renewal capacity in CML (Zipeto et al., 2016). Since A-to-I RNA editing can remove the 3′ splice acceptor adenosine, we investigated whether ADAR1 activity is linked to pre-mRNA splicing changes and expression of alternative STAT3 isoforms.

Alternative splicing in STAT3 exon 23 generates two isoforms, STAT3α and STAT3β (FIG. 5A; Figures S5A-S5H). A previous report showed that intronic RNA editing of STAT3 favored increased expression of the relatively rare STAT30 splice isoform (Goldberg et al., 2017). In AML, 4 out of 7 samples harbored A-to-I RNA editing at previously validated STAT3 intronic editing sites, whereas only one ABM sample possessed these STAT3 RNA editing events (FIG. 5B). The expression of alternatively spliced STAT3/3 increased in MPN and AML stem cells and progenitors with elevated ADAR1p150 expression compared to ABM controls (FIG. 5C; Figures S5A and S5B). Following exposure to inflammatory cytokines, western blot analyses revealed that ADAR1p150 levels were elevated in sAML (pt.255 and pt.705) CD34⁺ cells compared to MF (pt. 749) or normal CD34⁺ cord-blood cells (FIG. 5D). Notably, expression of the ADAR1 RNA editing-induced phospho-STAT3β isoform increased in sAML compared to normal controls (FIG. 5B; Figures S5A and S5B). In keeping with ADAR1 induction of alternative STAT3 splice isoform usage, lentiviral ADAR1 overexpression in primary MPN progenitors was associated with increased STAT3 splice isoform expression (FIG. 5F; FIG. S5H). Conversely, treatment with selective JAK2 inhibitors, ruxolitinib or fedratinib, in the presence of inflammatory cytokines, reduced the levels of ADAR1p150 and phospho-STAT3 in sAML stem cells (FIG. 5E; Figure S5F). Following IFN-alpha treatment of myeloid leukemia cells, lentiviral ADAR1 shRNA knockdown reduced phosho-STAT3β expression (Figures S5E and S5G). Thus, STAT3 editing increases overall phospho-STAT3, which can bind to the ADAR1 promoter and activate ADAR1 transcription. This feedback loop contributes to LSC generation and can be disrupted by pharmacologic JAK2/STAT3 inhibition or ADAR1 shRNA knockdown.

As a self-renewal agonist, STAT3 represses GSK3β via ARID1 and prevents phosphorylation as well as subsequent degradation of β-catenin (Bowman et al., 2001; Hirai et al., 2011; Nusse and Clevers, 2017; Wu et al., 2016). In keeping with the LSC-propagating effects of STAT3 upregulation, engrafted sAML progenitors expressed phospho-STAT3 as measured by flow cytometric evaluation of humanized sAML mouse model bone marrow (FIG. 5G). Moreover, lentivirally enforced ADAR1 expression in pre-LSCs increased colony replating to a level more typical of LSCs in sAML (FIG. 5H; Figures S5C and S5D). Together, these data suggest that ADAR1 A-to-I editing-induced STAT3 splice isoform switching promotes transformation of pre-LSCs into LSCs that drive therapy-resistant sAML transformation.

### Discussion

In addition to environmental mutagens, cumulative data suggest that APOBEC3 DNA and ADAR1 RNA deaminases serve as enzymatic drivers of cancer evolution (Alexandrov et al., 2013a, 2013b; Burns et al., 2013a; Chua et al., 2020; Jiang et al., 2017). Specifically, patterns of C-to-T deamination induced by aberrant activation of APOBEC3 family members have been identified by whole-exome sequencing in many human malignancies (Burns et al., 2013b). Moreover, inflammatory cytokine-induced hyperactivation of ADAR1p150 results in A-to-I deamination of self-renewal and cell-cycle regulatory transcripts thereby fueling therapeutic resistance in leukemia (Jiang et al., 2013, 2019; Lazzari et al., 2017; Zipeto et al., 2016). However, the combinatorial roles of APOBEC3 and ADAR1 in primary human pre-cancer stem cell evolution to therapy-resistant cancer stem cells had not been examined.

In this study, we focused on characterizing the combined capacity of enzymatic DNA and RNA deamination to induce pre-LSC evolution to LSCs that fuel sAML transformation. To this end, we established (1) primary human stem and progenitor cell-based WGS and RNA-seq editome analysis pipelines that enabled identification of pre-LSC and LSC-specific DNA and RNA editing sites, (2) lentiviral APOBEC3C wild-type and mutant functional impact analysis in hematopoietic stem and progenitor cells, and (3) lentiviral ADAR1 wild-type, mutant, and shRNA knockdown analysis of RNA editing-related recoding events and splicing alterations. Using these combined human hematopoietic stem and progenitor cell-focused molecular and functional analysis strategies, we discovered that APOBEC3C activation fuels C-to-T mutagenesis and expansion of MPN progenitors with increased ADAR1p150 A-to-I RNA editing capacity thereby initiating LSC generation. Confocal fluorescence microscopic analyses showing APOBEC3C and ADAR1p150 co-localization together with co-immunoprecipitation of APOBEC3C and ADAR1p150 data suggest that these enzymes may function as a complex to increase deamination of cytosolic single-stranded DNA (ssDNA) and dsRNA in response to increased systemic inflammatory cytokine signaling or in response to viral replication.

In MPN stem and progenitor cells, APOBEC3C induces proliferation and C-to-T mutagenesis that sets the stage for inflammatory cytokine-induced ADAR1p150 activation resulting in CDK13 missense editing and transcript instability, which has been linked to decreased survival of patients with therapeutically recalcitrant malignancies (Dong et al., 2018). Ultimately, inflammation-responsive ADAR1p150 activation in APOBEC3C-overexpressing MPN pre-LSCs induces STAT3 intronic editing, which increases expression of STAT3β. In a previous study, STAT3β was found to repress GSK3β and ARID1B thereby preventing phosphorylation and subsequent degradation of β-catenin (Wu et al., 2016), which is frequently deregulated in LSCs (Jamieson et al., 2004; Zipeto et al., 2016). In keeping with this report, we found that ADAR1p150 upregulation was associated with increased STAT3β splice isoform expression in LSCs. Moreover, ADAR1p150 induction could be reduced by pharmacologic JAK2/STAT3 inhibition with fedratinib or ruxolitinib and could be phenocopied by lentiviral ADAR1 shRNA knockdown. In the setting of increased APOBEC3C expression by high-risk MF pre-LSCs, lentiviral ADAR1 overexpression enhanced colony replating efficiency, as an in vitro surrogate measure of self-renewal, suggesting that APOBEC3C and ADAR1 work in concert to induce pre-LSC evolution to LSCs. If a pro-inflammatory microenvironment in the marrow is conducive to the accumulation of additional somatic mutations driving pre-LSC transformation into LSCs, this could explain observed clinical benefits of MPN therapy. Indeed, the decreases seen in disease progression in MPNs, both from PV to MF by therapy with long-acting interferons (Jager et al., 2020), as well as decreases in progression from MF to AML with JAK inhibition (Verstovsek et al., 2017; Vannucchi et al., 2017; Yacoub et al., 2019) may well originate from decreasesin an inflammatory microenvironment in the marrow.

In addition to providing a robust framework for predicting and preventing pre-cancer stem cell evolution, the discovery of combined base deaminase deregulation is particularly relevant for identifying the potentially pre-malignant consequences of clinical gene-therapy strategies (Komor et al., 2016) involving APOBEC3 and ADAR base editors. Because APOBEC3 and ADAR1 can be activated by cytosolic ssDNA, dsRNA structures, and lentiviral transduction, they may contribute to DNA mutations and RNA alterations induced by CRISPR-Cas guided base editing technologies as well as lentivirally delivered therapeutic gene-correction strategies. Also, the differential effects of APOBEC3 and ADAR1 on stem cell-fate specification will need to be considered prior to implementation of stem cell gene-therapy approaches involving base editing technologies. The potential for induction of both genomic and epitranscriptomic instability provides a strong rationale for deciphering the oncogenic potential of combinatorial APOBEC3 and ADAR1 activation (Grunewald et al., 2019).

While we focused on the C-to-T DNA mutational impact of APOBEC3C overexpression, other APOBEC3 enzymes, such as APOBEC3A, can also induce DNA editing in response to IFN thereby promoting genomic instability (Sharma et al., 2015). Moreover, the RNA editing capacity of APOBEC3C and other APOBEC3 enzymes has not been clearly elucidated in stem cells and forms the basis for launching whole-transcriptome and single stem cell RNA-seq analyses as well as functional stem cell impact studies. Moreover, C-to-T deamination by APOBEC3C could remove cytosine thereby preventing cytosine methylation. Because cytosine demethylation represents a major cancer mutational signature, the role of APOBEC3C in the induction of malignant genetic modifications that determine expression patterns for a large set of genes will need to be further studied.

In contrast to normal HSPCs, inflammatory cytokines induce both APOBEC3C and ADAR1 expression in pre-LSCs thereby promoting evolution to LSCs in sAML. Further investigation into whether sustained ADAR1 activation occurs as a result of downregulation of an ADAR repressor, like AIMP2, which enhances degradation of ADAR proteins (Tan et al., 2017), or whether ADAR1 hyperactivation promotes malignant reprogramming of pre-LSCs into LSCs by altering ER stress responses (Guallar et al., 2020), may provide additional insights into the cell-type and context-specific causes and functional consequences of deaminase deregulation.

Both ADAR1 and APOBEC3 play important roles in the intrinsic responses to viral infection and protect the human genome from retrotransposition. They also play important roles in innate and adaptive immunity by controlling the response to inflammatory cytokine signals. In keeping with the induction of deaminases by inflammatory cytokines, we found that the top activated genes in pre-LSCs compared with normal HSPC controls corresponded with anti-viral signaling pathways and chemokine signaling. The most common anti-viral signature was related to EBV infection (ET, PV, and AML), which is associated with viral oncogenesis. These data suggest that inflammation-dependent deaminases induced by viral infection, human endogenous retroviral activation, LINE element retrotransposition, or chronic cytokine signaling promote MPN pre-LSC transformation into LSCs in sAML and will need to be further studied with viral transcriptome analysis pipelines. Also, recent studies suggest that deletion of ADAR1 sensitizes malignant cells to PD-1 immune checkpoint blockade (Ishizuka et al., 2019). Thus, early detection and targeted inhibition of combined APOBEC3C and ADAR1 activation may have important implications for preventing human pre-cancer stem cell evolution to cancer stem cells that promote therapeutic resistance and disease progression.

### METHODS Example 2:

### Patient Sample Processing and Preparation for Whole-Genome Sequencing

CD34⁺ cells: Peripheral blood mononuclear cells were isolated by Ficoll-paque density centrifugation and cryopreserved in liquid nitrogen. CD34⁺ cells were selected from peripheral blood mononuclear cells from both MPN patients and normal controls by magnetic bead separation (MACS; Miltenyi, Bergisch Gladbach, Germany) as previously described (Jiang et al., 2013) with minor modification using a different kit for magnetic bead separation: Catalog 130-100-453. DNA from the peripheral blood CD34⁺ population was extracted according to manufacturer recommendations using QIAamp DNA Blood Mini Kit (QIAGEN, Catalog number 51104).

Saliva cells: Subjects abstained from eating at least 1 hour prior to saliva donation and rinsed their mouths with water to remove food residue immediately prior to saliva donation. Subjects then deposited 1 mL of saliva into the collection device, which was stabilized immediately afterward (Biomatrica, Catalog number 97021-011A). Stabilized saliva was passed through 70-100 micron strainers to further remove food residues. DNA was extracted using the QIAamp DNA Blood Mini Kit (QIAGEN, Catalog number 51104) described above with minor modifications. Both peripheral blood (90×) and saliva (30×) cell samples were sequenced on the Illumina HiSeq X sequencer using a 150-base paired-end single-index read format.

Patient Sample processing and Preparation for Whole-transcriptome Sequencing

Whole-transcriptome sequencing (RNA-seq) was performed on 78 samples distributed as follows: PV (n=6), ET (n=2), MF (n=29), CML (n=5), AML (n=12), and non-MPN control individuals (n=24). These samples can further be broken down based on tissue of collection (peripheral blood or bone marrow) and cell types (stem cells and progenitor). In summary, from 54 subjects and 24 non-MPN controls, 113 samples were included in the RNA sequencing cohort. Mononuclear cells from peripheral blood and bone marrow were purified, cryopreserved, and enriched for CD34⁺ cells as described above. Enriched CD34⁺ fractions were stained with fluorescent antibodies against human CD45, CD34, CD38, Lineage markers (BD PharMingen; CD2 PE-Cy5, 1:20, cat 555328, CD3 PE-Cy5, 1:20, cat 555334, CD4 PE-Cy5, 1:10, cat 555348, CD8 PE-Cy5, 1:50, cat 555368, CD14 PerCP-Cy5.5, 3:100, cat 550787, CD19 PE-Cy5, 1:50, cat 555414, CD20 PE-Cy5, 1:20, cat 555624, CD56 PE-Cy5, 1:10, cat 555517, CD45 APC, 1:50, cat 335790, CD34 BV421, 1:100, cat 562577, CD38 PE-Cy7, 1:50, cat 335790), and propidium iodide. Cells were FACS-purified using a FACS Aria II (Sanford Consortium Stem Cell Core Facility) into hematopoietic stem cell (Lin⁻CD45⁺CD34⁺CD38⁻) and progenitor (Lin⁻CD45⁺CD34⁺CD38⁺) populations directly into RLT lysis buffer (QIAGEN) for RNA extraction followed by RNA-Seq (The Scripps Research Institute Next Generation Sequencing Core) on Illumina HiSeq platforms.

### Bioinformatics Analysis

The analysis code and documentation for the computational analyses are available through Github: https://github.com/ucsd-ccbb/MPN_atlas_methods.

Whole Genome Sequencing (WGS) and Mutation Calling without Matched Normal

Whole genome sequencing of 44 saliva samples was performed at 30× coverage. The samples were distributed among PV (n=5), ET (n=4), MF (n=28), CML (n=3) and non-MPN control individuals (n=4, including 3 healthy volunteers and 1 CLL). In parallel, whole genome sequencing of 43 peripheral blood CD34⁺ stem and progenitor cell enriched samples was performed at 90× coverage for the following sample distribution: PV (n=6), ET (n=4), MF (n=26), CML (n=3) and non-MPN control individuals (n=4, including 1 CLL). WGS analysis was performed on 82 samples, with matching 41 peripheral blood CD34⁺ samples. We performed sequence alignment and variant calling using the Genome Analysis Toolkit (GATK) best practice pipeline. The reference genomes were realigned to the human 1000 genomes v37 (Auton et al., 2015), which contains the autosomes, X, Y and MT but without haplotype sequence or EBV. BWA-mem v.0.7.12. (Li and Durbin, 2009) was used for mapping short reads against the human 1000 genomes v37. Subsequent processing was carried out with SAMtools v.1.1 (Cibulskis et al., 2013; Lai et al., 2016; Li et al., 2009; McKenna et al., 2010), Picard Tools v1.96, Genome Analysis Toolkit (GATK) v2.4-9 (McKenna et al., 2010), which consisted of the following steps: sorting and splitting of the BAM files, marking of duplicate reads, local realignment, indel realignment and recalibration of base quality scores, outputting reads coverage files in bed format for each individual, and calling germline and somatic variants.

Ensemble Variant Calling from Whole Genome Sequencing Data

Short-read sequences from paired blood and saliva sample were each mapped against human genome build GRCh38d1.vd1 using BWA-mem v0.7.17 and sorted by SAMtools (Li et al., 2009). No minimum mapping quality score was required for mapping. Duplicate reads were annotated using Picard MarkDuplicate (Heldenbrand et al., 2019) with a validation stringency set to “STRICT.” Variant calling was performed on the paired mapped reads using four independent variant callers: GATK4 Mutect2 v4.1.4.1 (Heldenbrand et al., 2019), Strelka2 v2.9.10 (Kim et al., 2018), Varscan2 v2.4.3 (Koboldt et al., 2012), and MuSE v.1.0rc (Fan et al., 2016). Any mutation identified by at least 2 of the variant callers was considered genuine. The ensemble variant calling pipeline was validated against 10 previously characterized ICGC PCAWG whole-genome sequenced samples exhibiting over 95% concordance in each sample (ICGC/TCGA Pan-Cancer Analysis of Whole Genomes Consortium, 2020). Each of the variant callers used the gnomAD hg38 dbSNP file for filtering (Lek et al., 2016). For Mutect2, paired reads were allowed to independently support different haplotypes during initial variant calling and the expected frequency of alleles not found in the germline resource was 0.00003125 as per best-practices approach (Heldenbrand et al., 2019). Contamination table and read orientation models were built from the paired samples and was subsequently used for filtering. For VarScan2, the initial variant calling expected a tumor purity of 0.8 and the subsequent filtering required a minimum coverage of 10 reads and at least 3 alternative reads in tumor with a minimum alternative allele frequency of 0.2. For Strelka2 and MuSE, the default setting for whole genome sequence was used to produce a list of raw and filtered variants.

### Analysis of Mutational Signatures and Mutational Patterns

Mutational patterns were generated using SigProfilerMatrixGenerator (Bergstrom et al., 2019) and mutational signatures analysis was performed using our well-established SigProfiler computational framework (Alexandrov et al., 2020). Briefly, the framework identifies the set of mutational signatures that optimally explain the observed mutational patterns without overfitting these mutational patterns. The analysis revealed that clock-like signatures SBS1 and SBS5 were sufficient to recapitulate the patterns observed in MPN samples from both CD34⁺ stem cells and bulk blood.

### Variant Annotation & Filtering

Peripheral blood variants were annotated with Oncotator (Ramos et al., 2015) from a multisample VCF file. We filtered variants using the strategy of Sukhai et al. (2019) to obtain somatic variants from tumor only samples, retained insertions, deletions, and nonsynonymous variants with ExAC, 1000 Genomes, and gnomad population allele frequency <0.002. Variants with ClinVar clinical significance of “benign” were removed. We also removed variants present in three normal controls.

### Structural Variant and Copy Number Analysis

Lumpy (Layer et al., 2014) and Manta (Chen et al., 2016) were used to call SV structural variants. SVs not annotated as imprecise but present in both callers (Jeffares et al., 2017) were annotated and prioritized with AnnotSV (Geoffroy et al., 2018) SVs were subsequently filtered to exclude those present in 1000 Genomes Project and gnomad, and ranked 1-4 by AnnotSV. SVs present in the three normal controls were also removed from all samples.

CNVkit was used to discover somatic copy number variants with the batch command and -m wgs parameter. The three normal controls were pooled together for use as a normal panel. Circos plots of variations were created using circlize (Gu et al., 2014; Zhang et al., 2013).

### RNA-Sequencing Read Preprocessing

RNA-Seq was performed on Illumina's NextSeq 500 sequencer with 150 bp paired-end reads. Sequencing data were de-multiplexed and output as fastq files using Illumina's bcl2fastq (v2.17).

### RNA Editing Analysis

RNA reads were aligned using 2-pass alignment with STAR 2.5.2b 2-pass alignment. Alignment deduplication was performed with Picard MarkDuplicates followed for SortSam. Alignments were then processed sequentially according to GATK best practices for calling RNA-Seq variants with tools SplitNCigarReads, RealignerTargetCreator, IndelRealigner, BaseRecalibrator, PrintReads. Variants were called with HaplotypeCaller and filtered with VariantFiltration for FS<30, QD>2, QUAL>20(79). Mismatches in first 6 base pairs of each read were discarded. Alu sites were identified and kept from RepeatMasker. Non-Aluvariants were further processed: We removed those in repetitive regions based on the RepeatMasker annotation. Intronic sites within 4 bp of splicing junctions were removed. Next, we filtered variants in homopolymer runs. All sites were then kept if there were a minimum of three alternative allele carrying reads and ten total reads and a minimum allele frequency of 0.10. We then identified known RNA editing sites according to RADAR (Ramaswami and Li, 2014) and DARNED (Kiran and Baranov, 2010). For patients without matched whole genome sequencing data, there is a non-zero probability that copy number changes could result in false positive editing sites, but the extensive filtering steps should minimize these instances. To filter mismatches to ADAR specific RNA edits, we kept A to G variants in genes on the positive strand and T to C variants on the negative strand (Kiran and Baranov, 2010; Piskol et al., 2013; Ramaswami and Li, 2014; Ramaswami et al., 2012, 2013). Previously, unreported editing sites were predicted with patient data where there were matching RNA and DNA samples. Notably, we only predicted RNA editing sites with sufficient DNA coverage (>10 reads/site) to compare mismatches and excluded sites that were identified as DNA variants. RNA edits were annotated with Oncotator and further filtered to remove sites that exist in ExAC, 1000 Genomes Project, and dbSNP. Sites were annotated with variant classification (3′UTR, 5′UTR, 5′ Flank, nonsynonymous, synonymous, Silent, Intron, ncRNA, IGR. Differential editing analysis was performed using a Chi-Square test compare the differences in editing in each gene for each variant classification (i.e., MDM2-3′UTR MF versus AN). Significance was set at p<0.05. The contingency table for each test was set up as follows

| | Condition 1 | Condition 2 |
|---|---|---|
| Edited | N sites | N sites |
| Not Edited | N possible sites-N sites | N possible sites-N sites |

N sites is the number of aggregated sites where N possible sites is the number of uniquely edited coordinates within a variant classification*number of samples. Genes with only intergenic differentially editing events were removed. To account for multiple testing, adjusted p values were calculated using the Benjamini-Hochberg procedure and genes with events below an adjusted p value of 0.05 were called significant and retained in the final lists.

### Transcript and Gene Quantification and Differential Expression

Quality control of the raw fastq files was performed using the software tool FastQC (Andrews, S. & Others. FastQC: a quality control tool for high throughput sequence data. (2010). Sequencing reads were aligned to the human genome (hg19) using the STAR v2.5.1a aligner (Dobin et al., 2013). Read and transcript quantification was performed with RSEM (Li and Dewey, 2011) v1.3.0 and GENCODE annotation (genocode.v19.annotation.gtf). The R BioConductor packages edgeR (Robinson et al., 2010) and limma (Ritchie et al., 2015) were used to implement the limma-voom (Law et al., 2014) method for differential expression analysis at both the gene and transcript levels. The experimental design was modeled upon disease and tissue type (approximately 0+disease; approximately 0+tissue; approximately 0+disease+tissue). Significance was defined by using an adjusted p value cut-off of 0.05 after multiple testing correction using a moderated t-statistic in Limma. Genes or transcripts with an adjusted p value of <0.05 (based on the moderated t-statistic using the Benjamini-Hochberg (BH) method for multiple testing correction [27]) were considered significantly differentially expressed (DE) (Benjamini et al., 2001). Functional enrichment of the differentially expressed genes/transcripts was performed using Signaling Pathway Impact Analysis with the Bioconductor package SPIA (Tarca et al., 2009).

Functional enrichment of the differentially expressed genes/transcripts was performed using Signaling Pathway Impact Analysis with the Bioconductorpackage SPIA (Tarca et al., 2009). Gene Set Enrichment Analysis was performed with the Bioconductor package GSVA (Hanzelmann et al., 2013).

### Network Analysis of Differentially Edited Genes

Significantly differentially edited genes were used as seeds for network propagation (Cowen et al., 2017) on the STRING high confidence interactome (Szklarczyk et al., 2015) for three comparisons (AML versus MF, AML versus Aged Normal, MF versus Aged Normal). The most proximal genes to the seed set were identified using a network propagation method, using degree-matched sampling to generate proximity z-scores for each gene in the network. Genes with a z-score >2 were retained in the network and used for visualization and downstream analysis. A graph-based modularity maximization clustering algorithm was used to identify groups of genes within the most proximal genes which were highly interconnected. Genes in the entire network and within each of these clusters were annotated with associated pathways identified by functional enrichment analysis, with the gprofiler tool (Reimand et al., 2007) using the proximal gene set as the background gene list for enrichment of the clusters and the STRING interactome genes as the background for the entire network enrichment.

Network visualization and propagation was performed using Cytoscape (Shannon et al., 2003) and VisJS2jupyter (Rosenthal et al., 2018). The subgraph composed of the most proximal genes is visualized using a modified spring-embedded layout algorithm, modified by cluster membership, so that genes belonging to the same cluster are separated from other clusters. Differential expression log fold change was mapped to the node color, for the significantly differentially expressed genes (FDR <0.05) within the subgraph.

scRNA-Seq Analysis

tSNE visualization: For each cell, read counts per gene were transformed into count probabilities p by dividing by total number of reads detected from that cell. A suitable distance metric defined between any two cells i and j is the Jensen-Shannon divergence (Lin, 1991):

$${JS}_{ij} = H [ \frac{p ( i ) + p ( j )}{2} ] - \frac{1}{2} H [ p ( i ) ] - \frac{1}{2} H [ p ( j ) ] .$$

Lentiviral Overexpression and shRNA Knockdown

Lentiviral human wild-type and mutant ADAR1^{E912A} (pCDH-EF1-T2A-copGFP) and shRNA targeting ADAR1 were produced according to published protocols (Zipeto et al., 2016). All lentiviruses were tested by transduction of 293T cells and transduction efficiency was assessed by qRT-PCR. Lentiviral transduction of primary patient samples was performed at a MOI of 100 to 200. The cells were cultured for 3 to 4 days in 96-well plates (2×10⁵-5×10⁵ cells per well) containing StemPro (Life Technologies) media supplemented with human IL-6, stem cell factor (SCF), Thrombopoietin (Tpo) and FLT-3 (all from R&D Systems) (Abrahamsson et al., 2009; Goff et al., 2013; Jiang et al., 2013; Zipeto et al., 2016). The transduced cells were collected for RNA extraction and cDNA was synthesized according to published methods (Abrahamsson et al., 2009; Goff et al., 2013; Jiang et al., 2013; Zipeto et al., 2016).

Lentiviral Overexpression and shRNA Knockdown

Lentiviral human wild-type and mutant ADAR1^{E912A} (pCDH-EF1-T2A-copGFP) and shRNA targeting ADAR1 were produced according to published protocols (Zipeto et al., 2016). All lentiviruses were tested by transduction of 293T cells and transduction efficiency was assessed by qRT-PCR. Lentiviral transduction of primary patient samples was performed at a MOI of 100 to 200. The cells were cultured for 3 to 4 days in 96-well plates (2×10⁵-5×10⁵ cells per well) containing StemPro (Life Technologies) media supplemented with human IL-6, stem cell factor (SCF), Thrombopoietin (Tpo) and FLT-3 (all from R&D Systems) (Abrahamsson et al., 2009; Goff et al., 2013; Jiang et al., 2013; Zipeto et al., 2016). The transduced cells were collected for RNA extraction and cDNA was synthesized according to published methods (Abrahamsson et al., 2009; Goff et al., 2013; Jiang et al., 2013; Zipeto et al., 2016).

### In Vivo Humanized Mouse Model of MF

Human CD34⁺ cells isolated from MF744 (JAK2 V617F⁺) patient blood were transduced with pCDH lentivirus control or ADAR1-OE lentivirus with a MOI of 100 for 48 hours, followed by intravenous transplantation into adult NSG-S mice (NSG-SGM3), expressing human IL-3, GM-CSF and SCF, 24 hours after 300 cGy of irradiation. Following engraftment at 13 weeks post-transplantation, BM and spleen were collected and processed. Engraftment in BM and spleen of each mouse was analyzed by flow cytometry.

### Generation of Stable Cell Lines

TF1a cells were cultured in RPMI medium supplemented with 10% fetal bovine serum. Cells were transduced with pLKO.1 shScrambled or pLKO.1 shADAR1 lentiviral vectors, respectively. Stable knockdown was confirmed via Western Blot and cells were expanded.

### INFα Treatment

Cells were treated with a single dose of IFNα (R&D Systems) at 10 ng/ml 16 hrs before harvest and analysis.

### Protein Extraction and Western Blot

Cells were harvested and washed twice with ice-cold PBS before being resuspended in lysis buffer (20 mM Tris pH7.5, 150 mM NaCl, 5% Glycerol, 0.5% NP-40, freshly added protease inhibitor cocktail). Cells were lysed on ice for 15 min, then centrifuged at 16,000*g for 10 min to get rid of insoluble material. Supernatant was boiled in 5×SDS buffer (250 mM Tris pH6.8, 40% Glycerol, 10% SDS, 0.01% Bromphenol Blue, 5% beta-mercaptoethanol) for 5 min, then resolved by SDS-PAGE and transferred onto PVDF membranes. Membranes were incubated with 5% BSA in TBS-T for blocking and probed with primary and secondary antibodies diluted in 5% BSA in TBS-T.

APOBEC3C lentiviral Vectors

Lentiviral human wild-type APOBEC3C (pCDH-EF1-T2A-copGFP) was cloned by Eton Biosciences. Mutant APOBEC3C^{E68Q} lacking catalytic activity was created by introducing a single G-to-C point mutation in the active site of APOBEC3C using the QuikChange II site-directed mutagenesis kit (Agilent). All lentiviruses were tested by transduction of 293T cells and transduction efficiency was assessed by qRT-PCR. Lentiviral transduction of primary patient samples was performed at a MOI of 100 to 200. The cells were cultured for 48 to 72 hours in 96-well plates (2×105-5×105 cells per well) containing StemPro (Life Technologies) media supplemented with human IL-6, stem cell factor (SCF), Thrombopoietin (Tpo) and FLT-3 (all from R&D Systems) (Abrahamsson et al., 2009; Goff et al., 2013; Jiang et al. 2013; Zipeto et al., 2016). The transduced cells were collected for RNA extraction and cDNA was synthesized according to published methods (Abrahamsson et al., 2009; Goff et al., 2013; Jiang et al., 2013; Zipeto et al., 2016), or collected into sterile PBS containing 2% FBS for staining and flow cytometry analysis.

### APOBE3C WT and Mutant Flow Cytometry

A minimum of 200,000 CD34 selected normal mixed donor cord blood cells lentivirally transduced for 48 or 72 hours with pCDH backbone, APOBEC3C and APOBEC3C^{E68Q} mutant were blocked using anti-human FcR blocking reagents (Miltenyi Biotec) and then subjected to the following stains: Dapi for live cell discrimination, CD34-APC (BD Biosciences, Clone 8G12), CD38-PEcy7 (BD Biosciences, Clone HB7), CD3-APCcy7 (Biolegend, Clone 17A2), CD14-PerCPcy5.5 (Biolegend, Clone HCD14), CD19-PE (BioLegend, CloneHIB14). Cells were analyzed on a Miltenyi Biotec MACS Quant flow cytometer. Upon debris, doublet, and dead cell exclusion, samples were analyzed for abundance of each differentiation marker, including hematopoietic stem cells (CD3−CD14−CD19−CD34+CD38−), and hematopoietic progenitors (CD3−CD14−CD19−CD34+CD38+).

### ADAR and APOBEC3C Co-Immunoprecipitation

HEK293T cells were transfected at 90% confluence with either pCDH, ADAR1+pCDH, APOBEC3C-FLAG, or ADAR1+APOBEC3C. Cells were collected after 72 hours into non-denaturing lysis buffer for 30 minutes. Collection of starting material (SM) at this point. Lysate was bound to Anti-FLAG M2 magnetic beads overnight at 4° C. Supernatant was removed and saved to check binding efficiency (flow through, FT). Beads boiled in 1×SDS-2-mercaptoethanol loading buffer and loaded into gel. Gels probed for ADAR1 and β-actin loading control.

### Phosph-STAT3 Flow Cytometry

The samples were incubated with NearIR Live/Dead at 1:1000 at room temperature in the dark for 15 minutes. Samples were then blocked with anti-mouse and anti-human FcR for 20 minutes in the dark at 4 C. Afterward, samples were stained with CD34 BV421 at 1:100 and incubated for 20 minutes in the dark at 4 C. Next, samples were fixed with 0.8% PFA and permeabilized with 1× saponin. Samples were incubated with pSTAT3 FITC at 1:10 overnight. Samples were ran on the MACS Quant 10 Analyzer and analyzed utilizing FlowJo.

### Immunofluorescence

The slides for immunofluorescence were prepared by diluting cells (4×105 cells/ml) in PBS. 200 μl of cells were spotted on microscope slides by cytospin at 1,000 rpm for 10 minutes at room temperature. After cytospin, the slides were transferred into a coplin jar containing ice-cold PBS incubate for 5 min; transferred into ice-cold CSK buffer (10 mM PIPES, pH 6.8; 100 mM NaCL; 300 mM sucrose; 3 mM MgCl2) incubate 1 min; transferred into ice-cold CSKT buffer incubate for 5 min; transferred into ice-cold CSK buffer for 1 min; transferred into 4% paraformaldehyde in PBS incubate for 10 min at room temperature. Immunofluorescence was performed by immersing slides in PB ST (1×PBS with 0.1% Tween-20) for several minutes. Slides were overlaid with 250 μl of blocking solution (1×PBS, 1% fetal bovine serum, 0.1% Tween-20) for 1 hour at room temperature. Blocking solution was removed and 100 μl of primary antibody was added to the cells and incubated for 3 hours at room temperature. The slides were washed 2 times in coplin jars with PB ST for 5 min each at room temperature. Secondary antibody was overlaid to spotted cells for 1 hour in the dark. Slides were washed in coplin jars with PB ST 2× at room temperature. DAPI was added and the slides were sealed with a coverslip. Imaging was performed using an Olympus Fluoview confocal microscope.

### Quantification and Statistical Analysis

Data are shown as mean±SEM in all graphs and statistical differences were calculated using a two-tailed unpaired Student's t test, unless otherwise indicated. p values <0.05 were considered significant. The number of n (number of patients or number of experimental repeats) are indicated in each figure legend, All statistical analyses and plots were produced in GraphPad Prism or R (v3.3.3).

### Figures Example 2:

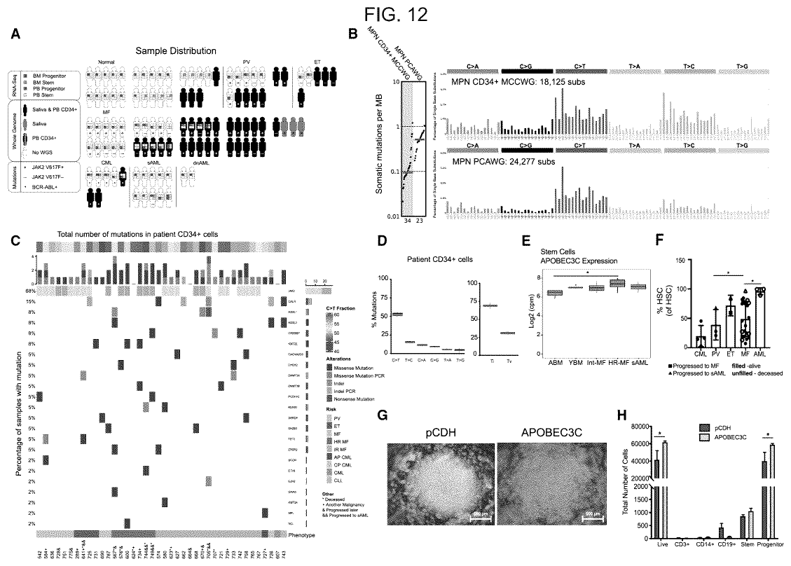

FIG. 12 (or FIG. 1, as used in Example 2)

### MPN Pre-Leukemia Stem Cell Expansion and APOBEC3C Activation

(A) Sample distribution in this study. Samples were distributed among polycythemia vera (PV, n=5), essential thrombocythemia (ET, n=4), myelofibrosis (MF, n=28), chronic myeloid leukemia (CML, n=3), and non-MPN control individuals (n=4, including 3 healthy volunteers and 1 CLL with CALR SNP). In parallel, whole-genome sequencing of 43 peripheral blood samples of a sample distribution of PV (n=6), ET (n=4), MF (n=26), CML (n=3), and non-MPN control individuals (n=4, including 1 CLL with CALR SNP). The somatic mutations were obtained from MPN patient samples (n=37) and non-MPN controls (healthy controls n=3 and CLL with CALR SNP n=1) with matching saliva (30× coverage) and peripheral blood (n=4), shown in solid black). Whole-transcriptomic sequencing (RNA-seq) was performed on 78 samples distributed as follows: PV (n=6), ET (n=2), MF (n=29), CML (n=5), AML (n=12), and non-MPN control individuals (n=24). These samples can further be broken down based on tissue of collection (peripheral blood or bone marrow) and cell types (stem cells and progenitor). In summary, from 54 subjects and 24 non-MPN controls, 113 samples were represented in the RNA sequencing analysis.

(B) Mutational burden of single point mutations (log-scaled). Each dot represents the number of substitutions per megabase in an individual MPN sample. Red lines reflect median numbers. Mutational profiles of substitutions are shown using six subtypes: C>A, C>G, C>T, T>A, T>C, T>G. Underneath each subtype are 16 bars reflecting the sequence contexts determined by the four possible bases 5′¢ and 3¢′ to each mutated base. Average contributions of the two clock-like signatures across PCAWG MPN and MCCWG MPN samples are shown in different colors.

(C) Mutations in 69 MPN-associated genes (Grinfeld et al., 2018) in peripheral blood divided by MPN disease stage. Clinical-grade confirmation of JAK2 V617F mutation was marked as light yellow in MPN patients.

(D) boxplot depicting the number of somatic mutations in peripheral blood or saliva based on transitions (Tis) or transversions (Tvs). Both somatic and germline variants were included.

(E) A boxplot depicting the expression levels of APOBEC3 in ABM, YBM, intermediate-risk myelofibrosis (Int-MF), high-risk myelofibrosis (HR-MF), and sAML stem cell populations using normalized RNA-seq. APOBEC3C expression was illustrated for each stem cell sample compared with ABM normal controls. *p<0.05.

(F) Comparison of the HSC percentage in MPN samples by flow cytometry (CML n=4, PV n=3, ET n=2, MF n=23 and AML n=3).

(G) A representative bright-field microscopic image of cord-blood CD34 cells lentivirally transduced with APOBEC3C compared with a lentiviral backbone control (left).

(H) Flow-cytometry analysis of cord-blood CD34⁺ cells 48 h after lentiviral transduction. Error bars show SEM and significance determined by two-way ANOVA.

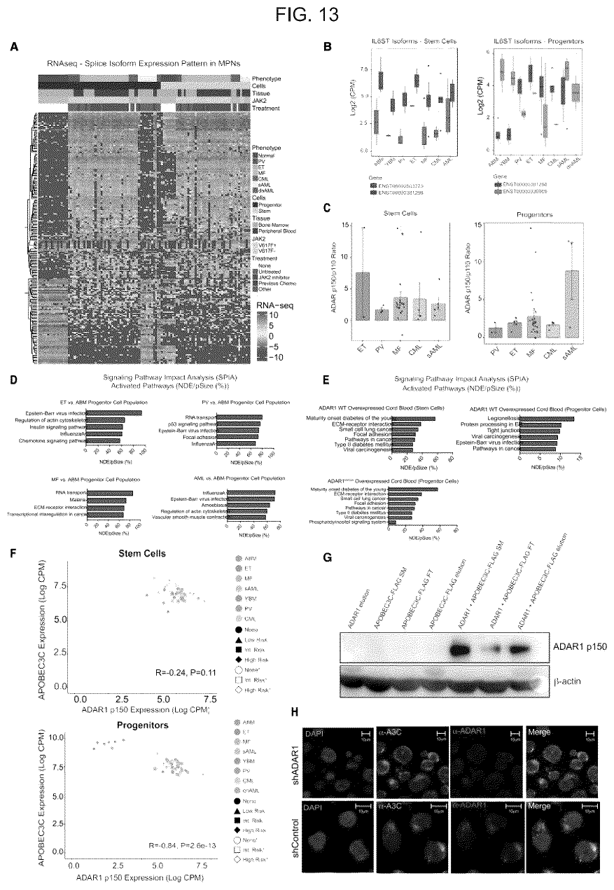

FIG. 13 (or FIG. 2, as used in Example 2)

### Isoform Switching Favoring ADAR1p150 Expression Drives Pre-LSC Evolution

(A) Heatmap of RNA-seq expression of splicing isoforms for the top 1% of genes ranked by variance. Annotation for each sample is presented as a stack of colored bars representing phenotype, cell type, source tissue, mutation status, and the treatment type (for MF samples only). Samples without a known JAK2 V617F mutation status are colored in gray.

(B) A boxplot representing the internally normalized expression of IL6ST isoforms (ENST00000381298 and ENST00000503773 in stem cells) and (ENST00000381298 and ENST00000336909 in the progenitors) in each MPN phenotype. Black dots represent expression values in lowest 2.5% or highest 97.5% of the distribution.

(C) Ratio of ADAR1 isoforms (p150/p110) in each MPN disease type using RNA-seq expression data from stem cells and progenitors.

(D) Signaling pathway impact analysis (SPIA) was performed for ET, PV, MF, and AML compared to ABM progenitors. Listed are the top 5 activated pathways based on the NDE (number of differentially expressed genes per pathway)/pSize (number of genes in the pathway) in percentages.

(E) SPIA in cord blood lentivirally transduced with ADAR1 WT (top) or RNA deaminase-deficient mutant ADAR1^{E912A} (bottom) compared to pCDH backbone controls (n=3). Listed are the top 6 activated pathways based on the NDE/pSize in percentages.

(F) Correlation of normalized and Log 2-transformed counts per million (CPM) data for APOBEC3C with ADAR1 p150 isoform in stem cells (top) and progenitors (bottom). Points are colored by phenotype. The MF risk group is indicated by point shape with open shapes representing deceased patients.

(G) Western blot probed for ADAR1 p150 after co-immunoprecipitation with ADAR1 and APOBEC3C-FLAG.

(H) Colocalization of APOBEC3C and ADAR1 in TF1a cells. Immunofluorescence of anti-APOBEC3C (green) and anti-ADAR1 p150-specific (red) antibodies in TF1a shADAR1 and TF1a shControl knockdown cells demonstrate a colocalization (yellow) of APOBEC3C and ADAR1 p150 proteins in the shControl cells. TF1a shADAR1 cells show ablation of ADAR1 protein.

FIG. 14 (or FIG. 3, as used in Example 2)
A-to-I Hyper-Editing Distinguishes Pre-LSC and LSC from Normal Progenitors

(A) Violin plot of overall RNA editing variant allele frequency (VAF) by MPN subtype and YBM and ABM controls. The overall VAF is statistically significantly elevated in PV, ET, MF, CML, de novo AML, and sAML primary patient samples compared to the normal ABM counterpart.

(B) Correlation of mean A-to-I RNA editing level with normalized and Log 2-transformed ADAR1 p150 isoform CPM level in both stem cells (square) and progenitors (triangle). Each color represents a distinct MPN disease stage.

(C) Boxplots comparing VAF of each MPN progenitor subtype and YBM and ABM controls stratified by genomic region.

(D) Statistical comparison of data from (C). The p value values are derived from comparing the VAFs of each MPN stage and ABM at each variant classification by the Kolmogorov-Smirnov test.

(E) Top 25 ranked genes by occurrence of nonsynonymous RNA edit mutations broken down by known non-A/u and Alu region and previously unknown non-Alu and Alu regions stratified by MPN phenotype, treatment, and cell type.

(F) Normalized Log 2 transformed RNA-seq expression data for CDK13 in the stem and progenitor population plotted by MPN phenotype. The results of t tests (ns=not significant; *p<0.05; **p<0.01; ***p<0.005) between each phenotype and the ABM) group are shown.

(G) Expression of normalized ADAR1 RNA-seq expression data compared with expression normalized CDK13 in stem (left) and progenitor (right) populations. The significance of the Pearson correlation (relative to R=0) is shown along with a trendline of the data.

(H) Colocalization of CDK13 and ADAR1 in sAML cells by immunostaining of anti-CDK13 (green) and anti-ADAR1 (red) antibodies.

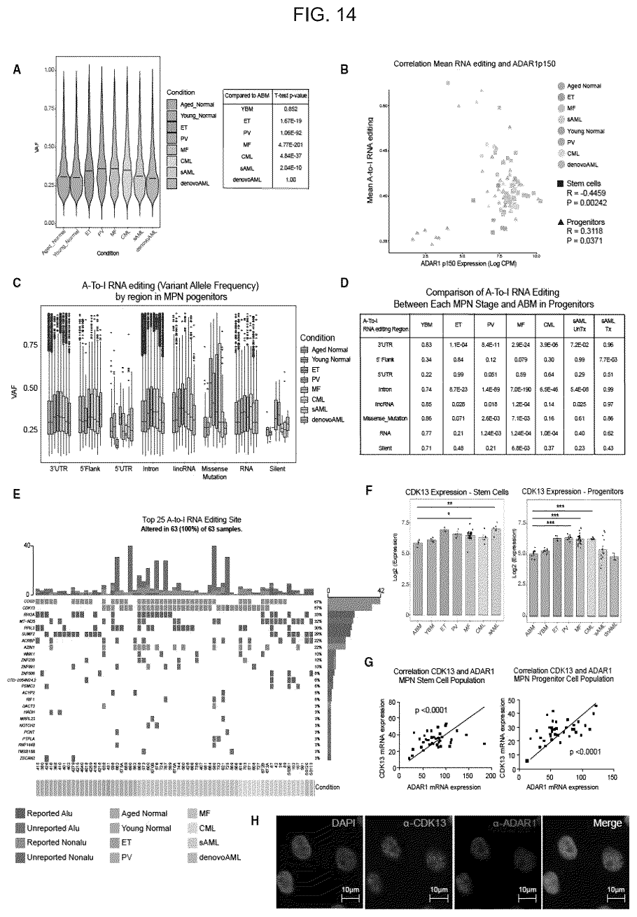

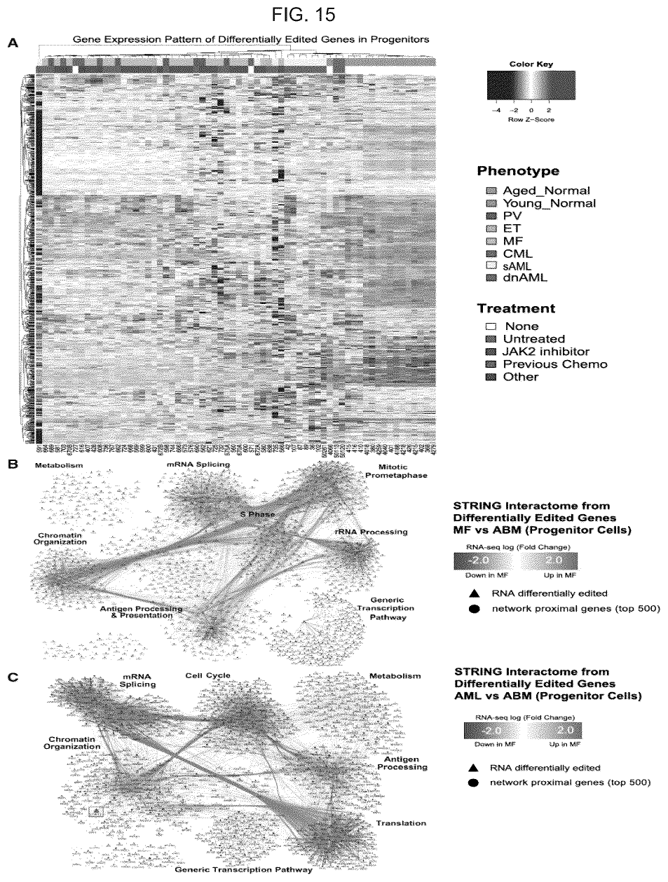

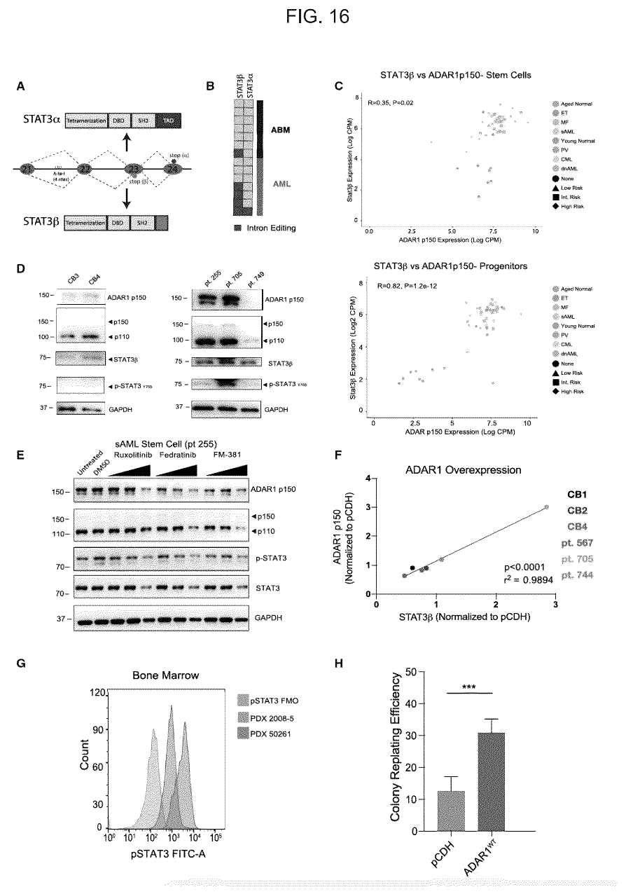

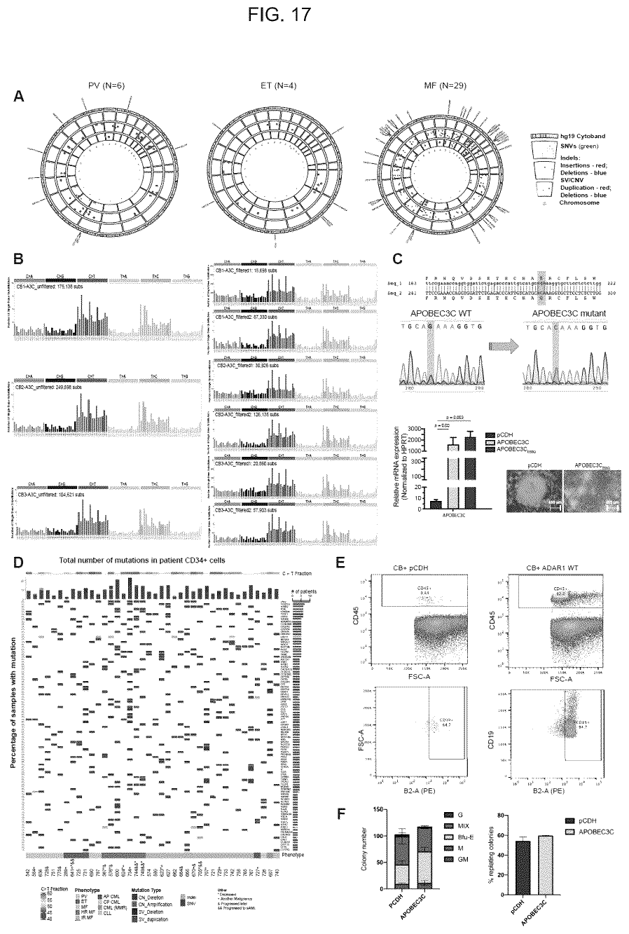

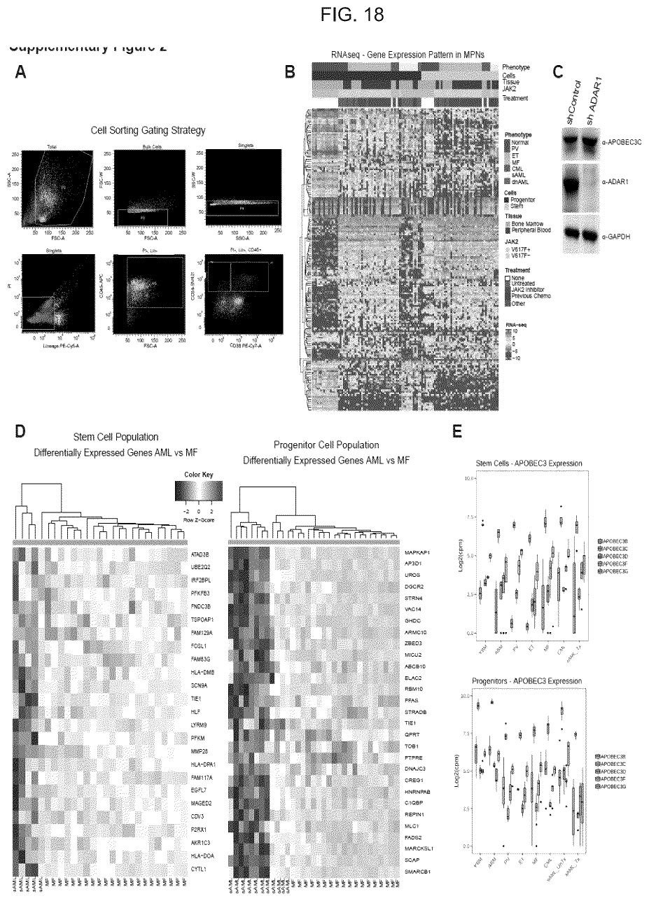

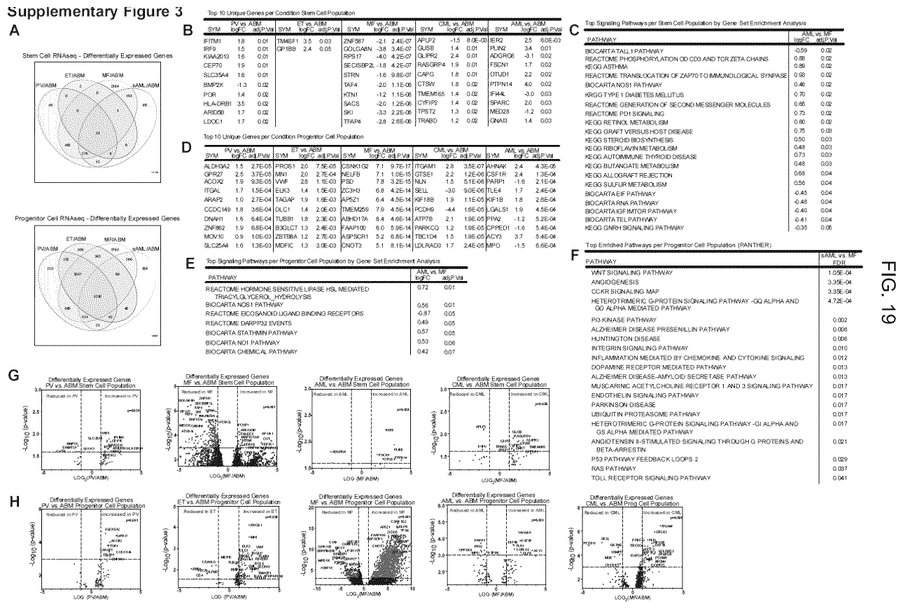

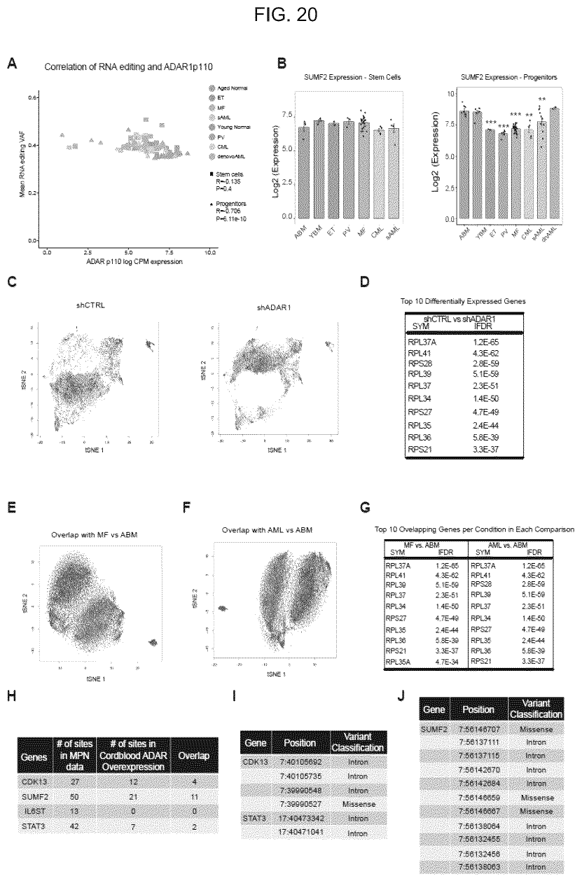

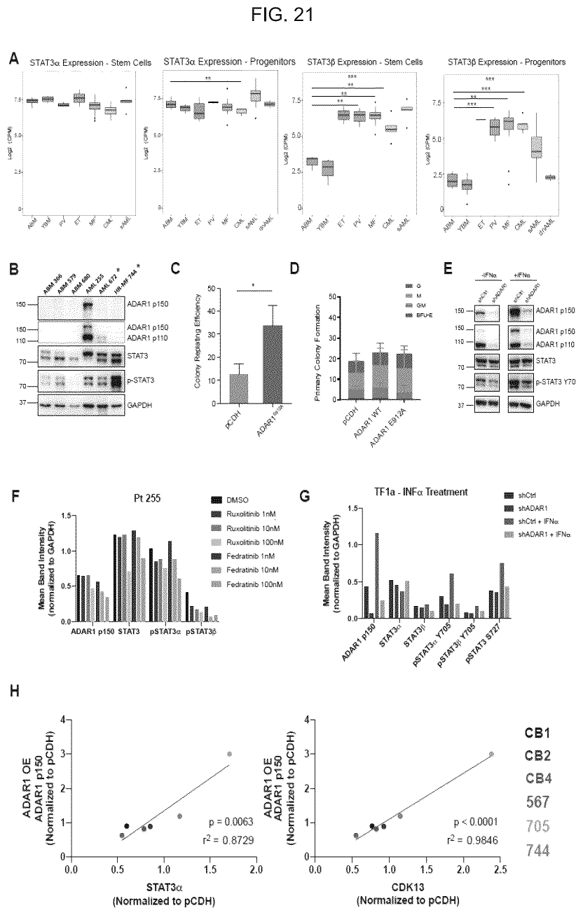

FIG. 15 (or FIG. 4, as used in Example 2)
The RNA Editome Distinguishes Pre-LSCs from LSCs

(A) Heatmap based on gene expression Z scores of 1,295 differentially edited genes across all comparisons with aged bone marrow (ABM).

(B and C) Network analysis of differentially edited genes between (B) normal aged samples (ABM) and MF and (C) normal aged samples (ABM) and AML. In MF (B), out of the 834 significantly differentially edited genes, 690 were found in the interactome and used as seeds for network propagation on the STRING high confidence interactome. In AML (C), out of the 757 significantly differentially edited genes, 642 were found in the interactome and used as seeds for network propagation on the STRING high confidence interactome. Differential expression log fold change is mapped to node color: blue, significantly down in MF compared to ABM; red, significantly up in MF compared to ABM. Gray nodes were not significantly differentially expressed (false discovery rate (FDR)<0.05).

FIG. 16 (or FIG. 5, as used in Example 2)

### ADAR1-Induced STAT3 Intronic Editing and Splice Isoform Switching in LSCs

(A) Diagram of STAT3 isoform generation by intronic RNA editing of STAT3 transcripts.

(B) Intronic A-to-I RNA editing locations (Goldberg et al., 2017) in ABM and AML as determined by RNA-seq analysis.

(C) Correlation of normalized Log 2-transformed CPM data of the STAT30 isoform and the ADAR1 p150 isoform in stem cells and progenitors of ABM, YBM, MPN, and AML samples. The MF risk group is indicated by the diamond shape.

(D) Western blot analysis of cord-blood CD34⁺ cells (left, n=2), high-risk MF (pt. 705 and 749), and sAML (pt. 255) CD34⁺ cells (right, n=3).

(E) Western blot analysis of sAML (pt. 255) CD34⁺ cells treated with FDA-approved JAK2 inhibitors (ruxolitinib and fedratinib) compared with a JAK3 inhibitor (FM-381) at concentrations of 1, 10, and 100 nM.

(F) Correlation of ADAR1 p150 expression with the expression of STAT30 isoform. The CD34⁺ cells from cord-blood (n=3), sAML, and high-risk MF samples (n=5) were transduced with pCDH, ADAR1-overexpressing vectors. The relative gene expression was measured by RT-qPCR and normalized to HPRT values.

(G) pSTAT3 levels measured by flow cytometry in CD34⁺ populations of two sAML patients (2008-5 and 50261).

(H) Self-renewal capacity, as measured by colony replating assays, in MF CD34⁺ cells transduced with pCDH backbone or ADAR1 WT.

### Example 3: Compositions and Methods for Using Purified Human RNA Editing Enzymes

This Example describes the purification and production of human functional anti-viral RNA editing enzymes, ADAR1 (adenosine deaminase associated with RNA1), and related lentiviral vectors, editing reporters and compounds as well as methods of use relating to the discovery of anti-viral compounds, stem cell expansion, inhibition of cancer stem cells and selective RNA base editing as well as inhibition of RNA viruses including SARS CoV-2 and retroviruses.

In particular, this Example provides methods to produce large amounts of recombinant human full length ADAR1, Z alpha binding domain deleted ADAR1, and the catalytic domain of ADAR1. Also, methods of use are described for our ADAR1 overexpression and shRNA knockdown lentiviral vectors, stable lentiviral overexpression cell lines and shRNA knockdown cell lines with a lentiviral A-to-I editing selective luciferase GFP reporter for detection of ADAR1 substrates, including RNA viruses, such as SARS-CoV-2, HIV and influenza A and B, as well as compound discovery and validation. These methods are engineered to produce a large amount of the desired quantify of human ADAR1 product with a robust ability to catalyze adenosine deamination using a desired substrate. The disclosure also provides methods of preparing ADAR1 mutant enzymes that lack the Z alpha binding domain and those that have only the catalytic domain; as well as methods of use thereof in improving the biosynthesis of ADAR1-specific stimulatory and inhibitory compounds and lentiviral vectors for potential clinical use.

### BACKGROUND

Anti-viral deamination by ADAR1 induces adenosine to inosine (A-to-I) editing that restricts replication of RNA viruses, such as coronaviruses and influenza as well as retroviruses like HIV. Targeted base editing by ADAR1 has also emerged as a potent means to introduce single nucleotide changes in RNA to alter splice acceptor sites and transcript susceptibility to microRNA targeting and ultimately changes in translation. Moreover, Z alpha DNA binding by ADAR1 may alter the epigenome within select Alu-containing regions while Z alpha RNA binding may induce changes in transcript stability. Hyper-editing by ADAR1 induces RNA alterations in survival and stem cell transcripts, lncRNA and primary microRNA, primarily in the context of double stranded RNA loops formed by Alu sequences, and promotes therapeutic resistance in cancer stem cells as well as self-renewal of normal human hematopoietic stem cells (Jiang et al PNAS 2013; Zipeto et al Cell Stem Cell 2016; Crews et al Nature Communications 2017; Jiang et al Cancer Cell 2019). As an innate immune anti-viral deaminase, ADAR1 is transcriptionally activated following inflammatory cytokine signaling through JAK2/STAT and interferon α, β and γ signaling. Thus, selective JAK2 as well as STAT3 inhibition prevents ADAR1 activation.

Herein we describe methods to purify human full length ADAR1, Z alpha domain deleted ADAR1 and the catalytic domain of human ADAR1 as well as the production of lentiviral ADAR1 Nano-luc reporter transduced interferon responsive and unresponsive cell lines with ADAR1 overexpression and shRNA knockdown for the purposes of screening for anti-viral compounds capable of inhibiting replication of RNA viruses and retroviruses. We also describe methods to identify ADAR1 antagonists, including lentiviral ADAR1 shRNA knockdown, mutant and Z alpha domain deleted ADAR1 vectors, capable of inhibiting cancer stem cells. Methods are described to detect ADAR1 agonists, including lentiviral ADAR1 overexpression vectors, capable of enhancing stem cell survival, self-renewal and anti-viral activity.

Provided are methods of eradicating cancer cells, or cancer stem cells, comprising an ADAR1 inhibiting agent that significantly reduces ADAR1 Nano-luc reporter activity in cell lines and in human cancer stem cell assays. In alternative embodiments, the ADAR1 inhibiting agent comprises: a JAK2 inhibitor, for example, fedratinib, a STAT3 inhibitor, 8-aza-adenosine, or a nucleoside analog or integrase inhibitor such as, raltegrovir or dolutegravir, or any combination thereof.

In alternative embodiments, the ADAR1 inhibiting agent comprises a lentiviral shRNA ADAR1 knockdown vector, or a lentiviral ADAR1 mutant vector, or a lentiviral ADAR1 Z alpha domain deleted vector, or an interferon inhibitory compound.

In alternative embodiments, provided are methods to identify an ADAR1 agonist using ADAR1 Nano-luc reporter interferon responsive and interferon cell lines. In alternative embodiments, lentiviral ADAR1 or lentiviral ADAR1 shRNA are used as ADAR1 inhibiting agents. In alternative embodiments, recombinant human full length ADAR1, or recombinant human ADAR1 catalytic domain, or recombinant human Z alpha domain deleted ADAR1, is used.

In alternative embodiments, a lentiviral JAK2 overexpression vector is used.

In alternative embodiments, provided are stably transduced human non-interferon responsive cell line containing lentiviral ADAR1 overexpression vector and Nano-luc reporter for the purpose of detecting RNA virus inhibition, including SARS-CoV-2 and influenza A and B.

In alternative embodiments, provided are stably transduced human interferon responsive cell line containing lentiviral ADAR1 overexpression vector and Nano-luc reporter for the purpose of detecting RNA virus inhibition, including SARS-CoV-2, influenza A and B or HIV, following infection with an RNA virus or retrovirus.

In alternative embodiments, provided are methods of inhibiting SARs-CoV-2 or other RNA viruses or retroviruses based on lentiviral ADAR1 overexpression and IV administration of lentiviral ADAR1 transduced stem cells including cord blood CD34+ cells or mesenchymal stromal cells.

In alternative embodiments, provided are methods for inhibiting SARs-CoV-2 or other RNA virus or retrovirus replication based on ADAR1 catalytic domain nanoprotein delivery with liposomes by intravenous administration or inhalation.

In alternative embodiments, provide are methods inhibiting SARs-CoV-2 or other RNA virus or retrovirus replication based on ADAR1 full length nanoprotein delivery with liposomes intravenous administration or inhalation.

In alternative embodiments, provided are methods for inhibiting SARs-CoV-2 or other RNA virus or retrovirus replication based on ADAR1 Z alpha domain deleted nanoprotein delivery with liposomes intravenous administration or inhalation.

### Methods

### Human ADAR1 Catalytic Domain (CD) Purification Protocol

Yeast Strain: Saccharomyces cerevisiae BJ2168 (From Goode lab/Expression
vector: pEG(KT) (URA/LEU minus) (From Zakarian lab/Princeton)

### Gene: Codon Optimized ADAR1-CD (GenScript)

### Yeast Growth Media (1 Liter)

Minimal Selection Media (URA/LEUMinus Growth Media): 100 mM Potassium Phosphate pH 6.0, 6.7 g Yeast Nitrogen Base, 1.92 g Synthetic Amino Acid Drop-Out Mix (Minus URA/LEU), 5.0 g Ammonium Sulfate, 10.0 g Succinic Acid, 2% Glycerol, 3% Lactic Acid, 2% Raffinose. pH media to pH 6.0 using NaOH pellets and sterilize using 0.22 um filter.
5× Induction Media: 50 g/L Select Yeast Extract, 100 g/L Bacto-Tryptone, 10% D(+)-Galactose. Filter media using 0.22 um sterile filter.

### Purification Buffers

### Yeast “Popcorn” Buffer: 20 mM Hepes pH 8.0, 150 mM NaCl

Add fresh 1 mM PMSF and 1 Roche protease inhibitor cocktail pill

### YeastBuster Lysis Buffer (Novagen)

Add 1×THP, 1 mM DTT, 1 mM PMSF, 1 Roche protease cocktail pill

### GST-Pulldown Buffers

### GST Binding Buffer: 20 mM Hepes pH 8.0, 150 mM NaCl, 0.1% Triton X100, 5% Glycerol, 1 mM DTT

Add fresh 1 mM PMSF and 1 Roche protease inhibitor cocktail pill High-Salt Wash Buffer: 20 mM Hepes pH 8.0, 500 mM NaCl, 0.1% Triton X100, 5% Glycerol, 1 mM DTT

### Low-Salt Wash Buffer: 20 mM Hepes pH 8.0, 75 mM NaCl, 0.1% Triton X100, 5% Glycerol, 1 mM DTT

HiTrap Heparin column (GE) Buffers
Buffer A: 20 mM Hepes pH 8.0, 75 mM NaCl, 5% Glycerol, 1 mM DTT, 0.22 um filtered
Buffer B: 20 mM Hepes pH 8.0, 1M NaCl, 5% Glycerol, 1 mM DTT, 0.22 um filtered Dialysis Buffer/Protein Storage Buffer: 20 mM Hepes pH 8.0, 150 mM NaCl, 5% Glycerol, 1 mM DTT

### Superdex 200 10/300 GL (GE) Buffer

Buffer A: 20 mM Hepes pH 8.0, 150 mM NaCl, 5% Glycerol, 1 mM DTT, 0.22 um filtered

### Yeast Growth Procedure

1.) In a sterile 150 mL baffled flask, inoculate 15 mL of Minimal Selection Media (URA/LEU minus media) with a large, single yeast colony picked from a freshly streaked URA/LEU minus plate. Grow culture overnight @ 30° C. in shaker set to 250 RPM. Note: This protocol is designed for the preparation of 3 liters of yeast culture.
2.) The next day, inoculate 800 mL of Minimal Selection Media in 2-liter baffled flasks with 1.5 mL of starter culture each. Grow cultures overnight @ 30° C. in shaker set to 250 RPM.
3.) The next morning, use a Spectronic 200 spectrophotometer to measure the optical density (OD) of the cultures using a wavelength of 600 nm. For ideal gene expression, the OD600 nm should be between 1.0 and 2.0. To induce protein expression, add 200 mL of 5× Induction Media to each flask. Allow yeast to grow @ 30° C. for 24 hours in shaker set to 250 RPM.
4.) Harvest yeast by spinning cultures down @ 5K RPM for 10 minutes using 500 mL spin bottles. Repeat this step until all of the culture is pelleted.
5.) Discard the supernatant and combine pellets by resuspending them in Yeast “Popcorn” Buffer. First, resuspend pellets by vortexing them in 20 mL of buffer. Next, transfer yeast into a 50 mL conical tube, and re-pellet the yeast by using a table-top centrifuge @ 5K RPM for 10 minutes. Discard wash and save pellet.
6.) Resuspend pellet in Yeast “Popcorn” Buffer containing 1 mM PMSF and 1 Roche protease inhibitor cocktail pill. For resuspension, use half the pellet volume and fully resuspend the yeast by vortexing (e.g. Use 5 mL of popcorn buffer for a 10 mL wet yeast pellet). Note: Roche protease inhibitor cocktail pills are pre-solubilized in popcorn buffer before use hereon in through this protocol.
7.) Lastly, make yeast “popcorn” by adding the yeast to liquid nitrogen drop-by-drop in a 50 mL conical tube. Store popcorn @ −80° C. for long-term storage.

### Yeast Lysis Protocol

1.) Begin lysis protocol by pre-chilling ceramic mortar and pestle. To do so, place mortar in a bucket containing dry ice. Make sure to completely cover the sides of the mortar with dry ice. Additionally, fill the mortar with liquid nitrogen, and allow the liquid nitrogen to dissipate completely before use. The pestle and spatula should also be pre-chilled by submerging them in liquid nitrogen prior to Step 2. Note: Always use blue cryogenic gloves when handling liquid nitrogen.
2.) Next, place a small amount of yeast popcorn in the mortar and begin grinding by carefully maneuvering the pestle in a circular motion until a very fine powder is formed. This may take up to 10 minutes per round of grinding.
3.) Transfer ground yeast material, or “powder”, to a new 50 mL conical tube. Yeast powder can be stored at −80° C. before commencing the next step if needed.
4.) Thaw out yeast powder at room temperature. While the powder is thawing, activate 100 mL of YeastBuster Lysis Buffer. To activate the buffer, add 1 mL of 100×THP, 1 mM DTT, 1 mM PMSF, 100 uL cyanase (50U/uL), and 1 Roche protease inhibitor cocktail pill.
5.) Resuspend the powder in all 100 mL activated Yeast Buster Lysis Buffer. DO NOT SHAKE OR VORTEX SAMPLE AS THIS WILL DAMAGE YOUR PROTEIN AND CAUSE FOAM TO FORM!
6.) Place sample to a 500 mL beaker with a stir bar and allow it stir at 100 RPM @ room temperature for 1 hour.
7.) Complete the lysis protocol by douncing the sample 30 times in a pre-chilled dounce homogenizer. This can be done in a piece-wise fashion douncing 15 mL of sample at a time.
8.) Lastly, pellet the unbroken cells and cell debris by spinning extract @ 15K RPM for 25 minutes in a chilled centrifuge. Note: Make sure to use tubes that will withstand these spin speeds (e.g. Nalgene centrifuge tubes).
9.) Discard the pellet and transfer the supernatant containing your protein to new 50 mL conical tubes. Measure the protein concentration of the extract and determine the total amount of protein in sample. Flash freeze extract using liquid nitrogen for later use. Store frozen extracts at −80° C.

### GST-ADAR1-CD Purification Protocol

1.) Begin by thawing protein extracts in room temperature preferably in water. Once thawed, dilute the extract in GST Binding Buffer to a total volume of 500 mL. Add fresh 1 mM DTT, 1 mM PMSF, and 1 Roche protease inhibitor cocktail pill to final diluted extract solution (Starting Material). Ideally, the final protein concentration of the extract will be between 1-2 mg/mL.
2.) While extracts are thawing out, pre-equilibrate GST resin with 150 mL (3 CV) of ice-cold GST Binding Buffer.
3.) Next, pass extract over the GST resin. Keep extract and flow through on ice throughout the entire process as this step takes several hours to complete. Additionally, save 100 uL of starting material and flow through for anti-ADAR1 or anti-GST Western blot analysis.
4.) After passing through the extract, beginning washing the GST resin with the following wash buffers
   a.) 100 mL GST Binding Buffer
   b.) 100 mL High Salt Wash Buffer
   c.) 100 mL GST Binding Buffer
   d.) 100 mL Low Salt Buffer
5.) After the last wash, remove the stop-cock and replace it with a small yellow cap and seal it well with parafilm.
6.) Add 50 mL of Low Salt Buffer, carefully resuspend the GST resin, and transfer all of the beads evenly into 4×50 mL conical tubes (˜25 mL/each tube).
7.) Fill each tube up to 50 mL with Low Salt Buffer containing 1 mM DTT. The final elution volume is 150 mL (approximately 3 CV).
8.) Add 300 uL TEV protease to each tube and incubate overnight at 4° C. rotating gently.
9.) The next day, transfer all of the GST resin and solution back into the purification column. Release/open the top screw cap first to release the internal pressure of the column. Then, carefully open the bottom screw cap and catch the TEV-eluted protein in a 250 mL beaker on ice. Save 160 uL of elution for protein concentration measurement and Coomassie Blue stain analysis.
10.) Proceed with purification by pumping the elution over 5 mL HiTrap Heparin columns (GE) attached in tandem to an AKTA Pure system (FPLC). Use the method script written on the AKTA Pure system. Note: The milligram quantity obtained from Step 9 will determine how many Heparin columns will be needed to capture all ADAR1-CD protein in the sample.
11.) Determine the separation of ADAR1-CD and other contaminating proteins including TEV protease by visualizing Heparin fractions on a SDS-PAGE gel and staining it with Coomassie Blue stain. Note: Determining the separation of proteins is critical before moving on to the next step.
12.) Collect FPLC fractions that contain ADAR1-CD, but not other contaminating proteins, and transfer them to a 50 mL conical tube on ice.
13.) Since the sample is in high salt (−300 mM), the sample needs to be dialyzed in Dialysis Buffer using dialysis tubing that is pre-equilibrated in 1 mM EDTA pH 8.0 in dH20. Make sure that the dialysis tubing has a MWCO no larger than 10-14 kDa. Dialyze overnight at 4° C. in 3 liters of buffer stirring gently with a stir bar.
14.) The next morning, carefully transfer the sample to a new 50 mL conical tube on ice. At this point, the protein is ready to concentrate using a Millipore Amicon-Ultra centrifuge spin column with a MWCO of 10-14 kDa. Spin sample at 5,000×g for 20 minutes. After 20 minutes, take the sample out of the centrifuge and carefully pipette the protein solution to prevent undesired protein aggregation. Continue to spin sample until a final volume of 500 uL is achieved.
15.) Measure the protein concentration of the concentrated sample, save 20 uL for further SDS-PAGE analysis, and flash freeze the rest of the sample in liquid nitrogen. The sample is now ready for downstream applications. Note: Alternatively, the sample can be applied to a Superdex200 10/300 GL gel filtration column after the concentration step to further purify the protein.

### Nano-Luciferase Reporter Design

### In Vivo Visualization of ADAR1 Editase Activity Via Nano-Luc Reporter

### Transduction

K562 cells from ATCC were initially transduced with control, ADAR1 WT, or ADAR1 E912A mutant vectors and maintained stably. These stable lines were then co-transduced with equal MOI of ADAR1 NanoLuc reporter lentivirus. Cells were then sub-cultured and maintained stably before transplantation into mice.

### Transplantation and Imaging

Immunocompromised RAG2−/−yc−/− mice were bred and housed in the Sanford Consortium vivarium per IACUC-approved protocol. Neonates (P2-P3) were transplanted intrahepatically with 100,000 K562 cells transduced with either pCDH, ADAR1 WT, or ADAR1 E912A vectors and ADAR1 NanoLuc reporter (all). Mice were monitored and weighed weekly after P21. Mice with >20% weight reduction (approximately 7 weeks old) compared to non-transplant control were imaged by IVIS lumina imaging system. Promega Nano Luc substrate was prepared at 40× (sterile PBS) and administered intraperitoneal at a volume (ul) equivalent to 10 times mouse weight (g). Mice were euthanized after imaging.

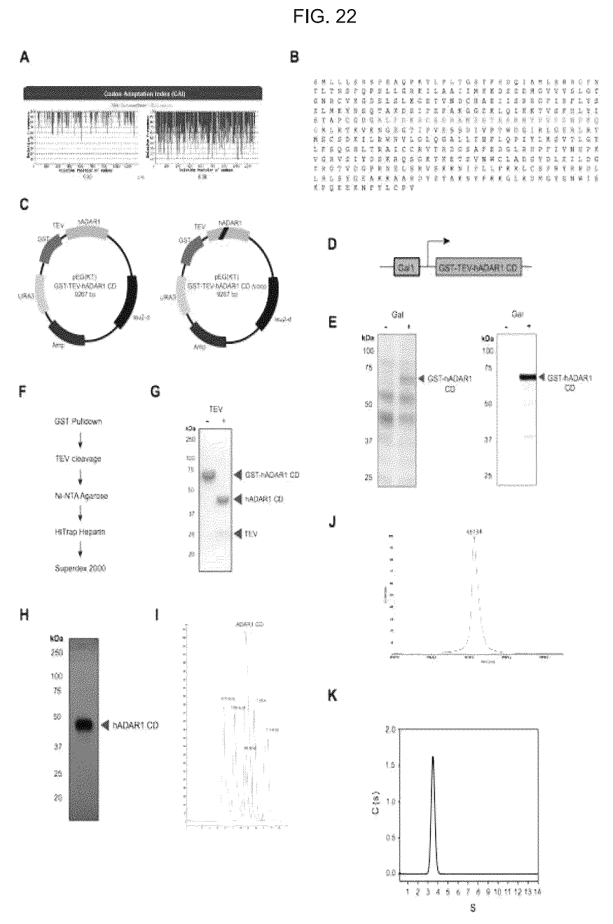

FIG. 22: Expression and Purification of recombinant human ADAR1 Catalytic Domain (hADAR1 CD) in BJ2168 yeast expression system. (A) hADAR1 CD codon optimization for expression in yeast. (B) hADAR1 CD amino acid sequence. Colored amino acids have been deleted in Δloop construct. (C) pEG(KT) GST-TEV-hADAR1 CD and pEG(KT) GST-TEV-hADAR1 CD Δloop vector maps. (D) Schematic representation of Galactose-inducible expression system. (E) Coomassie Blue stain and α-ADAR1 Western Blot confirming Galactose-inducible expression of GST-tagged hADAR1 CD. (F) Workflow showing steps involved in protein purification from yeast cell extract. (G) Coomassie Blue stain showing successful cleavage of GST tag by TEV enzyme. (H) Silver stain demonstrating purity of the hADAR1 CD protein product after final purification step. (I) Size Exclusion Chromatography of purified hADAR1 CD using a Superdex200 10/300 GL gel filtration column. (J) Protein mass determination of purified hADAR1 CD protein product via mass spectrometry. (K) Analytical Ultracentrifugation of purified hADAR1 CD demonstrating purity of the final protein product.

FIG. 22B illustrates
SEQ ID NO: 1
S M L L L S R S P E A Q P K T L P L T G S T F H D Q I A M L S H R C F N
T L T N S F Q P S L L G R K I L A A I I M K K D S E D M G V V V S L G T
G N R C V K G D S L S L K G E T V N D C H A E I I S R R G F I R F L Y S
E L M K Y N S Q T A K D S I F E P A K G G E K L Q I K K T V S F H L Y I
S T A P C G D G A L F D K S C S D R A M E S T E S R H Y P V F E N P K Q
G K L R T K V E N G E G T I P V E S S D I V P T W D G I R L G E R L R T
M S C S D K I L R W N V L G L Q G A L L T H F L Q P I Y L K S V T L G Y
L F S Q G H L T R A I C C R V T R D G S A F E D G L R H P F I V N H P K
V G R V S I Y D S K R Q S G K T K E T S V N W C L A D G Y D L E I L D G
T R G T V D G P R N E L S R V S K K N I F L L F K K L C S F R Y R R D L
L R L S Y G E A K K A A R D Y E T A K N Y F K K G L K D M G Y G N W I S
K P Q E E K N F Y L C P V

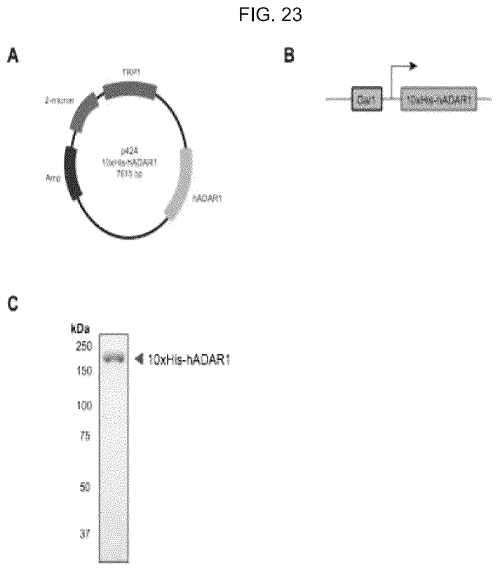

FIG. 23: ADAR1 full length purification scheme and product.

Human ADAR1 amino acid sequence is (SEQ ID NO: 2):
MNPRQGYSLSGYYTHPFQGYEHRQLRYQQPGPGSSPSSFLLKQIEFLKGQLPEAPVIGKQ
TPSLPPSLPGLRPREPVLLASSTRGRQVDIRGVPRGVHLRSQGLQRGFQHPSPRGRSLPQ
RGVDCLSSHFQELSIYQDQEQRILKFLEELGEGKATTAHDLSGKLGTPKKEINRVLYSLA
KKGKLQKEAGTPPLWKIAVSTQAWNQHSGVVRPDGHSQGAPNSDPSLEPEDRNSTSVSED
LLEPFIAVSAQAWNQHSGVVRPDSHSQGSPNSDPGLEPEDSNSTSALEDPLEFLDMAEIK
EKICDYLENVSDSSALNLAKNIGLTKARDINAVLIDMERQGDVYRQGTTPPIWHLTDKKR
ERMQIKRNTNSVPETAPAAIPETKRNAEFLTCNIPTSNASNNMVTTEKVENGQEPVIKLE
NRQEARPEPARLKPPVHYNGPSKAGYVDFENGQWATDDIPDDLNSIRAAPGEFRAIMEMP
SFYSHGLPRCSPYKKLTECQLKNPISGLLEYAQFASQTCEFNMIEQSGPPHEPRFKFQVV
INGREFPPAEAGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCVAVGAQTEPSV
SAPSKKVAKQMAAEEAMKALHGEATNSMASDNQPEGMISESIDNLESMMPNKVRKIGELV
RYLNTNPVGGLLEYARSHGFAAEFKLVDQSGPPHEPKFVYQAKVGGRWFPAVCAHSKKQG
KQEAADAALRVLIGENEKAERMGFTEVTPVTGASLRRIMLLLSRSPEAQPKTLPLTGSTE
HDQIAMLSHRCFNTLTNSFQPSLLGRKILAAIIMKKDSEDMGVVVSIGTGNRCVKGDSLS
LKGETVNDCHAEIISRRGFIRFLYSELMKYNSQTAKDSIFEPAKGGEKLQIKKTVSFHLY
ISTAPCGDGALFDKSCSDRAMESTESRHYPVFENPKQGKLRIKVENGEGTIPVESSDIVP
TWDGIRLGERLRTMSCSDKILRWNVLGLQGALLTHFLQPIYLKSVTLGYLFSQGHLTRAI
CCRVTRDGSAFEDGLRHPFIVNHPKVGRVSIYDSKRQSGKTKETSVNWCLADGYDLEILD
GTRGTVDGPRNELSRVSKKNIFLLFKKLCSFRYRRDLLRLSYGEAKKAARDYETAKNYFK
KGLKDMGYGNWISKPQEEKNFYLCPV

FIG. 23. Expression and Purification of recombinant human full-length ADAR1 in BJ2168 yeast expression system. (A) p424 10×His-tagged full-length ADAR1 vector map. (B) Schematic representation of Galactose-inducible expression system. (C) Coomassie Blue stain confirming Galactose-inducible expression of 10×His-tagged full-length ADAR1.

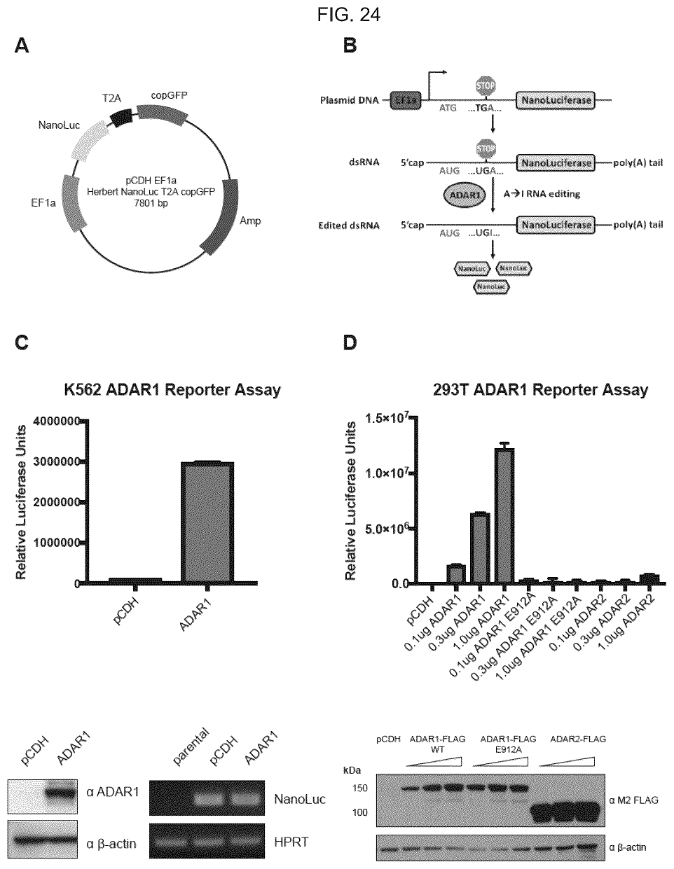

FIG. 24. Nano-luciferase-based RNA editase activity reporter assay in vitro. (A) Schematic representation of Nano-luciferase reporter design. Reporter was designed with a UGA stop codon between promotor and Nano-luciferase sequences (Herbert sequence). When there is no A-to-I editing in the cell, the stop codon in front of the Nano-luciferase sequence prevents its transcription. Therefore, there will be no signal. In the presence of ADAR1, the stop codon will be edited via ADAR1's A-to-I RNA editase activity and thereby no longer prevent the transcription of the Nano-luciferase sequence. Therefore, there will be a luminescence signal, which can be detected and quantified. (B) Lentiviral NanoLuciferase RNA editase reporter expression vector. (C) upper panel NanoLuciferase activity assay showing concentration-dependency and specificity for ADAR1 editase activity in HEK293T cells after co-transfection with FLAG-tagged ADAR1 constructs and NanoLuciferase reporter. lower panel α-FLAG Western Blot analysis demonstrating increasing FLAG-ADAR protein levels. (D) upper panel NanoLuciferase activity assay comparing ADAR1 RNA editase activity in K562 cells after co-transduction with pCDH/ADAR1 and NanoLuciferase reporter. lower panel α-ADAR1 Western Blot analysis demonstrating equal ADAR1 protein levels for all conditions (left) and RT-PCR showing equal expression of NanoLuciferase reporter for all conditions as well as in parental un-transduced K562 cells as a control (right).

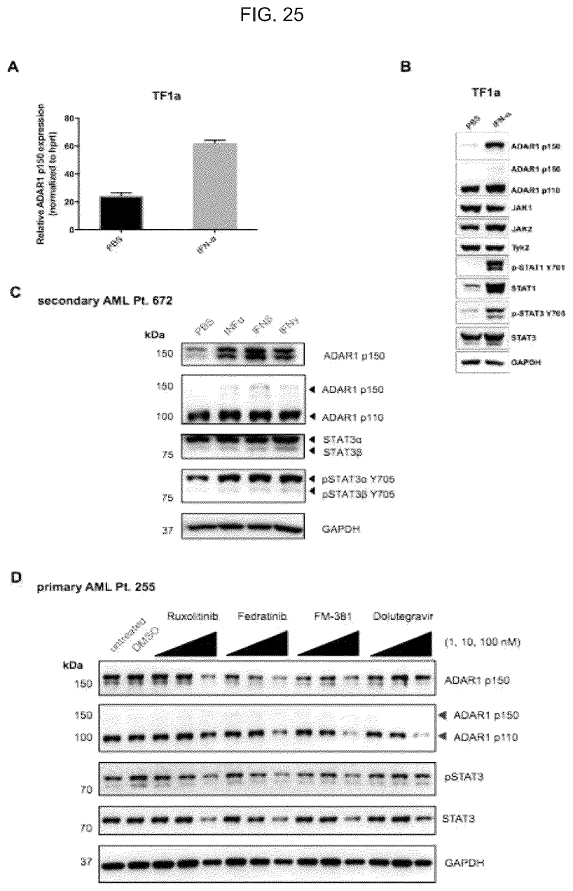

FIG. 25. Involvement of ADAR1 in the JAK/STAT pathway and JAK inhibitors as potential ADAR1-inhibiting agents. (A) ADAR1 p150 isoform expression level in TF1a cells as shown by qPCR 16 hrs after treatment with PBS (control) or Interferon alpha (normalized to HPRT). (B) Western Blot analysis of TF1a cells depicting protein levels of ADAR1 and various members of the JAK/STAT pathway 16 hrs after treatment with PBS (control) or Interferon alpha. (C) Western Blot analysis of secondary AML (patient 672) CD34+ cells showing protein levels of ADAR1, STAT3 and phospho-STAT3 Y705 16 hrs after treatment with PBS (control), interferon alpha, beta or gamma. (D) Western blot analysis of secondary AML (patient 255) CD34+ cells treated with FDA approved JAK2 inhibitors (ruxolitinib and fedratinib) compared with a JAK3 inhibitor (FM-381) at concentrations of 1 nM, 10 nM, and 100 nM.

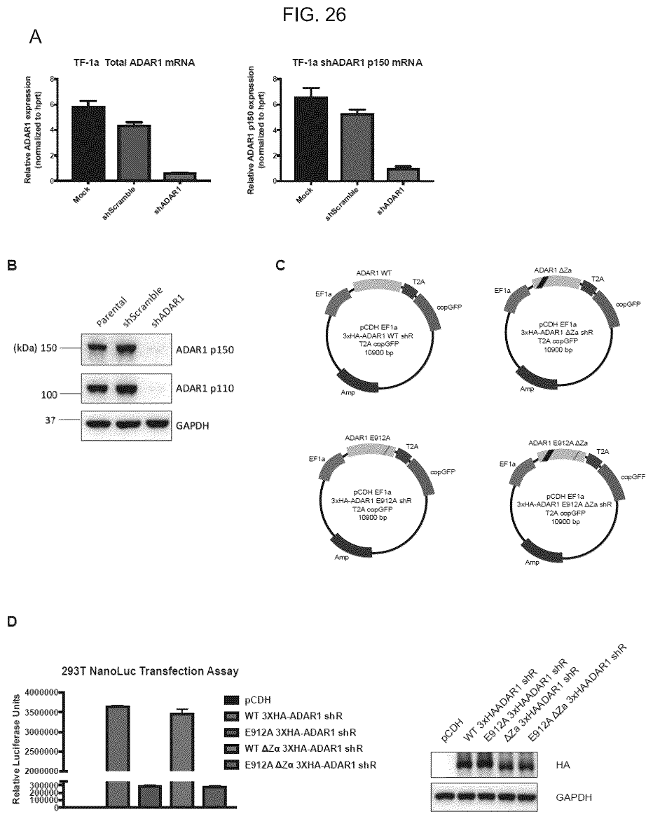

FIG. 26. Stable lentiviral shRNA-mediated knockdown of ADAR1 and stable lentiviral overexpression of ADAR1 wildtype and ADAR1 mutants after shADAR1 knockdown. (A) Total ADAR1 (left) and ADAR1 p150 isoform (right) expression levels in TF1a cells after transduction with shSchramble and shADAR1 as shown by qPCR (normalized to HPRT), confirming efficient (90%) shRNA-mediated knockdown of ADAR1. (B) Protein levels of ADAR1 in TF1a cells after transduction with shSchramble and shADAR1 as shown by Western Blot analysis, demonstrating efficient (90%) shRNA-mediated knockdown of ADAR1. (C) Lentiviral expression vectors of HA-tagged, shADAR1-resistant (shR) ADAR1 wildtype, ADAR1 editase-deficient mutant E921A, ADAR1 DNA-binding domain-deficient mutant dZa and ADAR1 mutant E912A dZa constructs. (D) NanoLuciferase activity assay comparing ADAR1 RNA editase activity in TF1a cells after co-transduction with pCDH/ADAR1 shR vectors and NanoLuciferase reporter into the background of shRNA-mediated ADAR1 knockdown (left). α-HA Western Blot analysis demonstrating similar ADAR1 protein levels for all conditions.

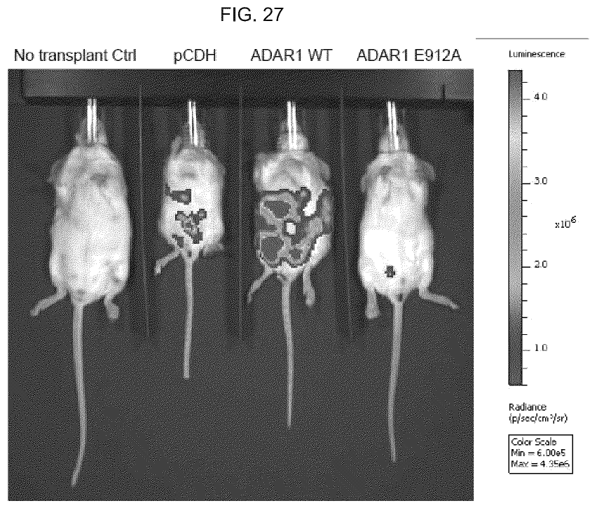

FIG. 27. Nano-luciferase-based RNA editase activity reporter assay in vivo. (A) IVIS® imaging of 6.5-week-old mice after neonatal intrahepatic transplantation with K562 cells co-transduced with pCDH/wildtype ADAR1/editase-deficient ADAR1 E912A and Nano-luciferase reporter demonstrating in vivo visualization of RNA editase activity.

### REFERENCES FOR EXAMPLE 1

1. Jamieson C H, et al. Granulocyte-macrophage progenitors as candidate leukemic stem cells in blast-crisis CML. N Engl J Med 2004; 351:657-67.
2. Rossi D J, Jamieson C H, Weissman I L. Stems cells and the pathways to aging and cancer. Cell 2008; 132:681-96.
3. Jamieson C H, Gotlib J, Durocher J A, et al. The JAK2 V617F mutation occurs in hematopoietic stem cells in polycythemia vera and predisposes toward erythroid differentiation. Proc Natl Acad Sci USA 2006; 103:6224-9.
4. Grinfeld J, Nangalia J, Baxter E J, et al. Classification and Personalized Prognosis in Myeloproliferative Neoplasms. N Engl J Med 2018; 379:1416-30.
5. Cross N C, Daley G Q, Green A R, et al. BCR-ABL1-positive CML and BCR-ABL1-negative chronic myeloproliferative disorders: some common and contrasting features. Leukemia 2008; 22:1975-89.
6. Asher S, McLornan D P, Harrison C N. Current and future therapies for myelofibrosis. Blood Rev 2020; 42:100715.
7. Bar-Natan M, Hoffman R. Developing strategies to reduce the duration of therapy for patients with myeloproliferative neoplasms. Expert Rev Hematol 2020; 13:1253-64.
8. Harrison C N, Schaap N, Vannucchi A M, et al. Janus kinase-2 inhibitor fedratinib in patients with myelofibrosis previously treated with ruxolitinib (JAKARTA-2): a single-arm, open-label, non-randomised, phase 2, multicentre study. Lancet Haematol 2017; 4:e317-e24.
9. Jamieson C, Hasserjian R, Gotlib J, et al. Effect of treatment with a JAK2-selective inhibitor, fedratinib, on bone marrow fibrosis in patients with myelofibrosis. J Transl Med 2015; 13:294.
10. Mascarenhas J, Hoffman R, Talpaz M, et al. Pacritinib vs Best Available Therapy, Including Ruxolitinib, in Patients With Myelofibrosis: A Randomized Clinical Trial. JAMA Oncol 2018; 4:652-9.
11. Mesa R A, Passamonti F. Individualizing Care for Patients With Myeloproliferative Neoplasms: Integrating Genetics, Evolving Therapies, and Patient-Specific Disease Burden. Am Soc Clin Oncol Educ Book 2016; 35:e324-35.
12. Cortes J. How to manage CML patients with comorbidities. Hematology Am Soc Hematol Educ Program 2020; 2020:237-42.
13. Gerds A T, Gotlib J, Bose P, et al. Myeloid/Lymphoid Neoplasms with Eosinophilia and TK Fusion Genes, Version 3.2021, NCCN Clinical Practice Guidelines in Oncology. J Natl Compr Cane Netw 2020; 18:1248-69.
14. Keller G, Brassat U, Braig M, Heim D, Wege H, Brummendorf T H. Telomeres and telomerase in chronic myeloid leukaemia: impact for pathogenesis, disease progression and targeted therapy. Hematol Oncol 2009; 27:123-9.
15. Artandi S E, DePinho R A. Telomeres and telomerase in cancer. Carcinogenesis 2010; 31:9-18.
16. Baerlocher G M, Burington B, Snyder D S. Telomerase Inhibitor Imetelstat in Essential Thrombocythemia and Myelofibrosis. N Engl J Med 2015; 373:2580.
17. Blackburn E H, Epel E S, Lin J. Human telomere biology: A contributory and interactive factor in aging, disease risks, and protection. Science 2015; 350:1193-8.
18. Braig M, et al. A ‘telomere-associated secretory phenotype’ cooperates with BCR-ABL to drive malignant proliferation of leukemic cells. Leukemia 2014; 28:2028-39.
19. Chiappori A A, et al. A randomized phase II study of the telomerase inhibitor imetelstat as maintenance therapy for advanced non-small-cell lung cancer. Ann Oncol 2015; 26:354-62.
20. Sharpless N E, DePinho R A. Telomeres, stem cells, senescence, and cancer. J Clin Invest 2004; 113:160-8.
21. Tefferi A, Lasho T L, Begna K H, et al. A Pilot Study of the Telomerase Inhibitor Imetelstat for Myelofibrosis. N Engl J Med 2015; 373:908-19.
22. Roake C M, Artandi S E. Regulation of human telomerase in homeostasis and disease. Nat Rev Mol Cell Biol 2020; 21:384-97.
23. Ruis P, et al. TRF2-independent chromosome end protection during pluripotency. Nature 2020.
24. Markiewicz-Potoczny M, Lobanova A, Loeb A M, et al. TRF2-mediated telomere protection is dispensable in pluripotent stem cells. Nature 2020.
25. Tan M H, Li Q, Shanmugam R, et al. Dynamic landscape and regulation of RNA editing in mammals. Nature 2017; 550:249-54.
26. Hartner J C, et al., ADAR1 is essential for the maintenance of hematopoiesis and suppression of interferon signaling. Nat Immunol 2009; 10:109-15.
27. Jiang Q, Isquith J, Zipeto M A, et al. Hyper-Editing of Cell-Cycle Regulatory and Tumor Suppressor RNA Promotes Malignant Progenitor Propagation. Cancer Cell 2019; 35:81-94 e7.
28. Hoffmeyer K, et al. Wnt/beta-catenin signaling regulates telomerase in stem cells and cancer cells. Science 2012; 336:1549-54.
29. Nusse R, Clevers H. Wnt/β-Catenin Signaling, Disease, and Emerging Therapeutic Modalities. Cell 2017; 169:985-99.
30. Lien W H, Fuchs E. Wnt some lose some: transcriptional governance of stem cells by Wnt/β-catenin signaling. Genes Dev 2014; 28:1517-32.
31. Park J I, Venteicher A S, Hong J Y, et al. Telomerase modulates Wnt signalling by association with target gene chromatin. Nature 2009; 460:66-72.
32. Zipeto M A, Court A C, Sadarangani A, et al. ADAR1 Activation Drives Leukemia Stem Cell Self-Renewal by Impairing Let-7 Biogenesis. Cell Stem Cell 2016; 19:177-91.
33. Bruedigam C, et al. Telomerase inhibition effectively targets mouse and human AML stem cells and delays relapse following chemotherapy. Cell Stem Cell 2014; 15:775-90.
34. Picardi E, et al., REDlportal: a comprehensive database of A-to-I RNA editing events in humans. Nucleic Acids Res 2017; 45:D750-D7.
35. Benjamini Y, et al., Controlling the false discovery rate in behavior genetics research. Behav Brain Res 2001; 125:279-84.
36. Hanzelmann et al., GSVA: gene set variation analysis for microarray and RNA-seq data. BMC Bioinformatics 2013; 14:7.
37. Zipeto M A, et al. ADAR1 Activation Drives Leukemia Stem Cell Self-Renewal by Impairing Let-7 Biogenesis. Cell Stem Cell 2016.
38. Petljak M, Alexandrov L B, Brammeld J S, et al. Characterizing Mutational Signatures in Human Cancer Cell Lines Reveals Episodic APOBEC Mutagenesis. Cell 2019; 176:1282-94 e20.
39. Schwarz M A, Lee D D, Bartlett S. Aminoacyl tRNA synthetase complex interacting multifunctional protein 1 simultaneously binds Glutamyl-Prolyl-tRNA synthetase and scaffold protein aminoacyl tRNA synthetase complex interacting multifunctional protein 3 of the multi-tRNA synthetase complex. Int J Biochem Cell Biol 2018; 99:197-202.
40. Sfeir A, et al., Loss of Rapl induces telomere recombination in the absence of NHEJ or a DNA damage signal. Science 2010; 327:1657-61.

### SUPPLEMENTAL REFERENCES

1. Genomes Project C, Auton A, Brooks L D, et al. A global reference for human genetic variation. Nature 2015; 526:68-74.
2. Li H, Durbin R. Fast and accurate short read alignment with Burrows-Wheeler transform. Bioinformatics 2009; 25:1754-60.
3. Li H, Handsaker B, Wysoker A, et al. The Sequence Alignment/Map format and SAMtools. Bioinformatics 2009; 25:2078-9.
4. McKenna A, Hanna M, Banks E, et al. The Genome Analysis Toolkit: a MapReduce framework for analyzing next-generation DNA sequencing data. Genome Res 2010; 20:1297-303.
5. Cibulskis K, Lawrence M S, Carter S L, et al. Sensitive detection of somatic point mutations in impure and heterogeneous cancer samples. Nat Biotechnol 2013; 31:213-9.
6. Lai Z, Markovets A, Ahdesmaki M, et al. VarDict: a novel and versatile variant caller for next-generation sequencing in cancer research. Nucleic Acids Res 2016; 44:e108.
7. Picardi E, D'Erchia A M, Lo Giudice C, Pesole G. REDlportal: a comprehensive database of A-to-I RNA editing events in humans. Nucleic Acids Res 2017; 45:D750-D7.
8. Ramaswami G, Li J B. RADAR: a rigorously annotated database of A-to-I RNA editing. Nucleic Acids Res 2014; 42:D109-13.
9. Kiran A, Baranov P V. DARNED: a DAtabase of RNa EDiting in humans. Bioinformatics 2010; 26:1772-6.
10. Heldenbrand J R, Baheti S, Bockol M A, et al. Recommendations for performance optimizations when using GATK3.8 and GATK4. BMC Bioinformatics 2019; 20:557.
11. Kim S, Scheffler K, Halpern A L, et al. Strelka2: fast and accurate calling of germline and somatic variants. Nat Methods 2018; 15:591-4.
12. Koboldt D C, Zhang Q, Larson D E, et al. VarScan 2: somatic mutation and copy number alteration discovery in cancer by exome sequencing. Genome Res 2012; 22:568-76.
13. Fan Y, et al. Genome Biol 2016; 17:178.
14. Consortium ITP-CAoWG. Pan-cancer analysis of whole genomes. Nature 2020; 578:82-93.
15. Lek M, Karczewski K J, Minikel E V, et al. Analysis of protein-coding genetic variation in 60,706 humans. Nature 2016; 536:285-91.
16. Ramaswami G, Lin W, Piskol R, Tan M H, Davis C, Li J B. Accurate identification of human Alu and non-Alu RNA editing sites. Nat Methods 2012; 9:579-81.
17. Ramaswami G, Zhang R, Piskol R, et al. Identifying RNA editing sites using RNA sequencing data alone. Nat Methods 2013; 10:128-32.
18. Piskol R, Ramaswami G, Li J B. Reliable identification of genomic variants from RNA-seq data. Am J Hum Genet 2013; 93:641-51.
19. Dobin A, Davis C A, Schlesinger F, et al. STAR: ultrafast universal RNA-seq aligner. Bioinformatics 2013; 29:15-21.
20. Li B, Dewey C N. RSEM: accurate transcript quantification from RNA-Seq data with or without a reference genome. BMC Bioinformatics 2011; 12:323.
21. Robinson M D, McCarthy D J, Smyth G K. edgeR: a Bioconductor package for differential expression analysis of digital gene expression data. Bioinformatics 2010; 26:139-40.
22. Ritchie M E, Phipson B, Wu D, et al. limma powers differential expression analyses for RNA-sequencing and microarray studies. Nucleic Acids Res 2015; 43:e47.
23. Law C W, Chen Y, Shi W, Smyth G K. voom: Precision weights unlock linear model analysis tools for RNA-seq read counts. Genome Biol 2014; 15:R29.
24. Benjamini Y, Drai D, Elmer G, Kafkafi N, Golani I. Controlling the false discovery rate in behavior genetics research. Behav Brain Res 2001; 125:279-84.
25. Hanzelmann S, Castelo R, Guinney J. GSVA: gene set variation analysis for microarray and RNA-seq data. BMC Bioinformatics 2013; 14:7.
26. Jamieson C H, Ailles L E, Dylla S J, et al. Granulocyte-macrophage progenitors as candidate leukemic stem cells in blast-crisis CML. N Engl J Med 2004; 351:657-67.
27. Zipeto M A, Court A C, Sadarangani A, et al. ADAR1 Activation Drives Leukemia Stem Cell Self-Renewal by Impairing Let-7 Biogenesis. Cell Stem Cell 2016.
28. Jiang Q, Isquith J, Zipeto M A, et al. Hyper-Editing of Cell-Cycle Regulatory and Tumor Suppressor RNA Promotes Malignant Progenitor Propagation. Cancer Cell 2019; 35:81-94 e7.
29. Abrahamsson A E, Geron I, Gotlib J, et al. Glycogen synthase kinase 3beta missplicing contributes to leukemia stem cell generation. Proc Natl Acad Sci USA 2009; 106:3925-9.
30. Goff D J, Recart A C, Sadarangani A, et al. A Pan-BCL2 inhibitor renders bone-marrow-resident human leukemia stem cells sensitive to tyrosine kinase inhibition. Cell Stem Cell 2013; 12:316-28.
31. Jiang Q, Crews L A, Jamieson C H. ADAR1 promotes malignant progenitor reprogramming in chronic myeloid leukemia. Proc Natl Acad Sci USA 2013; 110:1041-6.
32. Zipeto M A, Court A C, Sadarangani A, et al. ADAR1 Activation Drives Leukemia Stem Cell Self-Renewal by Impairing Let-7 Biogenesis. Cell Stem Cell 2016; 19:177-91.
33. Crews L A, Balaian L, Delos Santos N P, et al. RNA Splicing Modulation Selectively Impairs Leukemia Stem Cell Maintenance in Secondary Human AML. Cell Stem Cell 2016; 19:599-612.
34. Goff D J, Court Recart A, Sadarangani A, et al. A Pan-BCL2 inhibitor renders bone-marrow-resident human leukemia stem cells sensitive to tyrosine kinase inhibition. Cell Stem Cell 2013; 12:316-28.

### REFERENCES EXAMPLE 2

Abrahamsson, A. E., Geron, I., Gotlib, J., Dao, K. H., Barroga, C. F., Newton, LG., Giles, F. J., Durocher, J., Creusot, R. S., Karimi, M., et al., 2009. Glycogen synthase kinase 3beta missplicing contributes to leukemia stem cell generation. Proc. Natl. Acad. Sci. USA 106, 3925-3929.
Alexandrov, L. B., Nik-Zainal, S., Wedge, D. C., Aparicio, S. A., Behjati, S., Biankin, A. V., Bignell, G. R., Bolli, N., Borg, A., Borresen-Dale, A. L., et al., Australian Pancreatic Cancer Genome Initiative; ICGC Breast Cancer Consortium; ICGC MMML-Seq Consortium; ICGC PedBrain, 2013a. Signatures of mutational processes in human cancer. Nature 500, 415-421.
Alexandrov, L. B., Nik-Zainal, S., Wedge, D. C., Campbell, P. J., Stratton, M. R., 2013b. Deciphering signatures of mutational processes operative in human cancer. Cell Rep. 3, 246-259.
Alexandrov, L. B., Kim, J., Haradhvala, N. J., Huang, M. N., Tian Ng, A. W., Wu, Y., Boot, A., Covington, K. R., Gordenin, D. A., Bergstrom, E. N., et al., PCAWG Mutational Signatures Working Group; PCAWG Consortium, 2020. The repertoire of mutational signatures in human cancer. Nature 578, 94-101.
Auton, A., Brooks, L. D., Durbin, R. M., Garrison, E. P., Kang, H. M., Korbel, J. O., Marchini, J. L., McCarthy, S., McVean, G. A., Abecasis, G. R., 1000 Genomes Project Consortium, 2015. A global reference for human genetic variation. Nature 526, 68-74. Bao, E. L., Nandakumar, S. K., Liao, X., Bick, A. G., Karjalainen, J., Tabaka, M., Gan, O. I., Havulinna, A. S., Kiiskinen, T. T. J., Lareau, C. A., et al., FinnGen; 23andMe Research Team, 2020. Inherited myeloproliferative neoplasm risk affects haematopoietic stem cells. Nature 586, 769-775.
Benjamin, D. S. T., Cibulskis, K., Getz, G., Stewart, C., Lichtenstein, L., 2019. Calling Somatic SNVs and Indels with Mutect2. bioRxiv. doi:10.1101/861054.
Benjamini, Y., Drai, D., Elmer, G., Kafkafi, N., Golani, I., 2001. Controlling the false discovery rate in behavior genetics research. Behay. Brain Res. 125, 279-284.
Bergstrom, E. N., Huang, M. N., Mahto, U., Barnes, M., Stratton, M. R., Rozen, S. G., Alexandrov, L. B., 2019. SigProfilerMatrixGenerator: a tool for visualizing and exploring patterns of small mutational events. BMC Genomics 20, 685.
Bowman, T., Broome, M. A., Sinibaldi, D., Wharton, W., Pledger, W. J., Sedivy, J. M., Irby, R., Yeatman, T., Courtneidge, S. A., Jove, R., 2001. Stat3-mediated Myc expression is required for Src transformation and PDGF-induced mitogenesis. Proc. Natl. Acad. Sci. USA 98, 7319-7324.
Buisson, R., Langenbucher, A., Bowen, D., Kwan, E. E., Benes, C. H., Zou, L., Lawrence, M. S., 2019. Passenger hotspot mutations in cancer driven by APOBEC3A and mesoscale genomic features. Science 364, eaaw2872.
Burns, M. B., Lackey, L., Carpenter, M. A., Rathore, A., Land, A. M., Leonard, B., Refsland, E. W., Kotandeniya, D., Tretyakova, N., Nikas, J. B., et al., 2013a. APOBEC3B is an enzymatic source of mutation in breast cancer. Nature 494, 366-370.
Burns, M. B., Temiz, N. A., Harris, R. S., 2013b. Evidence for APOBEC3B mutagenesis in multiple human cancers. Nat. Genet. 45, 977-983.
Chen, L., Li, Y., Lin, C. H., Chan, T. H., Chow, R. K., Song, Y., Liu, M., Yuan, Y. F., Fu, L., Kong, K. L., et al., 2013. Recoding RNA editing of AZIN1 predisposes to hepatocellular carcinoma. Nat. Med. 19, 209-216.
Chen, X., Schulz-Trieglaff, O., Shaw, R., Barnes, B., Schlesinger, F., Milberg, M., Cox, A. J., Kruglyak, S., Saunders, C. T., 2016. Manta: rapid detection of structural variants and indels for germline and cancer sequencing applications. Bioinformatics 32, 1220-1222.
Chua, B. A., Van Der Werf, I., Jamieson, C., Signer, R. A. J., 2020. Post-Transcriptional Regulation of Homeostatic, Stressed, and Malignant Stem Cells. Cell Stem Cell 26, 138-159.
Cibulskis, K., Lawrence, M. S., Carter, S. L., Sivachenko, A., Jaffe, D., Sougnez, C., Gabriel, S., Meyerson, M., Lander, E. S., Getz, G., 2013. Sensitive detection of somatic point mutations in impure and heterogeneous cancer samples. Nat. Biotechnol. 31, 213-219.
Cowen, L., Ideker, T., Raphael, B. J., Sharan, R., 2017. Network propagation: a universal amplifier of genetic associations. Nat. Rev. Genet. 18, 551-562.
Danecek, P., Auton, A., Abecasis, G., Albers, C. A., Banks, E., DePristo, M. A., Handsaker, R. E., Lunter, G., Marth, G. T., Sherry, S. T., et al., 1000 Genomes Project Analysis Group, 2011. The variant call format and VCFtools. Bioinformatics 27, 2156-2158.
Di Giorgio, S., Martignano, F., Torcia, M. G., Mattiuz, G., Conticello, S. G., 2020. Evidence for host-dependent RNA editing in the transcriptome of SARS-CoV-2. Sci. Adv. 6, eabb5813.
Dobin, A., Davis, C. A., Schlesinger, F., Drenkow, J., Zaleski, C., Jha, S., Batut, P., Chaisson, M., Gingeras, T. R., 2013. STAR: ultrafast universal RNA-seq aligner. Bioinformatics 29, 15-21.
Dong, X., Chen, G., Cai, Z., Li, Z., Qiu, L., Xu, H., Yuan, Y., Liu, X. L., Liu, J., 2018. CDK13 RNA Over-Editing Mediated by ADAR1 Associates with Poor Prognosis of Hepatocellular Carcinoma Patients. Cell. Physiol. Biochem. 47, 2602-2612.
Eide, C. A., Druker, B. J., 2017. Understanding cancer from the stem cells up. Nat. Med. 23, 656-657.
Fan, Y., Xi, L., Hughes, D. S., Zhang, J., Zhang, J., Futreal, P. A., Wheeler, D. A., Wang, W., 2016. MuSE: accounting for tumor heterogeneity using a sample-specific error model improves sensitivity and specificity in mutation calling from sequencing data. Genome Biol. 17, 178.
Faust, G. G., Hall, I. M., 2014. SAMBLASTER: fast duplicate marking and structural variant read extraction. Bioinformatics 30, 2503-2505.
Geoffroy, V., Herenger, Y., Kress, A., Stoetzel, C., Piton, A., Dollfus, H., Muller, J., 2018. AnnotSV: an integrated tool for structural variations annotation. Bioinformatics 34, 3572-3574.
Gishizky, M. L., Johnson-White, J., Witte, O. N., 1993. Efficient transplantation of BCR-ABL-induced chronic myelogenous leukemia-like syndrome in mice. Proc. Natl. Acad. Sci. USA 90, 3755-3759.
Goff, D. J., Court Recart, A., Sadarangani, A., Chun, H. J., Barrett, C. L., Krajewska, M., Leu, H., Low-Marchelli, J., Ma, W., Shih, A. Y., et al., 2013. A Pan-BCL2 inhibitor renders bone-marrow-resident human leukemia stem cells sensitive to tyrosine kinase inhibition. Cell Stem Cell 12, 316-328.
Goldberg, L., Abutbul-Amitai, M., Paret, G., Nevo-Caspi, Y., 2017. Alternative Splicing of STAT3 Is Affected by RNA Editing. DNA Cell Biol. 36, 367-376.
Grinfeld, J., Nangalia, J., Baxter, E. J., Wedge, D. C., Angelopoulos, N., Cantrill, R., Godfrey, A. L., Papaemmanuil, E., Gundem, G., MacLean, C., et al., 2018. Classification and Personalized Prognosis in Myeloproliferative Neoplasms. N. Engl. J. Med. 379, 1416-1430.
Grunewald, J., Zhou, R., Garcia, S. P., Iyer, S., Lareau, C. A., Aryee, M. J., Joung, J. K., 2019. Transcriptome-wide off-target RNA editing induced by CRISPR-guided DNA base. Nature 569, 433-437.
Gu, Z., Gu, L., Eils, R., Schlesner, M., Brors, B., 2014. circlize Implements and enhances circular visualization in R. Bioinformatics 30, 2811-2812.
Guallar, D., Fuentes-Iglesias, A., Souto, Y., Ameneiro, C., Freire-Agulleiro, O., Pardavila, J. A., Escudero, A., Garcia-Outeiral, V., Moreira, T., Saenz, C., et al., 2020. ADAR1-Dependent RNA Editing Promotes MET and iPSC Reprogramming by Alleviating ER Stress. Cell Stem Cell 27, 300-314.e11.
Han, L., Diao, L., Yu, S., Xu, X., Li, J., Zhang, R., Yang, Y., Werner, H. M. J., Eterovic, A. K., Yuan, Y., et al., 2015. The Genomic Landscape and Clinical Relevance of A-to-I RNA Editing in Human Cancers. Cancer Cell 28, 515-528.
Hanzelmann, S., Castelo, R., Guinney, J., 2013. GSVA: gene set variation analysis for microarray and RNA-seq data. BMC Bioinformatics 14, 7.
Hartner, J. C., Walkley, C. R., Lu, J., Orkin, S. H., 2009. ADAR1 is essential for the maintenance of hematopoiesis and suppression of interferon signaling. Nat. Immunol. 10, 109-115.
Heldenbrand, J. R., Baheti, S., Bockol, M. A., Drucker, T. M., Hart, S. N., Hudson, M. E.,
Iyer, R. K., Kalmbach, M. T., Kendig, K. I., Klee, E. W., et al., 2019. Recommendations for performance optimizations when using GATK3.8 and GATK4. BMC Bioinformatics 20, 557.
Hirai, H., Karian, P., Kikyo, N., 2011. Regulation of embryonic stem cell self-renewal and pluripotency by leukaemia inhibitory factor. Biochem. J. 438, 11-23.
ICGC/TCGA Pan-Cancer Analysis of Whole Genomes Consortium, 2020. Pan-cancer analysis of whole genomes. Nature 578, 82-93.
Iniguez, A. B., Stolte, B., Wang, E. J., Conway, A. S., Alexe, G., Dharia, N. V., Kwiatkowski, N., Zhang, T., Abraham, B. J., Mora, J., et al., 2018. EWS/FLI Confers Tumor Cell Synthetic Lethality to CDK12 Inhibition in Ewing Sarcoma. Cancer Cell 33, 202-216.e6.
Ishizuka, J. J., Manguso, R. T., Cheruiyot, C. K., Bi, K., Panda, A., Iracheta-Vellve, A., Miller, B. C., Du, P. P., Yates, K. B., Dubrot, J., et al., 2019. Loss of ADAR1 in tumours overcomes resistance to immune checkpoint blockade. Nature 565, 43-48.
Jager, R., Gisslinger, H., Fuchs, E., Bogner, E., Milosevic Feenstra, J. D., Weinzierl, J., Schischlik, F., Gisslinger, B., Schalling, M., Zorer, M., et al., 2020. Germline Genetic Factors Influence Outcome of Interferon Alpha Therapy in Polycythemia Vera. Blood Published online Aug. 19, 2020 doi:10.1182/blood.2020005792.
Jamieson, C. H., Ailles, L. E., Dylla, S. J., Muijtj ens, M., Jones, C., Zehnder, J. L., Gotlib, J., Li, K., Manz, M. G., Keating, A., et al., 2004. Granulocyte-macrophage progenitors as candidate leukemic stem cells in blast-crisis CML. N. Engl. J. Med. 351, 657-667.
Jeffares, D. C., Jolly, C., Hoti, M., Speed, D., Shaw, L., Rallis, C., Balloux, F., Dessimoz, C., Bähler, J., Sedlazeck, F. J., 2017. Transient structural variations have strong effects on quantitative traits and reproductive isolation in fission yeast. Nat. Commun. 8, 14061.
Jiang, Q., Crews, L. A., Barrett, C. L., Chun, H. J., Court, A. C., Isquith, J. M., Zipeto, M. A., Goff, D. J., Minden, M., Sadarangani, A., et al., 2013. ADAR1 promotes malignant progenitor reprogramming in chronic myeloid leukemia. Proc. Natl. Acad. Sci. USA 110, 1041-1046.
Jiang, Q., Crews, L. A., Holm, F., Jamieson, C. H. M., 2017. RNA editing-dependent epitranscriptome diversity in cancer stem cells. Nat. Rev. Cancer 17, 381-392.
Jiang, Q., Isquith, J., Zipeto, M. A., Diep, R. H., Pham, J., Delos Santos, N., Reynoso, E., Chau, J., Leu, H., Lazzari, E., et al., 2019. Hyper-Editing of Cell-Cycle Regulatory and Tumor Suppressor RNA Promotes Malignant Progenitor Propagation. Cancer Cell 35, 81-94. e7.
Kapoor, U., Licht, K., Amman, F., Jakobi, T., Martin, D., Dieterich, C., Jantsch, M. F., 2020. ADAR-deficiency perturbs the global splicing landscape in mouse tissues. Genome Res. 30, 1107-1118.
Kim, S., Scheffler, K., Halpern, A. L., Bekritsky, M. A., Noh, E., Kallberg, M., Chen, X., Kim, Y., Beyter, D., Krusche, P., Saunders, C. T., 2018. Strelka2: fast and accurate calling of germline and somatic variants. Nat. Methods 15, 591-594.
Kiran, A., Baranov, P. V., 2010. DARNED: a DAtabase of RNa EDiting in humans. Bioinformatics 26, 1772-1776.
Kleppe, M., Koche, R., Zou, L., van Galen, P., Hill, C. E., Dong, L., De Groote, S., Papalexi, E., Hanasoge Somasundara, A. V., Cordner, K., et al., 2018. Dual Targeting of Oncogenic Activation and Inflammatory Signaling Increases Therapeutic Efficacy in Myeloproliferative Neoplasms. Cancer Cell 33, 29-43.e7.
Koboldt, D. C., Zhang, Q., Larson, D. E., Shen, D., McLellan, M. D., Lin, L., Miller, C. A., Mardis, E. R., Ding, L., Wilson, R. K., 2012. VarScan 2: somatic mutation and copy number alteration discovery in cancer by exome sequencing. Genome Res. 22, 568-576.
Komor, A. C., Kim, Y. B., Packer, M. S., Zuris, J. A., Liu, D. R., 2016. Programmable editing of a target base in genomic DNA without double-stranded DNA cleavage. Nature 533, 420-424.
Lai, Z., Markovets, A., Ahdesmaki, M., Chapman, B., Hofmann, O., McEwen, R., Johnson, J., Dougherty, B., Barrett, J. C., Dry, J. R., 2016. VarDict: a novel and versatile variant caller for next-generation sequencing in cancer research. Nucleic Acids Res. 44, e108.
Law, C. W., Chen, Y., Shi, W., Smyth, G. K., 2014. voom: Precision weights unlock linear model analysis tools for RNA-seq read counts. Genome Biol. 15, R29.
Layer, R. M., Chiang, C., Quinlan, A. R., Hall, I. M., 2014. LUMPY: a probabilistic framework for structural variant discovery. Genome Biol. 15, R84.
Lazzari, E., Mondala, P. K., Santos, N. D., Miller, A. C., Pineda, G., Jiang, Q., Leu, H., Ali, S. A., Ganesan, A. P., Wu, C. N., et al., 2017. Alu-dependent RNA editing of GLI1 promotes malignant regeneration in multiple myeloma. Nat. Commun. 8, 1922.
Lek, M., Karczewski, K. J., Minikel, E. V., Samocha, K. E., Banks, E., Fennell, T., O'Donnell-Luria, A. H., Ware, J. S., Hill, A. J., Cummings, B. B., et al., Exome Aggregation Consortium, 2016. Analysis of protein-coding genetic variation in 60,706 humans. Nature 536, 285-291.
Li, H., 2013. Aligning sequence reads, clone sequences and assembly contigs with BWA-MEM. arXiv 1303.3997.
Li, B., Dewey, C. N., 2011. RSEM: accurate transcript quantification from RNA-Seq data with or without a reference genome. BMC Bioinformatics 12, 323.
Li, H., Durbin, R., 2009. Fast and accurate short read alignment with Burrows-Wheeler transform. Bioinformatics 25, 1754-1760.
Li, H., Handsaker, B., Wysoker, A., Fennell, T., Ruan, J., Homer, N., Marth, G., Abecasis, G., Durbin, R., 1000 Genome Project Data Processing Subgroup, 2009. The Sequence Alignment/Map format and SAMtools. Bioinformatics 25, 2078-2079.
Lin, J. H., 1991. Divergence Measures Based on the Shannon Entropy. IEEE Transactions on Information Theory 37, 145-151.
Mannion, N. M., Greenwood, S. M., Young, R., Cox, S., Brindle, J., Read, D., Nellaker, C., Vesely, C., Ponting, C. P., McLaughlin, P. J., et al., 2014. The RNA-editing enzyme ADAR1 controls innate immune responses to RNA. Cell Rep. 9, 1482-1494.
Mansi, L., Tangaro, M. A., Lo Giudice, C., Flati, T., Kopel, E., Schaffer, A. A., Castrignano, T., Chillemi, G., Pesole, G., Picardi, E., 2021. REDIportal: millions of novel A-to-I RNA editing events from thousands of RNAseq experiments. Nucleic acids research 49, D1012-D1019.
Martin, M., 2011. Cutadapt Removes Adapter Sequences From High-Throughput Sequencing Reads. EMBnet.journal.doi:10.14806/ej.17.1.200.
McKenna, A., Hanna, M., Banks, E., Sivachenko, A., Cibulskis, K., Kernytsky, A., Garimella, K., Altshuler, D., Gabriel, S., Daly, M., DePristo, M. A., 2010. The Genome Analysis Toolkit: a MapReduce framework for analyzing next-generation DNA sequencing data. Genome Res. 20, 1297-1303.
Meisel, M., et al., 2018. Microbial signals drive pre-leukaemic myeloproliferation in a Tet2-deficient host. Nature 557, 580-584.
Mesa, R. A., Jamieson, C., Bhatia, R., Deininger, M. W., Fletcher, C. D., Gerds, A. T., Gojo, I., Gotlib, J., Gundabolu, K., Hobbs, G., et al., 2017. NCCN Guidelines Insights: Myeloproliferative Neoplasms, Version 2.2018. J. Natl. Compr. Canc. Netw. 15, 1193-1207.
Miles, L. A., et al., 2020. Single-cell mutation analysis of clonal evolution in myeloid malignancies. Nature 587, 477-482.
Nusse, R., Clevers, H., 2017. Wnt/β-Catenin Signaling, Disease, and Emerging Therapeutic Modalities. Cell 169, 985-999.
Obenchain, V., et al., 2014. Variant Annotation: a Bioconductor package for exploration and annotation of genetic variants. Bioinformatics 30, 2076-2078.
Pardanani, A., Finke, C., Abdelrahman, R. A., Lasho, T. L., Tefferi, A., 2013. Associations and prognostic interactions between circulating levels of hepcidin, ferritin and inflammatory cytokines in primary myelofibrosis. Am. J. Hematol. 88, 312-316.
Peng, X., Xu, X., Wang, Y., Hawke, D. H., Yu, S., Han, L., Zhou, Z., Mojumdar, K., Jeong, K. J., Labrie, M., et al., 2018. A-to-I RNA Editing Contributes to Proteomic Diversity in Cancer. Cancer Cell 33, 817-828.e7.
Petljak, M., Alexandrov, L. B., Brammeld, J. S., Price, S., Wedge, D. C., Grossmann, S., Dawson, K. J., Ju, Y. S., Iorio, F., Tubio, J. M. C., et al., 2019. Characterizing Mutational Signatures in Human Cancer Cell Lines Reveals Episodic APOBEC Mutagenesis. Cell 176, 1282-1294.e20.
Picardi, E., Pesole, G., 2013. REDItools: high-throughput RNA editing detection made easy. Bioinformatics 29, 1813-1814.
Piskol, R., Ramaswami, G., Li, J. B., 2013. Reliable identification of genomic variants from RNA-seq data. Am. J. Hum. Genet. 93, 641-651.
Quinlan, A. R., Hall, I. M., 2010. BEDTools: a flexible suite of utilities for comparing genomic features. Bioinformatics 26, 841-842.
Ramaswami, G., Li, J. B., 2014. RADAR: a rigorously annotated database of A-to-I RNA editing. Nucleic Acids Res. 42, D109-D113.
Ramaswami, G., Lin, W., Piskol, R., Tan, M. H., Davis, C., Li, J. B., 2012. Accurate identification of human Alu and non-Alu RNA editing sites. Nat. Methods 9, 579-581.
Ramaswami, G., Zhang, R., Piskol, R., Keegan, L. P., Deng, P., O'Connell, M. A., Li, J. B., 2013. Identifying RNA editing sites using RNA sequencing data alone. Nat. Methods 10, 128-132.
Ramos, A. H., Lichtenstein, L., Gupta, M., Lawrence, M. S., Pugh, T. J., Saksena, G., Meyerson, M., Getz, G., 2015. Oncotator: cancer variant annotation tool. Hum. Mutat. 36, E2423-E2429.
Reimand, J., Kull, M., Peterson, H., Hansen, J., Vilo, J., 2007. g:Profiler—a web-based toolset for functional profiling of gene lists from large-scale experiments. Nucleic Acids Res. 35, W193-W200.
Ritchie, M. E., et al., 2015. limma powers differential expression analyses for RNA-sequencing and microarray studies. Nucleic Acids Res. 43, e47.
Robinson, M. D., McCarthy, D. J., Smyth, G. K., 2010. edgeR: a Bioconductor package for differential expression analysis of digital gene expression data. Bioinformatics 26, 139-140.
Rosenthal, S. B., Len, J., Webster, M., Gary, A., Birmingham, A., Fisch, K. M., 2018. Interactive network visualization in Jupyter notebooks: visJS2jupyter. Bioinformatics 34, 126-128.
Rossi, D. J., Jamieson, C. H., Weissman, I. L., 2008. Stems cells and the pathways to aging and cancer. Cell 132, 681-696.
Shannon, P., Markiel, A., Ozier, O., Baliga, N. S., Wang, J. T., Ramage, D., Amin, N., Schwikowski, B., Ideker, T., 2003. Cytoscape: a software environment for integrated models of biomolecular interaction networks. Genome Res. 13, 2498-2504.
Sharma, S., Patnaik, S. K., Taggart, R. T., Kannisto, E. D., Enriquez, S. M., Gollnick, P., Baysal, B. E., 2015. APOBEC3A cytidine deaminase induces RNA editing in monocytes and macrophages. Nat. Commun. 6, 6881.
Shlush, L. I., Zandi, S., Mitchell, A., Chen, W. C., Brandwein, J. M., Gupta, V., Kennedy, J. A., Schimmer, A. D., Schuh, A. C., Yee, K. W., et al., HALT Pan-Leukemia Gene Panel Consortium, 2014. Identification of leukaemic haematopoietic stem cells in acute leukaemia. Nature 506, 328-333.
Signer, R. A., Magee, J. A., Salic, A., Morrison, S. J., 2014. Haematopoietic stem cells require a highly regulated protein synthesis rate. Nature 509, 49-54.
Solomon, O., Di Segni, A., Cesarkas, K., Porath, H. T., Marcu-Malina, V., Mizrahi, O., Stern-Ginossar, N., Kol, N., Farage-Barhom, S., Glick-Saar, E., et al., 2017. RNA editing by ADAR1 leads to context-dependent transcriptome-wide changes in RNA secondary structure. Nat. Commun. 8, 1440.
Sukhai, M. A, et al., 2019. Somatic Tumor Variant Filtration Strategies to Optimize Tumor-Only Molecular Profiling Using Targeted Next-Generation Sequencing Panels. J. Mol. Diagn. 21, 261-273.
Szklarczyk, D., Franceschini, A., Wyder, S., Forslund, K., Heller, D., Huerta-Cepas, J., Simonovic, M., Roth, A., Santos, A., Tsafou, K. P., et al., 2015. STRING v10: protein-protein interaction networks, integrated over the tree of life. Nucleic Acids Res. 43, D447-D452.
Szklarczyk, D., Morris, J. H., Cook, H., Kuhn, M., Wyder, S., Simonovic, M., Santos, A., Doncheva, N. T., Roth, A., Bork, P., et al., 2017. The STRING database in 2017: quality-controlled protein-protein association networks, made broadly accessible. Nucleic Acids Res. 45 (D1), D362-D368.
Talevich, E., Shain, A. H., Botton, T., Bastian, B. C., 2016. CNVkit: Genome-Wide Copy Number Detection and Visualization from Targeted DNA Sequencing. PLoS Comput. Biol. 12, e1004873.
Tan, M. H., et al. Nature 550, 249-254.
Tarasov, A., Vilella, A. J., Cuppen, E., Nijman, I. J., Prins, P., 2015. Sambamba: fast processing of NGS alignment formats. Bioinformatics 31, 2032-2034.
Tarca, A. L., Draghici, S., Khatri, P., Hassan, S. S., Mittal, P., Kim, J. S., Kim, C. J., Kusanovic, J. P., Romero, R., 2009. A novel signaling pathway impact analysis. Bioinformatics 25, 75-82.
Tefferi, A., Vaidya, R., Caramazza, D., Finke, C., Lasho, T., Pardanani, A., 2011. Circulating interleukin (IL)-8, IL-2R, IL-12, and IL-15 levels are independently prognostic in primary myelofibrosis: a comprehensive cytokineprofiling study. J. Clin. Oncol. 29, 1356-1363.
Tefferi, A., Guglielmelli, P., Lasho, T. L., Gangat, N., Ketterling, R. P., Pardanani, A., Vannucchi, A. M., 2018. MIPSS70+ Version 2.0: Mutation and Karyotype-Enhanced International Prognostic Scoring System for Primary Myelofibrosis. J. Clin. Oncol. 36, 1769-1770.
van der Maaten, L., Hinton, G., 2008. Visualizing Data using t-SNE. J. Mach. Learn. Res. 9, 2579-2605.
Vannucchi, A. M., Verstovsek, S., Guglielmelli, P., Griesshammer, M., Burn, T. C., Naim, A., Paranagama, D., Marker, M., Gadbaw, B., Kiladjian, J. J., 2017. Ruxolitinib reduces JAK2 p.V617F allele burden in patients with polycythemia vera enrolled in the RESPONSE study. Ann. Hematol. 96, 1113-1120
Verstovsek, S., et al., 2012. A double-blind, placebo-controlled trial of ruxolitinib for myelofibrosis. N. Engl. J. Med. 366, 799-807.
Verstovsek, S., et al., 2017. Long-term survival in patients treated with ruxolitinib for myelofibrosis: COMFORT-I and -II pooled analyses. J. Hematol. Oncol. 10, 156.
Wang, K., Li, M., Hakonarson, H., 2010. ANNOVAR: functional annotation of genetic variants from high-throughput sequencing data. Nucleic Acids Res. 38, e164.
Wu, J., Keng, et al., 2016. Insertional Mutagenesis Identifies a STAT3/Arid1b/β-catenin Pathway Driving Neurofibroma Initiation. Cell Rep. 14, 1979-1990.
Yacoub, A., et al., 2019. Pegylated interferon alfa-2a for polycythemia vera or essential thrombocythemia resistant or intolerant to hydroxyurea. Blood 134, 1498-1509.
Zafra, M. P., et al., 2018. Optimized base editors enable efficient editing in cells, organoids and mice. Nat. Biotechnol. 36, 888-893.
Zhang, W. C., Slack, F. J., 2016. ADARs Edit MicroRNAs to Promote Leukemic Stem Cell Activity. Cell Stem Cell 19, 141-142.
Zhang, H., Meltzer, P., Davis, S., 2013. RCircos: an R package for Circos 2D track plots. BMC Bioinformatics 14, 244.
Zhao, H., et al, 2014. CrossMap: a versatile tool for coordinate conversion between genome assemblies. Bioinformatics 30, 1006-1007.
Zhou, C., et al., 2019. Off-target RNA mutation induced by DNA base editing and its elimination by mutagenesis. Nature 571, 275-278.
Zipeto, M. A., et al., 2016. ADAR1 Activation Drives Leukemia Stem Cell Self-Renewal by Impairing Let-7 Biogenesis. Cell Stem Cell 19, 177-191

A number of embodiments of the invention have been described. Nevertheless, it can be understood that various modifications may be made without departing from the spirit and scope of the invention. Accordingly, other embodiments are within the scope of the following claims.

1. A method for:
treating and ameliorating a cancer,
in vivo inhibition of myeloproliferative neoplasm (MPN) or AML stem cell propagation; or
the in vivo inhibition pre-leukemia stem cell (pre-LSC) transformation into leukemia stem cells (LSCs),
comprising:
administration to an individual in need thereof a formulation, a pharmaceutical composition or therapeutic combination of drugs, comprising:
(a) (i) imetelstat or imetelstat sodium, or imetelstat or imetelstat sodium and (ii) at least one second drug;
(b) (i) a JAK2 (Janus kinase 2) inhibitor, and (ii) at least one second drug,
(c) (i) fedratinib or fedratinib and (ii) at least one second drug,
(d) (i) 8-aza-adenosine, or 8-aza-adenosine and (ii) at least one second drug,
(e) (i) an integrase inhibitor, or raltegravir, or raltegravir and (ii) at least one second drug,
(f)(i) an integrase inhibitor, or dolutegravir, or dolutegravir and (ii) at least one second drug,
(g) an ADAR1 (adenosine deaminase acting on RNA-1) inhibiting agent,
(h) a compound having the formula, 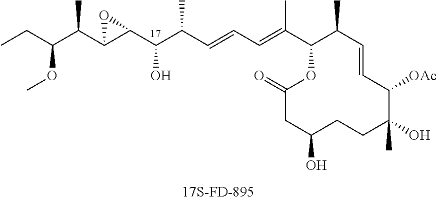 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]
or an enantiomer, deuterated version, stereoisomer, or salt thereof, or
(i) any combination thereof, or the therapeutic combination of drugs comprise (a) and (b), (a) and (c), (a) and (d), (a) and (e), (a) and (f), (a) and (g), (a) and (h), (b) and (c), (b) and (d), (b) and (e), (b) and (f), (b) and (g), (b) and (h), (c) and (d), (c) and (e), (c) and (f) and (c) and (g), (c) and (h), (d) and (e), (d) and (f), (d) and (g), (d) and (h), (e) and (f), (e) and (g), (e) and (h), (f) and (g), (f) and (h), and/or (g) and (h).

2. The method of claim 1, wherein the at least one second drug comprises an ATP-competitive protein tyrosine kinase inhibitor.

3. The method of claim 1, wherein the at least one second drug comprises a chemotherapeutic agent.

4. The method of claim 1, wherein the at least one second drug comprises a hypomethylating agent (HMA), wherein optionally the HMA comprises azacitidine or decitabine.

5. The method of claim 1, wherein the at least one second drug comprises a second telomerase inhibitor, wherein optionally the telomerase inhibitor comprises at least one, two or three of: zidovudine, stavudine, tenofovir, didanosine, abacavir, TMPI, telomestatin, RHPS4, BRACO-19, TMPyP4, tertomotide, ASTVAC-1, GX-301, UCPVax, UV-1, Vx-001, Vx-006, INO-1400, INVAC-1, ASTVAC-2, Telin(ab 4,4-dichloro-1-(2,4-dichlorophenyl)-3-methyl-5-pyrazolone), Vbx-011, Vbx-021, Vbx-026INO-5401, KML-001, TK-005, Vbx-016, ZI-HX, ZI-H04, and ZIH-03.

6. The method of claim 1, wherein the formulation, pharmaceutical composition or therapeutic combination of drugs or an active agent or drug contained therein is or are formulated or contained in: a liquid formulation (optionally sterile saline or water), a spray, a powder, an aerosol, a mist, or any formulation for inhalation, a pill, a capsule, a tablet, or a geltab, or equivalents; or, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; or, a drug delivery device or package, blister pack, clamshell or tray comprising a plurality of compartments spatially arranged on the drug delivery device or package, blister pack, clamshell or tray to follow a dosage administration regimen.

7. The method of claim 1, wherein an active agent or drug in the formulation, pharmaceutical composition or therapeutic combination of drugs is dosages at between about 10 to 500 mg/day, or between about 500 to 1 gram a day, or at a dosage of between about 100 to 600 mg per day or per dosage, or at about 100, 200, 300, 400, 500 or 600 mg per day or per dosage, and optionally a unit dosage is administered to an individual in need thereof once a day (QD), or twice a day (BID), or three times a day (TID), or more.

8. The method of claim 1, wherein an active agent or drug in the formulation, pharmaceutical composition or therapeutic combination of drugs is administered as or formulated with or formulated as an inhaled or aerosol formulation such as a powder or a mist or aerosol, and/or is formulated with or formulated as an oral, intramuscular (IM), subcutaneous (SC), intrathecal or intravenous (IV) formulation, wherein optionally both the inhaled (or aerosol) and the oral, IV, SC, intrathecal and/or IM formulations are administered simultaneously or sequentially.

9-10. (canceled)

11. A method for inhibiting replication of a virus, or for treating or ameliorating, or lessoning the symptoms of, or slowing the progress of, a viral infection in an individual in need thereof, wherein optionally the virus is flu (influenza) virus, a DNA or an RNA virus, a coronavirus, optionally a SARs-CoV-2 or COVID-19 virus or variant thereof, or a retrovirus, comprising:
administering to the individual in need thereof:
a vector or a recombinant virus, optionally a recombinant lentivirus or adenovirus or adeno-associated virus (AAV) vector, expressing or overexpressing (or capable of expressing or overexpressing) ADAR1 or an ADAR1 catalytic domain, wherein optionally the vector or recombinant virus are administered intravenously (IV), or
a transduced stem cell comprising cord blood CD34+ cells or mesenchymal stromal cells, wherein the cord blood CD34+ cells or mesenchymal stromal cells have contained therein a vector or a recombinant virus, optionally a recombinant lentivirus, expressing or overexpressing (or capable of expressing or overexpressing) ADAR1, wherein optionally the cord blood CD34+ cells or mesenchymal stromal cells are administered intravenously (IV).

12. A method for inhibiting replication of a virus, or for treating or ameliorating, or lessoning the symptoms of, or slowing the progress of, a viral infection in an individual in need thereof, wherein optionally the virus is flu (influenza) virus, a DNA or an RNA virus, a coronavirus, optionally a SARs-CoV-2 or COVID-19 virus or variant thereof, or a retrovirus, comprising:
administering to the individual in need thereof:
a ADAR1 full length protein, or an ADAR1 catalytic domain, or a ADAR1 Z alpha domain deleted-protein, contained in a liposome or equivalent lipid vesicle by intravenous administration or inhalation.

13. A method of claim 1, wherein the vector or recombinant virus, or liposome or equivalent lipid vesicle, or formulation, pharmaceutical composition or therapeutic combination of drugs, is or are administered to an individual in need thereof:
using a drug delivery device, optionally by inhalation, wherein the drug delivery device optionally comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device, or
intravenously (IV) or intramuscularly (IM).

14-15. (canceled)

16. The method of claim 1, wherein the ADAR1 inhibiting (inhibitory) nucleic acid is contained in and expressed by a vector.

17. The method of claim 16, wherein the lentiviral vector, is a lentiviral shRNA ADAR1 knockdown vector, a lentiviral ADAR1 inhibitory mutant vector, a lentiviral ADAR1 Z alpha domain deleted vector, or a lentiviral JAK2 overexpression vector.

18. The method of claim 17, wherein the ADAR1 inhibiting comprises an interferon inhibitory compound.

19. The method of claim 1, wherein the cancer is a leukemia, optionally acute myeloid leukemia (AML) or a myeloproliferative neoplasm (MPN).

20. The method of claim 1, wherein the second drug comprises an ATP-competitive protein tyrosine kinase inhibitor.

21. The method of claim 1, wherein the JAK2 (Janus kinase 2) inhibitor comprises ruxolitinib.

22. The method of claim 1, wherein the ADAR1 inhibiting agent comprises an ADAR1 inhibiting nucleic acid, and optionally the ADAR1 inhibiting agent comprises an antisense ADAR1 or a small inhibitory ADAR1 RNA.

23. The method of claim 2, wherein the ATP-competitive protein tyrosine kinase inhibitor comprises dasatinib.

24. The method of claim 3, wherein the chemotherapeutic agent comprises one, two, three or more of: afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, fedratinib, floxuridine, fludarabine, fluorouracil, gefitinib, gemcitabine, hydroxyurea, ibrutinib, icotinib, idarubicin, ifosfamide, imatinib, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib, vincristine, vinblastine, vinorelbine, and/or vindesine
